SEQUENCE LISTING (30 sequences)

SEQ ID NO: 1 - protein, 169 aa, Eimeria tenella
MGEADTQAWDTSVREWLVDTGRVFAGGVASIADGCRLFGAAVEGEGNAWEELVKTNYQIEVPQEDGTSISVDCDEAETLRQAVVDGRAPNGVYIGGTKYKLAEVKRDFTFNDQNYDVAILGKNKGGGFLIKTPNENVVIALYDEEKEQNKADALTTALNFAEYLHQSGF

SEQ ID NO: 2 - protein, 170 aa, Eimeria acervulina
MGEEADTQAWDTSVKEWLVDTGKVYAGGIASIADGCRLFGAAIDNGEDAWSQLVKTGYQIEVLQEDGSSTQEDCDEAETLRQAIVDGRAPNGVYIGGIKYKLAEVKRDFTYNDQNYDVAILGKNKGGGFLIKTPNDNVVIALYDEEKEQNKADALTTALAFAEYLYQGGF

SEQ ID NO: 3 - protein, 20 aa, Bovine
EWLVDTGKVFAGGVASIADG

SEQ ID NO: 4 - protein, 31 aa, Bovine
RMFGASTDSGGDPNAELVKAGYQIESVQEDN

SEQ ID NO: 5 - protein, 4 aa, Bovine
QAIV

SEQ ID NO: 6 - protein, 11 aa, Bovine
APDGVYIGGVK

SEQ ID NO: 7 - protein, 42 aa, Bovine
GGGFLIKTPNENIAIALYDEEKEQNKADALTTALNFADFLYQ

SEQ ID NO: 8 - protein, 152 aa, Plasmodium falciparum 3D7
LLATNQVSGAGLASEEDGVVYACVAQGEESDPNFDKWSLFYKEDYDIEVEDENGTKTTKTINEGQTILVVFNEGYAPDGVWLGGTKYQFINIERDLEFEGYNFDVATCAKLKGGLHLVKVPGGNILVVLYDEEKEQDRGNSKIAALTFAKEL

SEQ ID NO: 9 - protein, 152 aa, plas; identical to SEQ ID NO: 8

SEQ ID NO: 10 - protein, 116 aa, plas
CVAQGEESDPNFDKWSLFYKEDYDIEVEDENGTKTTKTINEGQTILVVFNEGYAPDGVWLGGTKYQFINIERDLEFEGYNFDVATCAKLKGGLHLVKVPGGNILVVLYDEEKEQDR

SEQ ID NO: 11 - protein, 131 aa, Hevea brasiliensis
MSWQTYVDDHLMCDIDGHRLTAAAIIGHDGSVWAQSSSFPQFKSDEVAAVMKDFDEPGSLAPTGLHLGGTKYMVIQGEPGAVIRGKKGSGGITVKRTGQALIIGIYDEPLTPGQCNMIVERLGDYLLDQGL

SEQ ID NO: 12 - protein, 130 aa, Arabidopsis thaliana
  misc_feature (11)..(11): Xaa can be any naturally occurring amino acid
SWQSYVDDHLXCDVEGNHLTAAAILGQDGSVWAQSAKFPQLKPQEIDGIKKDFEEPGFLAPTGLFLGGEKYXVIQGEQGAVIRGKKGPGGVTIKKTNQALVFGFYDEPXTGGQCNLVVERLGDYLIESEL

SEQ ID NO: 13 - protein, 133 aa, Betula pendula
MSWQTYVDEHLMCDIDGQGEELAASAIVGHDGSVWAQSSSFPQFKPQEITGIMKDFEEPGHLAPTGLHLGGIKYMVIQGEAGAVIRGKKGSGGITIKKTGQALVFGIYEEPVTPGQCNMVVERLGDYLIDQGL

SEQ ID NO: 14 - protein, 131 aa, Arabidopsis thaliana
MSWQSYVDDHLMCDVEGNHLTAAAILGQDGSVWAQSAKFPQLKPQEIDGIKKDFEEPGFLAPTGLFLGGEKYMVIQGEQGAVIRGKKGPGGVTIKKTNQALVFGFYDEPMTGGQCNLVVERLGDYLIESEL

SEQ ID NO: 15 - DNA, 999 bp, Eimeria acervulina
tgtttctcttcattgtttgtagtttctttgtatttccttactcagttaaaatgggtgaagaggctgatactcaggcgtgggatacctcagtgaaggaatggctcgtggatacggggaaggtatacgccggcggcattgctagcattgcagatgggtgccgcctgtttggcgctgcaatagacaatggggaggatgcgtggagtcagttggtgaagacaggatatcagattgaagtgcttcaagaggacggctcttcaactcaagaggactgcgatgaagcggaaaccctgcggcaagcaattgttgacggccgtgccccaaacggtgtttatattggaggagttaaatataaactcgcagaagttaaacgtgatttcacctataacgaccagaactacgacgtggcgattttggggaagaacaagggtggcggtttcctgattaagactccgaacgacaatgtggtgattgctctttatgacgaggagaaggagcagaacaaagcagatgcgctgacaacggcacttgccttcgctgagtacctgtaccagggcggcttctaattgatctccagtgcacaaccacttgatgagaaggaaaaacctttcataacaacgacttccccccagtgttaccacacagggagaagagagacgcacaacttctctacaaatagcggacagcgtattgcacaccctgacctttgtttattgaagagggtgtagggggaggagcatcagcaggcagcagctttgggcggtctggacagttcgccatggagggagagctgtgtagacactcgagagcagcagcagcagcacggttaagtggcagacgcagagacgcctttgttgtacaacttctctctcacccgcgtttgttgtagagaggagtatttattatgaatgcatatccagcaaacaacgaggcaaacagcgggtgcttactgccgtgcaaatgatacgcacaccaccaaccatttaataagtgcttttctta

SEQ ID NO: 16 - DNA, 977 bp, Eimeria acervulina
cgtttctttgtatttccttactcagttaaaatgggtgaagaggctgatactcaggcgtgggatacctcagtgaaggaatggctcgtggatacggggaaggtatacgccggcggcattgctagcattgcagatgggtgccgcctgtttggcgctgcaatagacaatggggaggatgcgtggagtcagttggtgaagacaggatatcagattgaagtgcttcaagaggacggctcttcaactcaagaggactgcgatgaagcggaaaccctgcggcaagcaattgttgacggccgtgccccaaacggtgtttatattggaggaattaaatataaactcgcagaagttaaacgtgatttcacctataacgaccagaactacgacgtggcgattttggggaagaacaagggtggcggtttcctgattaagactccgaacgacaatgtggtgattgctctttatgacgaggagaaagagcagaacaaagcagatgcgctgacaacggcacttgccttcgctgagtacctgtaccagggcggcttctaattgatctccagtgcacaaccacttgatgagaaggaaaaacctttcataacaacaacttcccccagtgttgccacacagggagaagagagacgcacaacttctctacaaatagcggacagcgtattgcacaccctgacctttgtttattgaagagggtgtagggggaggagcatcagcaggcagcagctttgggcggtctggacagttcgccatggagggagagctgtgtagacactcgagagcagcagcagcagcacggttaagtggcagacgcagagacgcctttgttgtacaacttctctctcacccgcgtttgttgtagagaggagtatttattatgaatgcatatccagcaaacaacgaggcaaacagcgggtgcttactgccgtgcaaatgatacgcacaccaccaaccatttaataagtgcttttctt

SEQ ID NO: 17 - DNA, 1074 bp, Eimeria acervulina
ggcacgagtcttcattgtttgtagtttctttgtatttccttactcagttaaaatgggtgaagaggctgatactcaggcgtgggatacctcagtgaaggaatggctcgtggatacggggaaggtatacgccggcggcattgctagcattgcagatgggtgccgcctgtttggcgctgcaatagacaatggggaggatgcgtggagtcagttggtgaagacaggatatcagattgaagtgcttcaagaggacggctcttcaactcaagaggactgcgatgaagcggaaaccctgcggcaagcaattgttgacggccgtgccccaaacggtgtttatattggaggaattaaatataaactcgcagaagttaaacgtgatttcacctataacgaccagaactacgacgtggcgattttggggaagaacaagggtggcggtttcctgattaagactccgaacgacaatgtggtgattgctctttatgacgaggagaaagagcagaacaaagcagatgcgctgacaacggcacttgccttcgctgagtacctgtaccagggcggcttctaattgatctccagtgcacaaccacttgatgagaaggaaaaacctttcataacaacaacttcccccagtgttgccacacagggagaagagagacgcacaacttctctacaaatagcggacagcgtattgcacaccctgacctttgtttattgaagagggtgtagggggaggagcatcagcaggcagcagctttgggcggtctggacagttcgccatggagggagagctgtgtagacactcgagagcagcagcagcagcacggttaagtggcagacgcagagacgcctttgttgtacaacttctctctcacccgcgtttgttgtagagaggagtatttattatgaatgcatatccagcaaacaacgaggcaaacagcgggtgcttactgccgtgcaaatgatacgcacaccaccaaccatttaataagtgcttttcttaatatggcttgacgctcccagcgaaaaaaaaaaaaaaaaaaaaaaaaaaaaaaaaaaaaaaaaaaaaaaaaaaaa

SEQ ID NO: 18 - DNA, 460 bp, Eimeria acervulina
  misc_feature (240)..(241): n is a, c, g, or t
tgttgacggccgtgccccaaacggtgtttatattggaggagttaaatataaactcgcagaagttgtaagtttccttcatactctagaagaatagcgcttgctcatccatggtgtcgtgcagtgggatgcaatcgccacgcggggctgtacagacacctcaaagttgaatggtagtaataatagtcatgttcttcatgatgatggaataagtgaataattagggtgttttgtgacggcgtnntcgcttttttgtcattttcgtcgtttctcttttgtttatttcgggccgatgatgcagaaacgtgatttcacctataacgaccagaactacgacgtggcgattttggggaagaacaagggtggcggtttcctgattaagactccgaacgacaatgtggtgattgctctttatgacgaggagaaggagcagaacaaagcagatgcgctgacaacgcacccc

SEQ ID NO: 19 - DNA, 529 bp, Eimeria tenella
  misc_feature (406)..(406): n is a, c, g, or t
ttattttctatttagtttgcaaaatgggagaagcagacacccagcctgggacacttcggtccgcgagtggctggttgacaccggcagggtcttcgccggcggcgttgctagcatagccgacggctgccggctcttcggagcagcagtggagggcgagggcaacgcctgggaagaactcgtcaagaccaactaccaaattgaagtcccccaggaagacggaacctccatttcagtggattgcgacgaggccgagactctgcggcaggcggtggtggacggccgcgcgcccaacggcgtctacatcggcggcaccaagtacaagctcgccgaagtcaaaagggacttcaccttcaacgaccaaaactatgatgtggcgattctgggaaaaaacaaaggcggagggttnttgattaaaactccaaacgaaaatgttgttatagctttgtatgatgaagaaaaagaacataacaaagctgatgctctcacaacagctcttaacttcgcggagtatctgtaccaaggaagcttc

SEQ ID NO: 20 - DNA, 558 bp, Eimeria tenella
tccagtttttgctttcttttccaaattattttctatttagtttgcaaaatgggagaagcagacacccaggcctgggacacttcggtccgcgagtggctggttgacaccggcagggtcttcgccggcggcgttgctagcatagccgacggctgccggctcttcggagcagcagtggagggcgagggcaacgcctgggaagaactcgtcaagaccaactaccaaattgaagtcccccaggaagacggaacctctatttcagtggattgcgacgaggcggagactctgcggcaggcggtggtggacggccgcgcgcccaacggcgtctacatcggcggcaccaagtacaagctcgccgaagtcaaaagggacttcaccttcaacgaccaaaactatgatgtggcgattctgggaaaaaacaaaggcggagggtttttgattaaaactccaaacgaaaatgttgttatagctttgtatgatgaagaaaaagaacaaaacaaagctgatgctctcacaacagctcttaacttcgcggagtaccttcaccagtccggcttctaa

SEQ ID NO: 21 - DNA, 529 bp, Eimeria tenella; identical to SEQ ID NO: 19
  misc_feature (406)..(406): n is a, c, g, or t

SEQ ID NO: 22 - DNA, 529 bp, Toxoplasma gondii
  misc_feature (483)..(483): n is a, c, g, or t
ttttcgcttctttcgcggcttgtttccattcttttccaagatgtccgactgggaccctgttgtcaaggagtggcttgttgacacaggctactgctgcgcaggcggcatcgccaacgcggaggacggtgttgtgttcgccgcggcggctgatgatgatgacggatggtccaagctgtacaaggatgatcatgaggaggacactatcggagaggatggcaacgcgtgcggcaaggtgtcgatcaacgaggcctccacgatcaaagctgcagttgacgatggcagtgcccctaacggtgtttggattggcggccagaagtacaaggttgtccgacctgagaaaggattcgagtacaacgactgcaccttcgacatcaccatgtgtgcacggtccaagggtggcgcgcacttgatcaagaccccgaatggctctatcgtcattgccctttacgatgaggagaaggaacaggacaagggaaacagcangacttcggcattggcctttgccgagtatcttcaccagtctgggtac

SEQ ID NO: 23 - DNA, 509 bp, Toxoplasma gondii
tgtttccattcttttccaagatgtccgactgggaccctgttgtcaaggagtggcttgttgacacaggctactgctgcgcaggcggcatcgccaacgcggaggacggtgttgtgttcgccgcggcggctgatgatgatgacggatggtccaagctgtacaaggatgatcatgaggaggacactatcggagaggatggcaacgcgtgcggcaaggtgtcgatcaacgaggcctccacgatcaaagctgcagttgacgatggcagtgcccctaacggtgtttggattggcggccagaagtacaaggttgtccgacctgagaaaggattcgagtacaacgactgcaccttcgacatcaccatgtgtgcacggtccaagggtggcgcgcacttgatcaagaccccgaatggctctatcgtcattgccctttacgatgaggagaaggaacaggacaagggaaacagcaggacttcggcattggcctttgccgagtatcttcaccagtctgggtac

SEQ ID NO: 24 - DNA, 549 bp, Cryptosporidium parvum
ctcaatcagaacttatattattttatcttttactccgctatttttatattttacttaattaattaaagatgtctgaatgggatgatatggtcaaagaatggttaattgacaccggtagtgtatgtgctggtggtctttgttcaatagatggtgcattctatgctgcttctgctgatcaaggtgatgcctggaagactcttgttagagaagatcatgaagagaatgttattcaatctgacggagtttcagaggctgctgaattaattaatgatcaaactacactatgccaagcaatctccgaaggtaaggcaccaaacggcgtttgggtcggaggaaacaaatataagattatccgtgtagagaaggacttccagcaaaacgatgctaccgttcatgttacattttgtaacagacctcaaggtggatgttttttagttgatactcaaaacggtactgttgttgttgcagtttacgacgaatccaaggatcaatcatcaggtaattgcaagaagttgctctgcaactagccgagtacctcgtatctcaggg

SEQ ID NO: 25 - DNA, 487 bp, Plasmodium falciparum
ggcagaagaatattcatgggacagttatttaaatgatcgccttttagcaaccaatcaagtttcaggagctggattagcttcggaagaagatggagttgtctatgcttgtgtagctcagggtgaagagagtgacccaaattttgataaatggtcacttttttataaagaagattatgatattgaagttgaagatgaaaatggtactaaaactaccaaaacgataaatgaaggacaaacgatcctggtcgtttttaatgaaggatatgctcctgatggagtttggttaggtggtactaaatatcaatttataaatattgaaagagatttagaatttgaaggttataattttgatgtagctacttgtgctaaattaaaaggtggtcttcacttggtgaaagttccaggaggaaatatattagttgtattatatgatgaagaaaaagagcaagacagaggaaattccaaaatcgctgccttgacttttgca

SEQ ID NO: 26 - protein, 114 aa, Artificial Sequence
  Artificial protein for antibody generation
EWLVDTGKVFAGGVASIADGRMFGASTDSGBDPNAELVQYNAGYQIESVQEDNGTVQQAIVAPDGVYIGGVKGGGFLIKTPNENIAIALYDEEKEQNKADALTTALNFADFLYQ

SEQ ID NO: 27 - DNA, 513 bp, Eimeria tenella
atgggtgaagaggctgatactcaggcctgggacacttcggtccgcgagtggctggttgacaccggcagggtcttcgccggcggcgttgctagcatagccgacggctgccggctcttcggagcagcagtggagggcgagggcaacgcctgggaagaactcgtcaagaccaactaccaaattgaagtcccccaggaagacggaacctctatttcagtggattgcgacgaggcggagactctgcggcaggcggtggtggacggccgcgcgcccaacggcgtctacatcggcggcaccaagtacaagctcgccgaagtcaaaagggacttcaccttcaacgaccaaaactatgatgtggcgattctgggaaaaaacaaaggcggagggtttttgattaaaactccaaacgaaaatgttgttatagctttgtatgatgaagaaaaagaacaaaacaaagctgatgctctcacaacagctcttaacttcgctgagtacctgtaccagggcggcttctaa

SEQ ID NO: 28 - protein, 169 aa, Eimeria tenella
MGEEADTQAWDTSVREWLVDTGRVFAGGVASIADGCRLFGAAVEGEGNAWEELVKTNYQIEVPQEDGTSISVDCDEAETLRAVVDGRAPNGVYIGGTKYKLAEVKRDFTFNDQNYDVAILGKNKGGGFLIKTPNENVVIALYDEEKEQNKADALTTALNFAEYLYQGGF

SEQ ID NO: 29 - protein, 19 aa, Bovine
TPNENIAIALYDEEKEQNK

SEQ ID NO: 30 - protein, 168 aa, Eimeria tenella
GEEADTQAWDTSVREWLVDTGRVFAGGVASIADGCRLFGAAVEGEGNAWEELVKTNYQIEVPQEDGTSISVDCDEAETLRAVVDGRAPNGVYIGGTKYKLAEVKRDFTFNDQNYDVAILGKNKGGGFLIKTPNENVVIALYDEEKEQNKADALTTALNFAEYLYQGGF

(19) United States
(12) Patent Application Publication
(10) Pub. No.: US 2005/0058658 A1
(43) Pub. Date: Mar. 17, 2005

(54) Compositions and methods for immunotherapy of human immunodeficiency virus (HIV)

(75) Inventor: Barnett Rosenberg, Holt, MI (US)

Correspondence Address: DARBY & DARBY P.C., P. O. BOX 5257, NEW YORK, NY, 10150-5257, US

(73) Assignee: Barros Research Institute, Holt, MI (US)

(21) Appl. No.: 10/893,581

(22) Filed: Jul. 15, 2004

Related U.S. Application Data
(60) Provisional application No. 60/487,865, filed on Jul. 15, 2003.

(51) Int. Cl. A61K 39/21

(52) U.S. Cl. 424/188.1

(57) ABSTRACT

The present invention provides compositions and methods for the prevention and treatment of an infectious disease caused by infection with HIV and for stimulating an immune response in a subject. In particular, the present invention provides Apicomlexa-related proteins (ARPs) that have immune stimulatory activity and thus have uses in the treatment and prevention of an infectious disease caused by infection with HIV and in immune modulation. Compositions comprising an ARP are provided. Methods of use of an ARP for the prevention and/or treatment of an infectious disease caused with infection with HIV, and for eliciting an immune response in a subject, are also provided.

This application claims the priority of U.S. application Ser. No. 60/487,865 filed on Jul. 15, 2003.

### 1. FIELD OF THE INVENTION

The present invention relates to compositions and methods for stimulating an immune response and the prevention and treatment of an infectious disease caused by infection with Human Immunodeficiency Virus (HIV). More particularly, the present invention relates to compositions comprising Apicomplexa-related proteins (ARPs), a fragment, a derivative, a homolog or an analog thereof, and their uses in immune stimulation, and the prevention and treatment of HIV infection.

### 2. BACKGROUND OF THE INVENTION

### 2.1 HIV

Human immunodeficiency virus (HIV) has been identified as the etiological agent responsible for acquired immune deficiency syndrome (AIDS), a fatal disease characterized by destruction of the immune system and the inability to fight off life-threatening, opportunistic infections. According to estimates from the Joint United Nations Program on HIV/AIDS (UNAIDS) and the World Health Organization (WHO), 38.6 million adults and 3.2 million children were living with HIV at the end of 2002. In addition to the large number of individuals already infected, the virus continues to spread. Africa is one of the hardest hit regions in the world with this epidemic. AIDS is now the leading cause of mortality in the sub-Saharan region. By the end of the year 2001, the total number of people living in this region with HIV/AIDS was reported to be 28.5 million.

HIV is a member of the lentiviruses, a subfamily of retroviruses. It integrates its genetic information into the genome of the host. Most importantly, it infects and invades cells of the immune system, resulting in break-down of the body's immune system and rendering the patient susceptible to opportunistic infections and neoplasms. HIV-1 is cytopathic for T4 lymphocytes, cells of the immune system that express the cell surface differentiation antigen CD4. In addition to CD4+ T cells, the host range of HIV includes cells of the mononuclear phagocytic lineage, including blood monocytes, tissue macrophages, Langerhans cells of the skin, and dendritic reticulum cells within lymph nodes.

Researchers have engaged in studies to develop clinically effective antiviral agents and vaccines. Examples of medicaments currently under clinical use or clinical trail are non-nucleotide analog inhibitors of reverse transcriptase, such as Nevirapine (BI-RG-587), TIBO (R82913), pyrinodes (such as R-697,661 and L-696,227), bis(heteroary) piperazines (BHAPs, such as U-87201E and U-90,152), atevirdine mesylate (ATV) and R-89431; HIV protease inhibitors, include substrate analogs and non-analogs, such as Ro 31-8959, A-77003 and A-80987; HIV Tat protein inhibitors, such as Ro 5-3335 and Ro 27-7429; blockers of viral entry into cells, such as soluble CD4 protein (sCD4), and chimeric sCD4 derivatives, such as CD4-IgG and CD4-PE40; blockers of HIV RNaseH activity, such as the AZT derivative azidothymidine monophosphate; drugs that alter the intracellular milieu to create conditions less favorable for viral replication, such as the free-radical scavengers and glutathione-level restoring drugs (N-acetylcysteine and similar drugs), and thalidomine (which seems to lower blood TNF-α levels). Efforts have also focused on manipulation of the immune system and viral replication with naturally-occurring cytokines and lymphokines, or other agonists or antagonists of these systems. One of the drugs most frequently used is azidothimidine (AZT).

AZT was approved as an anti-HIV agent because its administration significantly lowers mortality. However, the virus develops resistance to AZT, and the drug has significant, unpleasant side effects. Antiviral therapies with pharmaceuticals that inhibit the replication of HIV have been found to decrease plasma virus to undetectable levels. Combination antiviral HIV therapy with Protease Inhibitors (PI) and Reverse Transcriptase (RT) inhibitors has provided Highly Active Antiviral Therapy (HAART) (Hammer, S. M. et al., N. Engl. J. Med. 1997, 337:725-733; Gulick, R. M. et al., N. Engl. J. Med. 1997, 337:734-739). HAART results in the rapid cessation of viral replication and the decline of plasma virus to undetectable levels within 4-8 weeks. However, in most instances, HAART alone does not lead to complete immune recovery. As effective as this approach has proven, HIV will develop resistance to it. Moreover, it now appears that many individuals may not be able to take HAART indefinitely, due to serious long-term side effects. As many as 40%-60% of patients who have received HAART for greater than one year have developed symptoms of Cushing's Syndrome, with hyperglycemia, hyperlipidemia, centipetal fat distribution, and peripheral muscle wasting (P. Yeni, Fourth International AIDS Congress, 1998). HAART for an indefinite period of time is no longer feasible.

Accordingly, there is a need for new safe and efficient therapeutic and preventive methods for HIV-infections. The invention addresses this need in the field.

### 2.2 Protozoa and Immune Responses of their Hosts

Parasites have evolved multiple evasion strategies allowing them to survive in their primary host and to complete their life cycle through a series of stages that usually involve passage through the environment and often through secondary hosts. These strategies include antigen shedding, antigen shifting, living intracellularly for long periods, manipulating host cell biochemistry, infecting multiple organs, and encystment (see, e.g., Beverley, Cell, 1996 87:787-789; Zambrano-Villa et al., Trends in Parasitol. 2002, 18:272-278). Such mechanisms have been fine-tuned through evolutionary pressures to help the protozoan avoid the immune response of the host. In many cases, protozoans are so specialized in these avoidance mechanisms that they can only complete their life cycle in one host species or genera.

Apicomplexa are spore-forming single-celled parasites of animals and include species that cause various diseases, such as malaria, coccidiosis, redwater disease, corridor disease, east coast fever and binary fever. Four major classes within the phylum Apicomplexa are: Coccidia (genera include, e.g., Besnoitia, Caryospora, Cryptosporidium, Eimeria, Frenkelia, Hammondia, Hepatozoon, Isospora, Lankesterella, Neospora, Sarcocystis, and Toxoplasma); Gregarinia (genera include, e.g., Gregarina, Monocystis, Ophriocystis, and Pseudomonocystis); Haemosporida or haemosporidians (genera include, e.g., Haemoproteus, Hepatocystis, and Plasmodium); and Piroplasmida or Piroplasmids (genera include, e.g., Babesia, Cytauxzoon, and Theileria). Seven species that infect humans have been identified (Plasmodium, Babesia, Cryptosporidium, Isospora, Cyclospora, Sarcocystis, Toxoplasma).

A defining characteristic of the Apicomplexa is a group of organelles found at one end—called the apical end—of the organism. This “apical complex” includes secretory organelles known as micronemes and rhoptries, polar rings composed of microtubules, and in some species a conoid which lies within the polar rings. At some point during their life cycle, members of the Apicomplexa either invade or attach to host cells. The apical organelles play a role in these host-parasite interactions.

A few Eimeria species are known to cause severe morbidity and occasionally mortality in certain animals. In particular, E. tenella, E. acervulina, and E. maxima are a problem in the chicken industry. The stress and crowded conditions exacerbate Eimeria infections and cause massive diarrhea and death. A similar problem can occur in the cattle industry, where crowding among calves can lead to severe disease. Usually, adult animals are immune to Eimeria but a low, asymptomatic infection is endemic in most vertebrates. Upon first exposure, a brief humoral immune response has been documented in animals (Faber et al., Vet. Parasitol. 2002, 104:1-17; Allen & Fetterer, Clin. Microbiol. Rev. 2002, 15:58-65; Rose, Eimeria, Isopora, and Cryptosporidium, in: Immune Responses in Parasitic Infections: Immunology, Immunopathology, and Immuneoprophylaxis (ed. E. J. L. Soulsby) 1987, pp. 275-230?), but this is replaced by a localized cellular immune response that appears to provide lasting protection throughout a host's life. The exact mechanism of this protection is not fully known and why low levels of the organism survive without undergoing the explosive growth of its life cycle remains a mystery.

Aliberti et al. (2003, Nature Immunology 4(5): 485-490) showed a purified protein, C-18, which is an isoform of Toxoplasma gondii cyclophilin (which does not contain a profilin domain), can stimulate production of interleukin-12 (IL-12) from dendritic cells (DCs). Hoewever, recombinant T gondii C-18 showed reduced IL-12 stimulatory activity relative to the crude T. gondii tachyzoite extract (STAg).

### 2.3 The 19 kD Sporozoite Antigen

Vaccination against Eimeria has been the subject of substantial research in the animal husbandry of poultry, and to a lesser extent, cattle. Chemical prophylaxes are standard treatment in the poultry industry, but resistant strains of Eimeria are beginning to be a problem. The development of vaccines is considered an alternative approach and has focused on the use of surface antigens from the sporozoite stage, which is the stage that invades the epithelial layer of the small intestine (see e.g., Allen & Fetterer, Clin. Microbiol. Rev. 2002, 15:58-65; Blackman & Bannister, Mol. Biochem. Parasitol. 2001, 117:11-25; Min et al., Vaccine 2001, 20:267-274). Merozoite surface antigens are also of interest because this protozoan stage is released after the first round of multiplication and infects neighboring cells. During this process an effective immune response could limit the expansion of the infection.

One of the surface molecules, eventually examined for use as a vaccine, was isolated several times from cDNA expression libraries of E. acervulina. It was originally identified by Jenkins et al. (Exp. Parasitol. 1998, 66:96-107) as a low-abundance surface protein (clone cSZ-1) detected by rabbit antibodies made to acervulina membranes. Sera from infected chickens, however did not react with it. They identified two bands in Western blots at 240 and 160 kD and showed that the β-galactosidase fusion protein, made in E. coli, could activate T-cell division. Another group (Laurent et al., Mol. Bioch. Parasitol. 1994, 63:79-86) subsequently reported that a 19 kD surface protein, referred to as 1 P, was conserved across three poultry Eimeria species, shared the same cDNA sequence as the cSZ-1 clone of Jenkins et al., and suggested that iodination may have given the anomalously high molecular weights in the previous work. The nucleotide sequence of their clone was submitted to GenBank under the name of ‘19 kD sporozoite antigen’ (Accession Z26584). Most recently, Lillehoj et al. (Avian Diseases 2000, 44:379-389) isolated a slightly longer protein reading frame from a cDNA clone (3-1 E) that reacted with rabbit antiserum to E. acervulina merozoites (Accession Z26584). The translation included 17 additional amino acids at the N terminus, but the remainder of the molecule was virtually identical. They demonstrated that the 3-1 E protein activated cell mediated immunity by showing in vitro IFNγ production from spleen cell isolated from chickens doubly challenged with E. acervulina and then exposed to the protein. They also showed that the protein can be used as a modestly effective vaccine to prevent coccidiosis from Eimeria. This group continues to explore this potential using the nucleotide sequence as a DNA vaccine (Song, et al., Vaccine 2001, 19:234-252; Min, et al., Vaccine 2001, 20:267-274). As the sole agent, the DNA vaccine generated only partial protection from challenge. Addition of plasmids carrying various chicken cytokine genes offered some improvement, but still did not reduce oocyst production by more than two-fold.

Citation or discussion of a reference herein shall not be construed as an admission that such is prior art to the present invention.

### 3. SUMMARY OF THE INVENTION

The present invention provides a method of treating or preventing an infectious disease in a subject where the infectious disease is caused by infection with a human immunodeficiency virus (HIV). The method comprises administering to a subject in whom such treatment is desired a therapeutically or prophylactically effective amount of an ARP protein, e.g., a substance selected from the group consisting of (a) an isolated protein comprising SEQ ID NO:1 (ARP of E. tenella); (b) an isolated protein comprising SEQ ID NO:2 (ARP of E. acervulina); (c) an isolated protein comprising SEQ ID NO: 3, 4, 5, 6 and 7 (partial amino acid sequence of bovine Eimeria spp.). ARP SEQ ID NOs: 3-7 are not necessarily contiguous; there may be intervening or adjacent sequences to each fragment though in a specific embodiment, a protein comprises SEQ ID NOs:3, 4, 5, 6 and 7 in an order of SEQ ID NO:3 to SEQ ID NO:7 from the N terminus to the C terminus. In another embodiment, the substance is an isolated protein that is a product of a process comprising the steps of: (1) producing a cell extract of Eimeria infected tissue or cells; (2) centrifuging the cell extract to produce a supernatant; (3) partitioning the supernatant by ammonium sulfate to precipitate proteins to a pellet, and resuspending the pellet into a solution; (4) applying the solution to a hydrophobic interaction column (hereinafter “HIC column”); (5) eluting material bound to the HIC column to produce eluted material; (6) dialyzing the eluted material to produce a sample for loading on a diethylaminoethyl (hereinafter “DEAE”) column; (7) applying the sample to a DEAE column; (8) eluting material bound to the DEAE column with buffer to produce an eluted fraction and concentrating the eluted fraction; (9) applying the concentrated eluted fraction to a size exclusion column; (10) collecting and pooling fractions eluted from the size exclusion column; (11) applying the pooled fractions to a HIC column to remove calcium-binding proteins; (12) applying eluted fractions from the HIC column to a high performance liquid chromatography (hereinafter “HPLC”) HIC column and collecting active (immunostimulatory) fractions, wherein said active fractions have the ability to stimulate interleukin-12 release from dentritic cells, said dentritic cells having been isolated from a mammal within 10 hours of assaying said ability (or as assayed by one or more assays described in Section 5.6 and Section 13, infra); (13) dialyzing the active fractions; and (14) applying the dialyzed active fractions to a HPLC DEAE column and collecting one or more active (immunostimulatory) fractions, wherein said active fractions have the ability to stimulate interleukin-12 release from dentritic cells, said dentritic cells having been isolated from a mammal within 10 hours of assaying said ability (or as assayed by one or more assays described in Section 5.6 and Section 13, infra). In yet another embodment, the substance is an isolated protein that is the product of a process comprising the steps of: (1) producing homogenate of a purified Apicomplexan organism or a cell infected with an Apicomplexan organism; (2) centrifuging the homogenate to obtain a pellet; (3) treating the pellet with a solution comprising a detergent, a phospholipase C, or a protease; (4) centrifuging the solution to obtain a supernatant; (5) applying the supernatant to a reverse phase HPLC column; (6) eluting bound protein from the reverse phase HPLC column, to produce eluted protein, wherein said protein has the ability (immunostimulatory ability) to stimulate interleukin-12 release from dendritic cells, said dendritic cells having been isolated from a mammal within 10 hours of assaying said ability (or as assayed by one or more assays described in Section 5.6 and Section 13, infra). The invention also provides isolated nucleic acids comprising nucleotide sequences encoding any of the proteins set forth above, as well as cells transformed with such a nucleic acid, which nucleic acid has a nucleotide sequence which is preferably operably linked to a promoter. These nucleic acids and cells can be used in treating or preventing HIV infection.

The present invention also provides a method of treating or preventing an infectious disease in a subject where the infectious disease is caused by infection with a human deficiency virus (HIV), comprising administering to a subject in whom such treatment or prevention is desired a therapeutically or prophylactically effective amount of a substance selected from the group consisting of (a) an isolated protein comprising SEQ ID NO:3, 4, 5, 6 or 7; (b) an isolated protein comprising a variant of SEQ ID NO:1 or SEQ ID NO:2, wherein said variant has only conservative amino acid substitutions relative to SEQ ID NO:1 or SEQ ID NO:2, respectively; (c) an isolated protein comprising a variant of SEQ ID NO:3, 4, 6 or 7, wherein said variant has only conservative amino acid substitutes relative to SEQ ID NO:3, 4, 6, or 7, respectively; (d) an isolated protein that has at least 25%, at least 30%, at least 35%, at least 40%, at least 45%, at least 50%, at least 55%, at least 60%, at least 65%, at least 70%, at least 75%, or at least 80% identity to the sequence of SEQ ID NO:1 or SEQ ID NO:2, as determined by a BLAST 2.0 algorithm set to default parameters; (e) an isolated protein comprising a PROF (profiling domain); (f) an isolated Apicomplexan protein comprising an amino acid sequence encoded by a nucleic acid consisting of a nucleotide sequence hybridizable to SEQ ID NO:15, 16, 17, 18, 19, 20, or 27, or a complement of any of the foregoing SEQ ID NOs, under conditions of low stringency; (g) an isolated nucleic acid comprising a nucleotide sequence encoding any of (a)-(f) above; (h) a cell transformed with a nucleic acid comprising a nucleotide sequence encoding any of (a)-(f) above, said nucleotide sequence being operably linked to a promoter; and wherein said substance has immune stimulatory ability, e.g., has the ability to stimulate interleukin-12 release from dentritic cells, said dendritic cells having been isolated from a mammal witin 10 hours of assaying said ability.

The present invention also provides a method of stimulating an immune response comprising administering to a subject infected with a human immunodeficiency virus (HIV), an effective amount of a substance selected from the group consisting of: (a) an isolated protein comprising SEQ ID NO:1 (ARP of E. tenella); (b) an isolated protein comprising SEQ ID NO:2 (ARP of E. acervulina); (c) an isolated protein comprising SEQ ID NO: 3, 4, 5, 6 and 7 (partial amino acid sequence of bovine Eimeria spp. ARP); (d) an isolated protein that is a product of process comprising the steps of: (1) producing a cell extract of Eimeria infected tissue or cells; (2) centrifuging the cell extract to produce a supernatant; (3) partitioning the supernatant by ammonium sulfate to precipitate proteins to a pellet, and resuspending the pellet into a solution; (4) applying the solution to a HIC column; (5) eluting material bound to the HIC column to produce eluted material; (6) dialyzing the eluted material to produce a sample for loading on a DEAE column; (7) applying the sample to a DEAE column; (8) eluting material bound to the DEAE column with buffer to produce an eluted fraction and concentrating the eluted fraction; (9) applying the concentrated eluted fraction to a size exclusion column; (10) collecting and pooling fractions eluted from the size exclusion column; (11) applying the pooled fractions to a HIC column to remove calcium-binding proteins; (12) applying eluted fractions from the HIC column to a HPLC HIC column and collecting active (immunostimulatory) fractions, wherein said active fractions have the ability to stimulate interleukin-12 release from dendritic cells, said dendritic cells having been isolated from a mammal within 10 hours of assaying said ability (or as assayed by one or more assays described in Section 5.6 and Section 13, infra); (13) dialyzing the active fractions; and (14) applying the dialyzed active (immunostimulatory) fractions to a HPLC DEAE column and collecting one or more active fractions, wherein said active fractions have the ability to stimulate interleukin-12 release from dendritic cells, said dendritic cells having been isolated from a mammal within 10 hours of assaying said ability (or as assayed by one or more assays described in Section 5.6 and Section 12, infra). In yet another embodiment, the substance is an isolated protein that is the product of a process comprising the steps of: (1) producing a homogenate of a purified Apicomplexan organism or a cell infected with an Apicomplexan organism; (2) centrifuging the homogenate to obtain a pellet; (3) treating the pellet with a solution comprising a phospholipase; (4) centrifuging the solution to obtain a supernatant; (5) applying the supernatant to a reverse phase HPLC column; and (6) eluting bound protein from the reverse phase HPLC column, to produce eluted protein, wherein said protein has the ability (immunostimulatory ability) to stimulate interleukin-12 release from dendritic cells, said dendritic cells having been isolated from a mammal within 10 hours of assaying said ability (or as assayed by one or more assays described in Section 5.6 and Section 1, infra). The invention also provides isolated nucleic acids comprising a nucleotide sequence encoding any of the proteins set forth above as well as cells transformed with a nucleic acid, which nucleic acid has a nucleotide sequence which is preferably operably linked to a promoter. These nucleic acids and cells can be used to stimulate immune responses in subjects infected with HIV.

The present invention also provides a method of stimulating an immune response comprising administering to a subject infected with a human immunodeficiency virus (HIV), an effective amount of a substance selected from the group consisting of: (a) an isolated protein comprising SEQ ID NO: 3, 4, 5, 6 and 7; (b) an isolated protein that is a product of a process comprising the steps of: (1) producing a cell extract of Eimeria infected tissue or cells, wherein said Eimeria do not naturally infect an avian species; (2) centrifuging the cell extract to produce a supernatant; (3) partitioning the supernatant by ammonium sulfate to precipitate proteins to a pellet, and resuspending the pellet into a solution; (4) applying the solution to a HIC column; (5) eluting material bound to the HIC column to produce eluted material; (6) dialyzing the eluted material to produce a sample for loading on a DEAE column; (7) applying the sample to a DEAE column; (8) eluting material bound to the DEAE column with buffer to produce an eluted fraction and concentrating the eluted fraction; (9) applying the concentrated eluted fraction to a size exclusion column; (10) collecting and pooling fractions eluted from the size exclusion column; (11) applying the pooled fractions to a HIC column to remove calcium-binding proteins; (12) applying eluted fractions from the HIC column to a HPLC HIC column and collecting active (immunostimulatory) fractions, wherein said active fractions have the ability to stimulate interleukin-12 release from dendritic cells, said dendritic cells having been isolated from a mammal within 10 hours of assaying said ability (or as assayed by one or more assays described in Section 5.6 and Section 13, infra); (13) dialyzing the active fractions; and (14) applying the dialyzed active fractions to a HPLC DEAE column and collecting one or more active (immunostimulatory) fractions, wherein said active fractions have the ability to stimulate interleukin-12 release from dendritic cells, said dendritic cells having been isolated from a mammal within 10 hours of assaying said ability (or as assayed by one or more assays described in Section 5.6 and Section 12, infra); (c) an isolated protein that is the product of a process comprising the steps of: (1) producing a homogenate of a purified Apicomplexan organism or a cell infected with an Apicomplexan organism; (2) centrifuging the homogenate to obtain a pellet; (3) treating the pellet with a solution comprising a phospholipase; (4) centrifuging the solution to obtain a supernatant; (5) applying the supernatant to a reverse phase HPLC column; and (6) eluting bound protein from the reverse phase HPLC column, to produce eluted protein, wherein said protein has the ability (immunostimulatory ability) to stimulate interleukin-12 release from dendritic cells, said dendritic cells having been isolated from a mammal within 10 hours of assaying said ability (or as assayed by one or more assays described in Section 5.6 and Section 12, infra); (d) an isolated nucleic acid comprising a nucleotide sequence encoding any of (a)-(c), above; and (e) a cell transformed with a nucleic acid comprising a nucleotide sequence encoding any of (a)-(c) above, said nucleotide sequence being operably linked to a promoter.

The present invention also provides a method of stimulating an immune response comprising administering to a subject infected with a human immunodeficiency virus (HIV), an effective amount of a substance selected from the group consisting of: (a) an isolated protein comprising SEQ ID NO:3, 4, 6 or 7; (b) an isolated protein comprising a variant of SEQ ID NO:1 or SEQ ID NO:2, wherein said variant has only conservative amino acid substitutions relative to SEQ ID NO:1 or SEQ ID NO:2, respectively; (c) an isolated protein comprising a variant of SEQ ID NO:3, 4, 6 or 7, wherein said variant has only conservative amino acid substitutes relative to SEQ ID NO:3, 4, 6 or 7, respectively; (d) an isolated protein that has at least 25%, at least 30%, at least 35%, at least 40%, at least 45%, at least 50%, at least 55%, at least 60%, at least 65%, at least 70%, at least 75%, or at least 80% identity to the sequence of SEQ ID NO:1 or SEQ ID NO:2, as determined by a BLAST 2.0 algorithm set to default parameters; (e) an isolated protein comprising a PROF (profiling) domain; (f) an isolated Apicomplexan protein comprising an amino acid sequence encoded by a nucleic acid consisting of a nucleotide sequence hybridizable to SEQ ID NO:15, 16, 17, 18, 19, 20, or 27, or a complement of any of the foregoing SEQ ID NOs, under conditions of low stringency; (g) an isolated nucleic acid comprising a nucleotide sequence encoding any of (a)-(f) above; (h) a cell transformed with a nucleic acid comprising a nucleotide sequence encoding any of (a)-(f) above, said nucleotide sequence being operably linked to a promoter; and wherein said substance has immune stimulatory ability, e.g., has the ability to stimulate interleukin-12 release from dendritic cells, said dendritic cells having been isolated from a mammal within 10 hours of assaying said ability.

The present invention provides a method of elevating systemic level of interleukin-12 (IL-12) in a subject, wherein the subject is infected with a human immunodeficiency virus (HIV), comprising administering to said subject in whom such elevation of IL-12 level is desired an effective amount of a substance selected from the group consisting of: (a) an isolated protein comprising SEQ ID NO:1; (b) an isolated protein comprising SEQ ID NO:2; (c) an isolated protein comprising SEQ ID NO: 3, 4, 5, 6 and 7; (d) an isolated protein that is a product of process comprising the steps of: (1) producing a cell extract of Eimeria infected tissue or cells; (2) centrifuging the cell extract to produce a supernatant; (3) partitioning the supernatant by ammonium sulfate to precipitate proteins to a pellet, and resuspending the pellet into a solution; (4) applying the solution to a HIC column; (5) eluting material bound to the HIC column to produce eluted material; (6) dialyzing the eluted material to produce a sample for loading on a DEAE column; (7) applying the sample to a DEAE column; (8) eluting material bound to the DEAE column with buffer to produce an eluted fraction and concentrating the eluted fraction; (9) applying the concentrated eluted fraction to a size exclusion column; (10) collecting and pooling fractions eluted from the size exclusion column; (11) applying the pooled fractions to a HIC column to remove calcium-binding proteins; (12) applying eluted fractions from the HIC column to a HPLC HIC column and collecting active (immunostimulatory) fractions, wherein said active fractions have the ability to stimulate interleukin-12 release from dendritic cells, said dendritic cells having been isolated from a mammal within 10 hours of assaying said ability (or as assayed by one or more assays described in Section 5.6 and Section 13, infra); (13) dialyzing the active fractions; and (14) applying the dialyzed active fractions to a HPLC DEAE column and collecting one or more active (immunostimulatory) fractions, wherein said active fractions have the ability to stimulate interleukin-12 release from dendritic cells, said dendritic cells having been isolated from a mammal within 10 hours of assaying said ability (or as assayed by one or more assays described in Section 5.6 and Section 12, infra); (e) an isolated protein that is the product of a process comprising the steps of: (1) producing a homogenate of a purified Apicomplexan organism or a cell infected with an Apicomplexan organism; (2) centrifuging the homogenate to obtain a pellet; (3) treating the pellet with a solution comprising a phospholipase; (4) centrifuging the solution to obtain a supernatant; (5) applying the supernatant to a reverse phase HPLC column; and (6) eluting bound protein from the reverse phase HPLC column, to produce eluted protein, wherein said protein has the ability (immunostimulatory ability) to stimulate interleukin-12 release from dendritic cells, said dendritic cells having been isolated from a mammal within 10 hours of assaying said ability (or as assayed by one or more assays described in Section 5.6 and Section 13, infra); (f) an isolated nucleic acid comprising a nucleotide sequence encoding any of (a)-(e), above; and (g) a cell transformed with a nucleic acid comprising a nucleotide sequence encoding any of (a)-(e) above, said nucleotide sequence being operably linked to a promoter.

The present invention also provides a method of elevating systemic level of interleukin-12 (IL-12) in a subject, wherein the subject is infected with a human immunodeficiency virus (HIV), comprising administering to said subject in whom such elevation of IL-12 level is desired an effective amount of a substance selected from the group consisting of: (a) an isolated protein comprising SEQ ID NO:3, 4, 6 or 7; (b) an isolated protein comprising a variant of SEQ ID NO:1 or SEQ ID NO:2, wherein said variant has only conservative amino acid substitutions relative to SEQ ID NO:1 or SEQ ID NO:2, respectively; (c) an isolated protein comprising a variant of SEQ ID NO:3, 4, 6 or 7, wherein said variant has only conservative amino acid substitutes relative to SEQ ID NO:3, 4, 6 or 7, respectively; (d) an isolated protein that has at least 25%, at least 30%, at least 35%, at least 40%, at least 45%, at least 50%, at least 55%, at least 60%, at least 65%, at least 70%, at least 75%, or at least 80% identity to the sequence of SEQ ID NO:1 or SEQ ID NO:2, as determined by a BLAST 2.0 algorithm set to default parameters; (e) an isolated protein comprising a PROF (profiling) domain; (f) an isolated Apicomplexan protein comprising an amino acid sequence encoded by a nucleic acid consisting of a nucleotide sequence hybridizable to SEQ ID NO:15, 16, 17, 18, 19, 20, or 27, or a complement of any of the foregoing SEQ ID NOs, under conditions of low stringency; (g) an isolated nucleic acid comprising a nucleotide sequence encoding any of (a)-(f) above; (h) a cell transformed with a nucleic acid comprising a nucleotide sequence encoding any of (a)-(f) above, said nucleotide sequence being operably linked to a promoter; and wherein said substance has immune stimulatory ability, e.g., has the ability to stimulate interleukin-12 release from dendritic cells, said dendritic cells having been isolated from a mammal within 10 hours of assaying said ability.

The present invention also provides a method of treating or preventing infection with an human immunodeficiency virus (HIV), comprising adminstering to a subject in whom such treatment or prevention is desired a therapeutically or prophylactically effective amount of a purified Apicomplexan organism or a protein-containing membrane fraction of a purified Apicomplexan organism.

### 3.1 Conventions and Abbreviations

ARP Apicomplexa-related protein (the active molecule of the invention)
SIE Small intestine extract
BEX Bovine small intestine extract
GM-CSF Granulocyte macrophage—colony stimulating factor
IL2 Interleukin 2
IL12 Interleukin 12
IFNγ Interferon—gamma
DC Dendritic cell
LGL Large granular lymphocytes
NK or NK cells Natural killer cells
n_NK NK to target cell ratio, where n represents seeding ratio
CMC Cell-mediated cytotoxicity
spp. Various species of a given genus
cDNA Complementary DNA

### 4. BRIEF DESCRIPTION OF THE FIGURES

FIG. 1. Phylogenetic tree for small subunit ribosomal RNA sequences of Cyclospora and Eimeria species. Quartet puzzling maximum likelihood results are shown, with Toxoplasma gondii as the outgroup. After analysis, the outgroup branch was removed for clarity. Numbers to the left of the nodes indicate the quartet puzzling support for each internal branch. The scale bar indicates an evolutionary distance of 0.01 nucleotides per position in the sequence. Vertical distances are for clarity only. (This figure is taken from Eberhard et al., Emerging Infectios Diseases 1999, 5(5):651-658.)

FIG. 2. (A) Example of a molecular sieving chromatography step using Superdex™ 75 the activity on the NK and DC assays. (B) Typical total recovery from the purification scheme outlined in section 6, at the end of the Superdex™ size exclusion step. The activity within the crude material (corrected for total volume) is shown on the far left. The total recovered after Superdex™ chromatography is shown at the far right.

FIG. 3. (A) HIC gradient chromatography of BBX after Calcium-binding protein removal step. (B) DEAE gradient chromatography of the active fractions from the HIC gradient. (C)C8 reverse phase gradient of the active fractions from the DEAE gradient. (D) Correlation of absorbance and activity in C8 active fractions.

FIG. 4. Comparison of protein sequences from three Eimeria species. The sequences of E. tenella (EtU3) and E. acervulina (3-1E: Accession # AF113613) were obtained from cDNA translation. The cDNA for EtU3 was obtained from E. tenella mRNA. The segments from a presumed bovine Eimeria of unknown species was obtained by Edman and MS/MS amino acid sequencing of tryptic peptides.

FIG. 5. (A) Dilution profile of unpurified E. tenella extracts on the DC assay. Samples were prepared according to first extraction of section 8. Five replicate dilution curves, spanning two separate DC assays, were averaged. The error bars indicate the standard deviation of the 5 replicates. (B) C8 HPLC separation of the second E. tenella extract and the corresponding DC assay activity of the fractions.

FIG. 6: Dose/response curves for various sources of ARP. The impure E. tenella extract and the DEAE-purified recombinant sample contain ARP that is about 4% of total protein weight. The pure bovine extract and the recombinant E. tenella protein are pure but approximately 95% denatured from the reverse-phase chromatographic conditions. These estimates were used to calculate protein concentration in assays. The highly pure rARP sample was obtained from size-exclusion chromatography from the DEAE active fraction. Its purity and protein content were evaluated on a small sample by C8 reverse-phase chromatography and its activity was plotted without corrections for concentration. Response curves were normalized to similar maximal levels.

FIG. 7. (A) DNA sequence of the cDNA clone obtained from E. tenella mRNA using primers described in section 11. (B) Activity from two cultures of E. coli containing plasmids incorporating either forward or reverse orientations of the cDNA shown in (A). (C)C8 HPLC separation of the purified minimal media conditioned by E. coli containing the cloning plasmid with the cloned ARP for E. tenella, and the corresponding DC assay activity of the fractions.

FIG. 8. Correlation of induced NK-CMC with elevated levels of IFNγ in the media of treated LGL cultures. LGL/S180 co-cultures were treated with varying concentrations of BEX and analyzed for NK-CMC with the standard 4 day in vitro NK assay. LGL cultures were treated with the same concentrations of BEX and the conditioned media was analyzed for IFNγ concentration following an overnight culture.

FIG. 9. (A) Response of the DC assay to E. tenella and Bovine BBX-01c as a function of synergistic additives. In each panel, activity is measured for the antigen alone, the additives alone, the antigen with all the additives (ALL), the antigen plus a single additive (denoted with +), and the antigen with only one additive missing (denoted with a −). The two panels represent two separate assays and absolute values between the two are not comparable. (B) Loss of sensitivity of freshly isolated CD 11c⁺ dendritic cells to BBX-01c as a function of time in tissue culture conditions.

FIG. 10. DC assay activities of various bovine breeds compared to a benchmark pool. Boxes represent 25^{th} percentile (bottom) to 75^{th} percentile (top). Lines above and below boxes are the 95^{th} and 5^{th} percentile indicators, respectively. The median in denoted as a line inside the box.

FIG. 11. C8 reverse phase chromatography of ARP released from the crude membrane preparation from E. tenella oocysts after incubation in 50 mM tris-HCl pH 7.4, 1 mM PMSF at 37° C. 4 hours.

FIG. 12. Alignment of various Apicomplexa expressed sequence tag (EST) translations. Alignments were performed by visual observation. A threshold for “similarity” at a position was that no more than two amino acids that would be considered non-conservative changes relative to the other amino acids that were present at that position. If this condition is satisfied, the recorded amino acid is the one accuring most commonly in the column. Average length from start Methionine is 191. Among the ESTs compared, 41 positions (41/191=21%) have 100% identity, and 106 positions (106/191=55%) have at least 80% similarity by amino acid property.

FIG. 13. Alignment of DNA sequences of E. tenella ARP E1 clone and the EtU3 (also refered as U3 in the figure) clone.

FIG. 14. Chromatogram of the HIC gradient chromatography separation of the 80% ammonium sulfate fraction obtained from the E1 clone extract and the corresponding DC assay activity profile.

FIG. 15. Chromatogram of the DEAE gradient chromatography separation of the active fractions from the HIC chromatography of the E1 clone extract and the corresponding DC assay activity profile.

FIG. 16. Chromatogram of the Superdex isocratic chromatography separation of the active fractions from the DEAE chromatography of the E1 clone extract and the corresponding DC assay activity profile.

FIG. 17. Chromatogram of the C8 HPLC separation of a small portion of the pooled active fractions from the Superdex chromatography of the E1 clone extract. This indicates that the Superdex product is highly pure.

FIG. 18. ELISA assessment of IL 12 within the mice that were treated with an E. tenella ARP (EtU3).

FIG. 19. Alignment of DNA sequences of E. tenella ARP E1 clone and the EtU3 (also refered as U3 in the figure) clone.

FIG. 20. Deconvoluted electrospray MS spectrum showing the expected masses of 18,414 and 18,809 for the reaction of Ellman's reagent with ARP E1.

FIG. 21. Dose/response curves for both the oxidized and reduced forms of ARP. The curves are adjusted to constant protein concentration.

FIG. 22. Serum IL-12 levels in patient #5-RW, during two courses of the 5-day low-dose BBX-01c. Serum levels were measured prior to injections of BBX-01c on day 1 through day 5. A final sample was drawn either on day 9 or 10.

### 5. DETAILED DESCRIPTION OF THE INVENTION

The present invention provides compositions and methods for the prevention and treatment of an infectious disease caused by infection with HIV and for stimulating an immune response in a subject. In particular, the present invention provides Apicomlexa-related proteins (ARPs) that have immune stimulatory activity and thus have use in the treatment and prevention of an infectious disease caused by infection with HIV. Compositions comprising ARPs are provided. Methods of use of ARPs for the prevention and/or treatment of an infectious disease caused by infection with HIV, and for eliciting an immune response in a subject, are also provided. While not bound by any theory, the invention is based, in part, on the Applicants' discovery that a molecule purified from Eimeria infected bovine small intestine extracts, which is highly homologous to a surface antigen (3-1E, Lellehoj et al., 2000, Avian Diseases 44:379-389) of Apicomplexan protozoan Eimeria acervulina, is a robust activator of dendritic cells at pico- and femtomolar levels and has cross species immune stimulatory effects both in vitro and in vivo.

For clarity of disclosure, and not by way of limitation, the detailed description of the invention is divided into the following subsections:
(i) ARPs
(ii) ARPs of Purified Apicomplexa
(iii) Derivatives and Analogs of ARPs
(iv) Antibodies to ARPs, Derivatives and Analogs
(v) Structure Prediction and Functional Analysis of ARPs
(vi) Characterization and Demonstration of ARP activities
(vii) Uses of the Compositions of the Invention
(viii) Other Anti-infection Agents
(ix) Dosage Regimens
(x) Administrations, Formulations and Kits
(xi) Article of Manufacture

### 5.1 ARPs

In some embodiments, an ARP of the invention is a protein of an Apicomplexan organism that has immune stimulatory activity, as well as a fragment, a derivative, a variant, a homolog or an analog thereof. An Apicomplexan organism is one of those of the phylum Apicomplexa. Non-limiting examples of Apicomplexan organisms include but not limited to, those that are listed in Table 1, infra. In specific embodiments, an ARP of the invention includes, but is not limited to, SEQ ID NO:1 (ARP of E. tenella), SEQ ID NO:2 (ARP of E. acervulina), and a protein that comprises SEQ ID NOs: 3-7 (partial amino acid sequence of bovine Eimeria spp. ARP). SEQ ID NOs: 3-7 are not necessarily contiguous, as there may be intervening or adjacent sequences to each fragment. Preferably, a protein comprises SEQ ID NOs:3, 4, 5, 6 and 7 in an order of SEQ ID NO:3 to SEQ ID NO:7 from the N terminus to the C terminus.

In a specific embodiment, an ARP of the invention is an Apicomplexan protein (encoded by genome of an Apicomplexan organism) that has at least 25%, at least 30%, at least 35%, at least 40%, at least 45%, at least 50%, at least 55%, at least 60%, at least 65%, at least 70%, at least 75%, at least 80% or at least 85% identity to the sequence of SEQ ID NO:1 or SEQ ID NO:2 as measured by a BLAST algorithm with default parameters using the BLAST 2.0 suite of programs (Altschul et al., Nucleic Acids Res 1977, 2:3389-3402,), wherein the Apicomplexan protein has immune stimulatory and/or anti-HIV activity. In a specific embodiment, an ARP of the invention is a naturally occurring Apicomplexan protein. In some embodiments, the ARPs of the invention exist in a soluble form. In some embodiments, the ARPs of the invention exist in a membrane-linked form. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention. It is to be understood that default settings of the parameters can be readily changed as needed in the future.

In a specific embodiment, an ARP of the invention is a protein that contains a conserved PROF (profilin) domain, wherein the protein has immune stimulatory and/or anti-HIV activity. Conserved domains are defined based on recurring sequence patterns or motifs. The search for a known conserved domain can be done, e.g., at the website ncbi.nlm.nih.gov/Structure/cdd/wrpsb.cgi. In one embodiment, the conserved PROF (profilin) domain is determined by using Conserved Domain Database (CDD v. 1.60) and a RPS-BLAST (Reverse Position-Specific BLAST) algorithm set to default parameters. Current CDD database contains domains derived from two popular collections, Smart and Pfam, plus contributions from National Center for Biotechnology Information (hereinafter “NCBI”). In Conserved Domain Database, the PROF (profilin) domain is also identified as smart 00392 or cd 00148 domain, or pfam 00235 domain (PSSM Id's 14983, 14824 and 801 correspondingly). To identify conserved domains in a protein sequence, the RPS-BLAST algorithm can be used. The query sequence is compared to a position-specific score matrix prepared from the underlying conserved domain alignment. Hits may be displayed as a pairwise alignment of the query sequence with a representative domain sequence, or as a multiple alignment. See, Marchler-Bauer et al., Nucleic Acids Research 2003, 31:383-387; Marchler-Bauer et al., Nucleic Acids Research 2002, 30:281-283, all of which are incorporated herein by reference in their entirety. The “PROF” domain is represented by profilin, which is ubiquitous in nature, occurring in organisms from amoeba to mammals. Profilin is involved in the regulation of actin polymerization and may link the cytoskeleton with major signaling pathways by interacting with components of the phosphatidylinositol cycle and Ras pathway. See e.g., Korenbaum et al., Biochemistry 1998, 37(26):9274-83; Schluter et al., Biochim Biophys Acta. 1997, 1359(2):97-109, which are incorporated herein by reference in their entirety. In a specific embodiment, an ARP of the invention is a plant profilin, e.g., SEQ ID NO: 11, 12, 13 or 14.

In a specific embodiment, an ARP of the invention is an Apicomplexan protein whose encoding nucleic acid (Apicomplexan cDNA or genomic nucleic acid) hybridizes under stringent conditions (high, moderate or low stringent condition) to an ARP nucleic acid (e.g., having a sequence as set forth in SEQ ID NO:16, SEQ ID NO:17, SEQ ID NO:18, SEQ ID NO:19, or SEQ ID NO:20, or to its reverse complement, or to a nucleic acid encoding an ARP derivative, or to its reverse complement), wherein the Apicomplexan protein has immune stimulatory and/or anti-HIV activity. Stringent conditions are sequence-dependent and circumstance-dependent, for example, longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays”, 1993. See also Martin et al. (EMBO J. 1985, J 4:1625-1630) and Davies etal (Methods in Molecular Biology Vol 28: Protocols for nucleic acid analysis by non-radioactive probes, Isaac, P. G. (ed) pp 9-15, Humana Press Inc., Totowa N.J., USA), all of which are incorporated herein by reference in their entireties.

In a specific embodiment, a nucleic acid which is hybridizable to an ARP nucleic acid or its reverse complement under conditions of low stringency is provided. By way of example but not limitation, procedures using such conditions of low stringency are as follows (see also Shilo and Weinberg, Proc. Natl. Acad. Sci. U.S.A. 1981, 78, 6789-6792): filters containing DNA are pretreated for 6 hours at 40° C. in a solution containing 35% formamide, 5×SSC, 50 mM Tris-HCl (pH7.5), 5 mM EDTA, 0.1% PVP, 0.1% Ficoll, 1% BSA, and 500 μg/ml denatured salmon sperm DNA; hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used; filters are incubated in hybridization mixture for 18-20 hours at 40° C., and then washed for 1.5 hours at 55° C. in a solution containing 2×SSC, 25 mM Tris-HCl (pH7.4), 5 mM EDTA, and 0.1% SDS; the wash solution is replaced with fresh solution and incubated an additional 1.5 hours at 60° C.; filters are blotted dry and exposed for autoradiography. If necessary, filters are washed for a third time at 65-68° C. and re-exposed to film. In another example, low stringency hybridization is carried out at 62° C. without formamide. Other conditions of low stringency which may be used are well known in the art (e.g., as employed for cross-species hybridizations).

In another specific embodiment, a nucleic acid which is hybridizable to an ARP nucleic acid, or its reverse complement, under conditions of high stringency is provided. By way of example but not limitation, procedures using such conditions of high stringency are as follows: prehybridization of filters containing DNA is carried out for 8 hours to overnight at 65° C. in buffer composed of 6×SSC, 50 mM Tris-HCl (pH7.5), 1 mM EDTA, 0.02% PVP, 0.02% Ficoll, 0.02% BSA, and 500 μg/ml denatured salmon sperm DNA; filters are hybridized for 48 h at 65° C. in prehybridization mixture containing 100 μg/ml denatured salmon sperm DNA and 5-20×10⁶ cpm of ³²P-labeled probe; washing of filters is done at 37° C. for 1 hour in a solution containing 2×SSC, 0.01% PVP, 0.01% Ficoll, and 0.01% BSA; this is followed by a wash in 0.1×SSC at 50° C. for 45 min before autoradiography. In another example, high stringency hybridization is carried out at 62° C. with 50% formamide. Other conditions of high stringency which may be used are well known in the art.

In another specific embodiment, a nucleic acid which is hybridizable to an ARP nucleic acid, or its reverse complement, under conditions of moderate stringency is provided. Selection of appropriate conditions for such stringencies is well known in the art (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, © 1987-1997, Current Protocols, © 1994-1997 John Wiley and Sons, Inc.). In one non-limiting example, moderate stringency hybridization can be carried out at 62° C. with 20% formamide.

In an embodiment where a bovine ARP (which comprises SEQ ID NOs:3-7) or its derivatives are used therapeutically or prophylactic disulfide bond. In one embodiment, a bovine ARP has not been subjected to reducing conditions that would disrupt a disulfide bond. ARP protein that has been reduced has activity that is 2-5 times greater than the oxidized form.

In some embodiments, an ARP of the invention is post-translationally modified. In some embodiments, an ARP of the invention is not post-translationally modified. In specific embodiments, an ARP of the invention is glycosylated. In specific embodiments, an ARP of the invention is unglycosylated.

In some embodiments, an ARP of the invention is membrane-linked. In some embodiments, an ARP of the invention is not membrane-linked. In specific embodiments, an ARP of the invention is glysosylphosphatidylinositol (GPI)-linked. In specific embodiments, an ARP of the invention is not GPI-linked. In one embodiment, an ARP of the invention is a lipoprotein. In another embodiment, an ARP of the invention is not a lipoprotein.

In a specific embodiment, an ARP of the invention is a native protein. In a specific embodiment, an ARP of the invention is a recombinantly produced protein. In specific embodiments, an ARP of the invention has a molecular weight in the range of 18 kD to 25 kD, and an isoelectric point (pI) between 4.0 and 4.7.

Therapeutic ARPs of the invention can be tested in vitro for the desired activity by any one or more assays known in the art.

An ARP of the invention typically has potent immune stimulatory activity, even at very low concentration (e.g., pico- or femtomolar levels). In specific embodiments, a therapeutic or prophylactic composition of the invention for eliciting an immune response and for the prevention and treatment of an infectious disease caused by infection with HIV comprises an enriched ARP. As used herein, the term “enriched” in reference to a protein (e.g., a peptide, polypeptide or fusion protein) means that the protein constitutes a higher fraction of the total amount of protein present in the composition of interest, relative to the natural or original state from which the protein is derived. The enrichment can be achieved by preferential reduction in the amount of other protein present, or by a preferential increase in the amount of the specific protein of interest, or by a combination of the two. It should be noted that “enriched” does not imply that there are no other proteins present. The term also does not imply that there are no proteins present from other sources. The other source proteins may, for example, comprise protein(s) encoded by a genome of another species, or of a cloning vector. The term is meant to cover only those situations in which man has intervened to elevate the proportion of the desired protein. In specific embodiments, an ARP is greater than 0.001%, greater than 0.003%, greater than 0.01%, greater than 0.05%, greater than 0.1%, greater than 0.5%, greater than 1%, greater than 10%, greater than 20%, greater than 30% of total protein by weight.

The term “enriched” in reference to nucleic acid means that the nucleic acid constitutes a higher fraction of the total amount of nucleic acids present in the composition of interest, relative to the natural or original state from which the nucleic acid is derived. The enrichment can be achieved by preferential reduction in the amount of other nucleic acid present, or by a preferential increase in the amount of the specific nucleic acid of interest, or by a combination of the two. It should be noted that “enriched” does not imply that there are no other nucleic acids present. The term also does not imply that there are no nucleic acids present from other sources. The other source nucleic acids may, for example, comprise nucleic acid(s) encoded by a genome of another species, or of a cloning vector. The term is meant to cover only those situations in which man has intervened to elevate the proportion of the desired nucleic acid.

In a preferred embodiment, an ARP of the invention is purified. The term “purified” in reference to a protein or a nucleic acid preferably means at least one order of magnitude of purification is achieved, more preferably two or three orders of magnitude, most preferably four or five orders of magnitude of purification of the starting material or of the natural material. Thus, the term “purified” as used herein does not mean that the material is 100% purified and thus does not mean that a purified protein or a nucleic acid excludes any other material. In specific embodiments, a purified ARP is at least 60%, at least 80%, or at least 90% of total protein or nucleic acid, as the case may be, by weight. In a specific embodiment, a purified ARP is purified to homogeneity as assayed by, e.g., sodium dodecyl sulfate polyacrylamide gel electrophoresis, or agarose gel electrophoresis.

As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane component). A material shall be deemed isolated if it is present in a cell extract or if it is present in a heterologous cell or cell extract. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined or proximal to non-coding regions (but may be joined to its native regulatory regions or portions thereof), or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like, i.e., when it forms part of a chimeric recombinant nucleic acid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

In a specific embodiment, an ARP of the invention is present in the form of purified viable or inactivated Apicomplexan organisms at any developmental stage or a protein fraction thereof (e.g., a protein-containing membrane preparation thereof, or a storage granule preparation thereof). In a specific embodiment, an Apicomplexan organism is an Eimeria, which is species-specific and usually cannot cause symptomatic infection in a host of a different species from its native host. In another specific embodiment, an inactivated Apicomplexan organism is a life cycle defective Apicomplexan organism.

### 5.1.1 Preparation and Purification of ARP

In some embodiments, the compositions of the invention that are used in prevention or treatment of infection with HIV comprise an enriched, an isolated, or a purified ARP. In accordance with the methods described herein, an ARP employed in a composition of the invention can be in the range of 0.001 to 100 percent of the total mg protein, or at least 0.001%, at least 0.003%, at least 0.01%, at least 0.1%, at least 1%, at least 10%, at least 30%, at least 60%, or at least 90% of the total mg protein. In one embodiment, an ARP employed in a composition of the invention is at least 4% of the total protein. In another embodiment, an ARP is purified to apparent homogeneity, as assayed, e.g., by sodium dodecyl sulfate polyacrylamide gel electrophoresis. The ARPs of the invention can be purifed, e.g., from the homogenates of infected tissue or a purified Apicomplexan organism with the use of either soluble fractions (supernatants) or insolubles (pellets), or from intact cells (e.g., mammalian cells expressing a recombinant protein or Apicomplexa cells) with the use of enzymes acting on cell surface. Any techniques known in the art can be used in purifying an ARP, including but are not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See Scopes, PROTEIN PURIFICATION, PRINCIPLES AND PRACTICE, 3^{rd} ed., Springer (1994), the entire text is incorporated herein by reference. The present invention also encompasses any modification to the methods of isolation of ARP that affects ARP activity and/or aggregation state through regulation of binding of divalent cations. The purity of ARP can be assayed by any methods known in the art, such as but not limited to, gel electrophoresis. See Scopes, supra.

### 5.1.1.1 Purification of Soluble ARPs from Tissue and Cell Extracts

In one embodiment, an ARP is purified from Apicomplexa infected tissue of a vertebrate animal (the animal may have symptomatic infection or non-symptomatic infection), including but not limited to, a cow, a sheep, a pig, a goat, a horse, a dog, a cat, a rodent, an avian, a primate (e.g., monkey), a mammal, and a human. In another embodiment, an ARP is purified from a cell culture infected with an Apicomplexan organism. In yet another embodiment, an ARP is purified from a preparation of an Apicomplexan organism. A procedure that can be used to purify ARPs from infected tissues, a cell culture, or a preparation of Apicomplexan organism, is presented by way of example but not limitation in Section 6, infra.

Briefly, by way of example but not limitation, a preparation of infected tissue or cells (e.g., a homogenized small intestine tissue or a preparation of the cells of intestinal mucosa) is centrifuged. The resulting liquid supernatants are brought to 45% saturation with solid ammonium sulfate, centrifuged and the pellet is discarded. The supernatants are brought to 80% saturation using solid ammonium sulfate, centrifuged again, and the supernatant is discarded. The pellets are dissolved in 1.5 M Ammonium Sulfate/50 mM Sodium Phosphate (pH 6.8) after resuspending completely within the bottles or tubes. Non-dissolved material is removed by centrifugation.

In a preferred embodiment, to reduce the macromolecular load prior to size-exclusion chromatography, two bulk processing steps are used. The first step uses a HIC column. A large amount of inactive material, particularly polynucleic acid, is removed in the flow-through and wash. The active (immunostimulatory) material (wherein said active fractions have the ability to stimulate interleukin-12 release from dentritic cells, said dentritic cells having been isolated from a mammal within 10 hours of assaying said ability (or assayed by one or more assays described in Section 5.6 and Section 13, infra)) is eluted after a change to a buffer free of ammonium sulfate. The eluted material is prepared for DEAE chromatography by dialysis against 4 L of 0.2M NaCl/10 mM Sodium Phosphate (pH7.4) at 4° C. with buffer exchanges. The elution is processed in a similar fashion on DEAE ion exchange chromatography where the flow-through and wash are discarded. The elution at higher NaCl concentration is retained, concentrated on a membrane pressure cell, and added to a size exclusion column (e.g., a Superdex™ column). Specific activity (with respect to protein content, as assayed by one or more assays described in Section 5.6 and Section 13) is determined on the retained fractions from each step in the purification. Approximately 7000-fold purification is reached at the end of the size exclusion chromatography step. In specific embodiments (e.g., when small intestine extracts are used, bacterial endotoxin is always a contaminant), the endotoxin levels are monitored with the limulus amebocyte lysate (hereinafter “LAL”) assay at each step in the purification. Endotoxin levels are reduced approximately 1000-fold resulting in final levels that are well below those that can interfere with the DC assay (threshold of 1000 U/mL for minimal DC activity. See Section 5.6 and Section 13 for detailed description of the DC assay).

In some embodiments, to obtain enough material for final purification, pools are created that contain the extracts from multiple samples (e.g., animal intestines). Preferably, these are pre-selected for high activity in the crude supernatants (for an example of such pre-testing of the activity, see Section 6.2.1, infra). Several batches of intestinal or cell preparations are combined, concentrated, cleared of calcium binding proteins (by an HIC column, see Section 6.2.2., infra) and designated as a pool.

To obtain a purified product, a pool is initially separated on a HPLC HIC column. The active fractions (wherein said active fractions have the ability to stimulate interleukin-12 release from dendritic cells, said dentritic cells having been isolated from a mammal within 10 hours of assaying said ability (or as assayed by one or more assays described in Section 5.6 and Section 13, infra) are dialyzed to remove ammonium sulfate and are applied to an HPLC DEAE column in 0.15M NaCl. A NaCl gradient is applied and the activity (immunostimulatory activity, as assayed by one or more assays described in Section 5.6 and Section 13, infra) eluted with a major peak near 0.3M ionic strength. Preferably, DEAE runs are used to process the active material from the HIC. In specific embodiments, the pooled active region is applied to a reverse phase HPLC column (e.g., C8 reverse phase HPLC column) and eluted with an aqueous-acetonitile gradient. The activity (immunostimulatory activity, as assayed by one or more assays described in Section 5.6 and Section 13) eluted near 40% acetonitrile on a slow, shallow gradient elution protocol. The question of purity of these active fractions is addressed by plotting activity versus absorbance along a finely-sampled separation. A reasonably robust linear regression line and the intersection near zero indicate the lack of contaminating molecules.

The reverse phase HPLC column purification step denatures most of the protein. In a preferred embodiment, the eluted protein from a reverse phase HPLC column is renatured by a method known in the art. In another embodiment, reverse phase HPLC column is not used to purify an ARP of the invention.

In some embodiments, a soluble ARP can be isolated by a process comprising the steps of: (1) producing a solution with enriched ARP, e.g., an extract of cells expressing a recombinant ARP protein; (2) centrifuging the solution to obtain a supernatant; (3) partitioning the supernatant by ammonium sulfate to precipitate proteins, centrifuging the precipitate to obtain a pellet, and resuspending the pellet into a solution; (4) applying the solution to a low pressure HIC column and collecting active (immunostimulatory) fractions (wherein said active fractions have the ability to stimulate interleukin-12 release from dentritic cells, said dendritic cells having been isolated from a mammal within 10 hours of assaying said ability (or as assayed by one or more assays described in Section 5.6 and Section 13, infra)); (5) dialyzing the active fractions; (6) applying the dialyzed active fractions to HPLC DEAE column and collecting one or more active (immunostimulatory) fractions (wherein said active fractions have the ability to stimulate interleukin-12 release from dendritic cells; said dendritic cells having been isolated from a mammal within 10 hours of assaying said ability (or assayed by one or more assays described in Section 5.6 and Section 13, infra)) and (7) applying the active fractions to a size exclusion column and collect eluted active (immunostimulatory) fractions (wherein said active fractions have the ability to stimulate interleukin-12 release from dendritic cells; said dendritic cells having been isolated from a mammal within 10 hours of assaying said ability (or assayed by one or more assays described in Section 5.6 and Section 13, infra) (see Section 10.2, infra)).

In a specific embodiment, a soluble ARP is isolated by a process comprising the steps of: (1) producing mineral media spent by bacteria expressing a recombinant ARP; (2) applying the media to a DEAE column; (3) eluting material bound to the DEAE column with buffer to produce an eluted fraction; (4) applying the eluted fraction to a size exclusion column; and (5) collecting the fractions eluted from the size exclusion column.

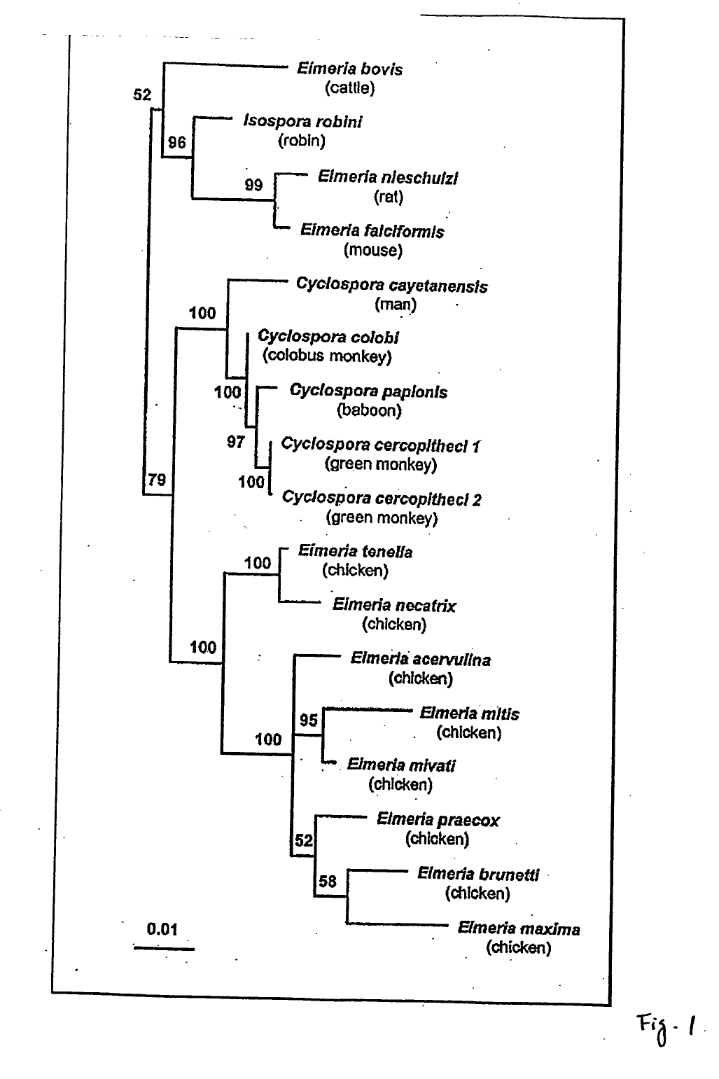

In another embodiment, an ARP is purified from an Apicomplexan protozoan, including but is not limited to, those that are listed in Table 1. FIG. 1 also provides phylogenetic information on some of the Apicomplexan protozoa. A non-limiting example of the purification process is given in Section 8.

TABLE 1
Apicomplexa

Coccidia

Eimeriida

Cryptosporidiidae

Cryptosporidium andersoni
Cryptosporidium baileyi
Cryptosporidium canis
Cryptosporidium felis
Cryptosporidium galli
Cryptosporidium meleagridis
Cryptosporidium muris
Cryptosporidium parvum
Cryptosporidium saurophilum
Cryptosporidium serpentis
Cryptosporidium wrairi
Cryptosporidium sp.
unclassified Cryptosporidium

Eimeriidae

Caryospora

Caryospora bigenetica
Caryospora sp.

Choleoeimeria

Choleoeimeria sp.

Cyclospora

Cyclospora cayetanensis
Cyclospora cercopitheci
Cyclospora colobi
Cyclospora papionis
Cyclospora sp.
Cyclospora sp. strain Gombe 22
Cyclospora sp. strain Gombe 34
Cyclospora sp. strain Gombe 40

Eimeria

Eimeria acervulina
Eimeria adeneodei
Eimeria ahsata
Eimeria alabamensis
Eimeria albigulae
Eimeria antrozoi
Eimeria arizonensis
Eimeria aubernensis
Eimeria bovis
Eimeria brunetti
Eimeria catronensis
Eimeria chaetodipi
Eimeria chobotari
Eimeria crandallis
Eimeria dipodomysis
Eimeria falciformis
Eimeria faurei
Eimeria langebarteli
Eimeria leucopi
Eimeria maxima
Eimeria meleagrimitis
Eimeria mitis
Eimeria mivati
Eimeria necatrix
Eimeria nieschulzi
Eimeria onychomysis
Eimeria ovinoidalis
Eimeria papillata
Eimeria peromysci
Eimeria pilarensis
Eimeria polita
Eimeria porci
Eimeria praecox
Eimeria reedi
Eimeria rioarribaensis
Eimeria scabra
Eimeria scholtysecki
Eimeria separata
Eimeria sevilletensis
Eimeria stiedae
Eimeria telekii
Eimeria tenella
Eimeria tropidura
Eimeria weybridgensis

Goussia

Goussia janae

Isospora

Isospora belli
Isospora felis
Isospora gryphoni
Isospora insularius
Isospora ohioensis
Isospora peromysis
Isospora robini
Isospora suis

Sarcocystidae

Toxoplasma

Eucoccidiorida

Adeleorina

Lankesterellidae

Lankesterella

Colpodellidae

Colpodella

Colpodella pontica
Colpodella sp. ATCC50594

Gregarinia

Eugregarinida

Gregarinidae
Lecudinidae
Leidyanidae
Monocystidae
Ophryocystidae

Pseudomonocystis

Pseudomonocystis lepidiota

Haemosporida (haemosporidians)

Haemoproteus

Haemoproteus chelodinae
Haemoproteus columbae
Haemoproteus kopki
Haemoproteus majoris
Haemoproteus ptyodactylii
Haemoproteus sylvae
Haemoproteus sp.
unclassified Haemoproteus

Hepatocystis

Hepatocystis sp.

Leucocytozoon

Leucocytozoon dubreuli
Leucocytozoon simondi

Plasmodium

Plasmodium agamae
Plasmodium atheruri
Plasmodium azurophilum
Plasmodium berghei
Plasmodium brasilianum
Plasmodium chabaudi
Plasmodium chiricahuae
Plasmodium cuculus
Plasmodium cynomolgi
Plasmodium elongatum
Plasmodium fairchildi
Plasmodium falciparam
(malaria parasite P. falciparum)
Plasmodium fieldi
Plasmodium floridense
Plasmodium fragile
Plasmodium gallinaceum
Plasmodium giganteum
Plasmodium gonderi
Plasmodium guanggong
Plasmodium heteronucleare
Plasmodium hylobati
Plasmodium inui
Plasmodium juxtanucleare
Plasmodium knowlesi
Plasmodium lophurae
Plasmodium malariae
Plasmodium cf. malariae
Plasmodium mexicanum
Plasmodium nucleophilum
Plasmodium ovale (malaria parasite P. ovale)
Plasmodium reichenowi
Plasmodium relictum
Plasmodium rouxi
Plasmodium simiovale
Plasmodium simium
Plasmodium vinckei
Plasmodium vivax (malaria parasite P. vivax)
Plasmodium yoelii
Plasmodium sp.
unclassified Plasmodium

unidentified Haemosporida

Piroplasmida (Piroplasmids)

Babesiidae

Babesia

Theileriidae

Cytauxzoon
Theileria
Theileria-related sp.

unclassified Piroplasmida

Piroplasmida gen. sp. BH1
Piroplasmida gen. sp. BH3
Piroplasmida gen. sp. CA1
Piroplasmida gen. sp. CA3
Piroplasmida gen. sp. CA4
Piroplasmida gen. sp. FD1
Piroplasmida gen. sp. MD1
Piroplasmida gen. sp. WA1
Piroplasmida gen. sp. WA2

unclassified Apicomplexa

Tridacna hemolymph apicomplexan
Apicomplexa sp. 72141
unidentified symbiont Type N

For more information, see e.g., the website at ncbi.nlm.nih.gov/Taxonomy/taxonomyhome.html/index.cgi, Plasmodium falciparum, Apicomplexa; the website at plasmodb.org; or the website at biology.unm.edu/biology/coccidia.

A purified Apicomplexan organism can be obtained by any method known in the art. See e.g., Tomley, Methods: A Companion to Methods in Enzymology 1997, 13:171-176; Ryley et al., Parasitology 1976, 73:311-362; U.S. Pat. Nos. 6,001,363; 6,100,241; 5,792,644; 6,451,984; and 6,008,342. For a detailed example, also see Section 8.

In one embodiment, ARP is purified from the class coccidian. In another embodiment, ARP is purified from the family Eimeriidae. In yet another embodiment, ARP is purified from the genus Eimeria.

Briefly, by way of example but not limitation, ARP can be purified from E. tenella as follows: sporulated oocysts are suspended in PBS and centrifuged. The supernatant is removed and retained. The pellet is suspended in 30 mM Hepes and homogenized on ice. This processing will result in most oocysts to be broken, releasing sporocysts that remain largely intact. The homogenate is transferred to a microcentrifuge tube and centrifuged.

In a second preparation, the homogenate is sonicated with care taken to keep the homogenate cooled during the sonication. Approximately 80% of the released sporocysts are disrupted. The suspensions are pooled and microcentrifuged. The pellet is retained and stored at 4° C. for future processing.

The starting material for the 3rd extract of sporulated oocysts is the residual pellet of the 2nd extract preparation. The pellet material is suspended in Hepes and transferred to a beadbeater tube containing glass beads. The tube is then completely filled with Hepes and processed in a Mini-Beadbeater with care taken to prevent excessive heating. The suspension is microcentrifuged briefly and the supernatant is separated from the glass beads. This supernatant is microcentrifuged and is filter-sterilized and used for purification of ARP by a reverse phase HPLC chromatography (e.g., C8 reverse phase HPLC chromatography). The pellet can be used for purification of the membrane-linked form of ARP.

### 5.1.1.2 Purification of Membrane-Linked ARPs

In a specific embodiment, the therapeutic or prophylactic compositions of the invention comprise a protein-containing membrane preparation of Apicomplexan protozoa. While not bound by any theory, the ARP is believed to exist in two forms: a soluble form and a membrane-linked form. In a specific embodiment, an ARP of the invention is linked to a cell membrane through a glycosylphosphatidylinositol (GPI) anchor (for description of protozoan-derived GPI and its receptor (Toll-like receptor 2) see, e.g., Almeida et al., J. Leukocyte Biology 2001, 70:467-477; Campos et al., the J. Immunol. 2001, 167:416-423; Ropert et al., Current Opinion in Microbiology 2000, 3:395-403). When an Apicomplexa organism infects a host, such as a cow, soluble ARP may also originate from the membranes of the Apicomplexa organism due to cleavage by endogenous phopholipase C or D present in the host's blood. An ARP can be extracted from a membrane by a non-enzymatic or an enzymatic method. In a specific embodiment, an ARP is extracted from a membrane by a detergent. In another specific embodiement, an ARP is released from a membrane by an enzymatic method, e.g., using internal and external phospholipase, or a protease. Co-sedimenting large ARP aggregates (if any) are dissolved simultaneously if the incubation buffer is free of divalent cations.

In certain embodiments, a membrane-bound ARP is isolated by a process comprising the steps of: (1) producing a homogenate of a purified Apicomplexan organism, cells infected with an Apicomplexan organism or cells expressing a recombinant ARP; (2) centrifuging the homogenate to obtain a pellet; (3) suspending the pellet in a solution and treating the solution to release the ARP by using a non-enzymatic method or an enzymatic method; (4) centrifuging the solution to obtain a supernatant highly enriched in ARP; (5) applying the supernatant to a reverse phase HPLC column; and (6) eluting bound protein from the reverse phase HPLC column. In specific embodiments, the eluted bound protein of step (6) is renatured by any technique known in the art. The immunostimulatory activity of an ARP is monitored by one or more assays described in Section 5.6 and Section 13 during the isolation process. In a specific embodiment, a membrane-bound ARP is prepared by the following method:

Briefly, by way of example but not limitation, crude membrane preparation from the sporozoites of E. tenella can be prepared as follows: a suspension of oocysts in homogenization buffer (hereinafter “HB”) (50 mM Tris pH 7.4, 1 mM PMSF, 50 μl/ml of protease inhibitors cocktail) is sonicated on ice followed by freeze-thaw cycles (liquid nitrogen/room temperature). This procedure results in almost complete breakage of oocysts, sporocysts and sporozoites. 10 mM ZnCl₂ can be added to inhibit the release of an ARP from membranes by internal phospholipase and/or facilitate ARP aggregation to increase the yield.

The cell homogenate is then centrifuged to pellet membranes. This centrifugation regime also pellets nuclei, other organelles, pelletable ARP aggregates and storage granules, if any. Organelles can be disrupted in further purification cycles in HB without ZnCl₂.

Pellet is suspended in 1 ml of HB and centrifuged again. Centrifugation/resuspension is repeated once more with and 2-3 times more without ZnCl₂ in the suspension buffer. Supernatants from centrifugation cycles done without ZnCl₂ contain significant amounts of ARP.

The final pellet is suspended in 100 μl of HB with or without protease inhibitors and incubated at 37° C. 1-4 hrs to release additional quantities of ARP. Phospholipase C at 16 U/ml facilitates the ARP release. The ARP released into solution can be separated from the insoluble material by centrifugation, followed by two washing cycles. All three supernatants are combined. They contain ARP with small amount of contaminants which can be removed by a single separation by reverse phase chromatography (e.g., C8 reverse phase chromatography).

### 5.1.1.3 Antibody-Affinity Purification of ARP

In another embodiment, antibody is used to purify an ARP or a fragment thereof. Any ARP preparations, e.g., an Apicomplexa infected tissue extract, cell culture, or a protein containing membrane preparation, can be used in antibody affinity purification of ARP. Antibodies can also be used in purifying a recombinant ARP product or a synthesized ARP. Antibodies can be monoclonal or polyclonal, and antibodies can be raised against the ARP or against a fragment of ARP (e.g., comprising a hydrophilic portion). In a specific embodiment, an antibody that binds to SEQ ID NO:26 (bovine ARP fragment 1 (SEQ ID NO:3) fused to bovine ARP fragment 2 (SEQ ID NO:4) fused to bovine ARP fragment 3 (SEQ ID NO:5) fused to bovine ARP fragment 4 (SEQ ID NO:6) and fused to bovine ARP fragment 5 (SEQ ID NO:7)) is used to purify an ARP or a fragment thereof. When ARP is engineered to express as a fusion protein, antibodies raised against a portion of the fused protein that is not ARP can be used to purify the ARP. Antibodies can be produced by any method known in the art or as described in Section 5.4., infra.

Methods of using antibodies to purify a protein are standard and well known in the art, and are not described in detail here. See, e.g., Scopes, PROTEIN PURIFICATION, PRINCIPLES AND PRACTICE, 3^{rd} ed., Springer (1994), at pp 187-236, the entire text of which is incorporated herein by reference.

### 5.1.2 Recombinant Expression of ARP

Methods known in the art can be utilized to recombinantly produce ARP. A nucleic acid sequence encoding ARP can be inserted into an expression vector for propagation and expression in host cells.

An expression construct, as used herein, refers to a nucleotide sequence encoding ARP or a fragment thereof operably associated with one or more regulatory regions which enable expression of ARP in an appropriate host cell. “Operably-associated” refers to an association in which the regulatory regions and the ARP sequence to be expressed are joined and positioned in such a way as to permit transcription, and ultimately, translation.

The regulatory regions necessary for transcription of the ARP can be provided by the expression vector. A translation initiation codon (ATG) may also be provided if the ARP gene sequence lacking its cognate initiation codon is to be expressed. In a compatible host-construct system, cellular transcriptional factors, such as RNA polymerase, will bind to the regulatory regions on the expression construct to effect transcription of the modified ARP sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to the ARP gene sequence or to insert the ARP gene sequence into the cloning site of a vector, linkers or adapters providing the appropriate compatible restriction sites may be ligated to the ends of the cDNAs by techniques well known in the art (Wu et al., Methods in Enzymol 1987, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising an ARP sequence operably associated with regulatory regions can be directly introduced into appropriate host cells for expression and production of ARP without further cloning. See, e.g., U.S. Pat. No. 5,580,859. The expression constructs can also contain DNA sequences that facilitate integration of the ARP sequence into the genome of the host cell, e.g., via homologous recombination. In this instance, it is not necessary to employ an expression vector comprising a replication origin suitable for appropriate host cells in order to propagate and express ARP in the host cells.

A variety of expression vectors may be used including, but not limited to, plasmids, cosmids, phage, phagemids or modified viruses. Such host-expression systems represent vehicles by which the coding sequences of interest may be produced and subsequently purified, but also represent cells which may, when transformed or transfected with the appropriate nucleotide coding sequences, express ARP in situ. These include, but are not limited to, microorganisms such as bacteria (e.g., E. coli and B. subtilis) transformed with recombinant bacteriophage DNA, plasmid DNA or cosmid DNA expression vectors containing ARP coding sequences; yeast (e.g., Saccharomyces, Pichia) transformed with recombinant yeast expression vectors containing ARP coding sequences; insect cell systems infected with recombinant virus expression vectors (e.g., baculovirus) containing ARP coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing ARP coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells, especially for the expression of whole recombinant ARP molecule, are used for the expression of a recombinant ARP molecule. For example, mammalian cells such as Chinese hamster ovary cells (CHO) can be used with a vector bearing promoter element from major intermediate early gene of cytomegalocirus for effective expression of ARPs (Foecking et al., Gene 1986, 45:101; and Cockett et al., Bio/Technology 1990, 8:2).

In bacterial systems, a number of expression vectors may be advantageously selected depending upon the use intended for the ARP molecule being expressed. For example, when a large quantity of such an ARP is to be produced, for the generation of pharmaceutical compositions of an ARP molecule, vectors which direct the expression of high levels of fusion protein products that are readily purified may be desirable. Such vectors include, but are not limited to, the E. coli expression vector pCR2.1 TOPO (Invitrogen), in which the ARP coding sequence may be directly ligated from PCR reaction and may be placed in frame to the lac Z coding region so that a fusion protein is produced; pIN vectors (Inouye & Inouye, Nucleic Acids Res. 1985, 13:3101-3109; Van Heeke & Schuster, J. Biol. Chem. 1989, 24:5503-5509) and the like. Series of vectors like pFLAG (Sigma), pMAL (NEB), and pET (Novagen) may also be used to express the foreign polypeptides as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific protease like enterokinase allows to cleave off the APR. The pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. The ARP coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the ARP coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing ARP in infected hosts (see, e.g., Logan & Shenk, Proc. Natl. Acad. Sci. USA 1984, 8(1):355-359). Specific initiation signals may also be required for efficient translation of inserted ARP coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 1987, 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7030 and HsS78Bst cells. Expression in a bacterial or yeast system can be used if post-translational modifications turn to be non-essential for the activity of ARP.

For long term, high yield production of properly processed ARP, stable expression in cells is preferred. Cell lines that stably express ARP may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while ARP is expressed continuously.

A number of selection systems may be used, including but not limited to, antibiotic resistance (markers like Neo, which confers resistance to geneticine, or G-418 (Wu and Wu, Biotherapy 1991, 3:87-95; Tolstoshev, Ann. Rev. Pharmacol. Toxicol. 1993, 32:573-596; Mulligan, Science 1993, 260:926-932; and Morgan and Anderson, Ann. Rev. Biochem. 1993, 62: 191-217; May, 1993, TIB TECH 11(5):155-2 15); Zeo, for resistance to Zeocin; Bsd, for resistance to blasticidin, etc.); antimetabolite resistance (markers like Dhfr, which confers resistance to methotrexate, Wigler et al., Natl. Acad. Sci. USA 1980, 77:357; O'Hare et al., Proc. Natl. Acad. Sci. USA 1981, 78:1527); gpt, which confers resistance to mycophenolic acid (Mulligan & Berg, Proc. Natl. Acad. Sci. USA 1981, 78:2072); and hygro, which confers resistance to hygromycin (Santerre et al., Gene 1984, 30:147). In addition, mutant cell lines including, but not limited to, tk-, hgprt- or aprt-cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of ARP. Modified culture conditions and media may also be used to enhance production of ARP. Any techniques known in the art may be applied to establish the optimal conditions for producing ARP.

### 5.1.2.1 Isolation of ARP Gene

The gene encoding an ARP can be isolated by any technique known in the art. Specific embodiments for the isolation of an ARP gene follow. For example, a protozoa of genus Apicomplexa can be used to isolate its total RNA. Once the total RNA of an Apicomplexa protozoan is available, the ARP gene can be isolated by methods known in the art, including but are not limited to, screening of a cDNA library, affinity purification of ARP mRNA, and polymerase chain reaction (PCR).

In specific embodiments, an isolated ARP gene comprises a nucleic acid molecule encoding SEQ ID NO:1 (ARP of E. tenella), SEQ ID NO:2 (ARP of E. acervulina), or SEQ ID NOs: 3-7 (partial sequences of bovine Eimeria spp. ARP. SEQ ID NOs:3-7 are not necessarily contiguous, there may be intervening or adjacent sequences to each fragment, though preferably the nucleic acid molecule encods a protein comprising SEQ ID NOs:3, 4, 5, 6 and 7 in an order of SEQ ID NO:3 to SEQ ID NO:7 from the N-terminus to the C-terminus). In specific embodiments, an isolated ARP gene consists a nucleic acid molecule encoding SEQ ID NO:1, SEQ ID NO:2, or SEQ ID NO:26 (SEQ ID NO:3 fused to SEQ ID NO:4 fused to SEQ ID NO:5 fused to SEQ ID NO:6 and fused to SEQ ID NO:7).

In one embodiment, cDNA library is generated by using any method known in the art (e.g., OrientExpress cDNA Library Construction System (Novagen)). Total mRNA is used to synthesize the first strand, second strand, and ligated into lambda (λ) bacteriophage arms. The constructed phage DNA can be packaged into bacteriophage by, e.g., PhageMaker Kit (Novagen). The bacteriophages containing different DNA inserts are used to infect the bacterial host to generate the lambda bacteriophage cDNA library. Normally, the library will contain 10⁵ to 10⁶ primary clones. The phage library are amplified and plated out to e.g., about 10⁴ clones per plate. The library can then be screened by membrane lifting technique, using an ARP gene probe (e.g., an E. tenella ARP). Alternatively, the cDNA library can be screened by induction of protein expression by isopropyl-1-thio-β-D-galactoside (IPTG). The membranes lifted from the plates contained the expressed proteins are screened by using antibody generated against an ARP (e.g., SEQ ID NO: 1 or 2). The identified clones are analyzed by DNA sequencing. The correct DNA insert are subcloned into protein expression vector for protein expression. The recombinant protein can be tested for DC activating property.

In another embodiment, an ARP gene (e.g., SEQ ID NO:15, 16, 17, 18, 19, 20, or 27) is cloned into a vector (e.g., pET-Blue2 vector (Novagen)) in the inverse orientation. This plasmid is transformed into a bacterial host. The bacterial strain harboring the pET-Blue2 plasmid has the ability to generate single strand (ss) phage DNA after infection with the helper phage R408. The positive strand of the insert will be made during the phage formation. The positive strand with the inverse orientation would correspond to the negative strand of the original ARP gene cloned into the vector. This ssDNA will be complimentary to a targeted ARP mRNA. The ssDNA can be biotinylated and added to the total Apicomplexan protozoan mRNA preparation to allow hybridization of the two complimentary strands. The complex can be isolated by binding to streptavidin magnetic beads. After washing of the unbound mRNA, the bound targeted Apicomplexan ARP mRNA will be eluted under low salt condition, while the biotinylated ssDNA will remain bound to the magnetic bead through strong biotin-streptavidin binding. The resulting mRNA, which is specific for targeted Apicomplexan ARP gene, will be used for synthesizing first strand DNA, second strand DNA, and cloned into protein expression vector for DNA sequencing and protein expression. DNA sequence is confirmed by homology to an ARP gene. The recombinant protein is expressed to test DC activation function.

In another embodiment, using the total mRNA isolated from an Apicomplexa protozoan, the first strand can be synthesized by MMLV reverse transcriptase using oligo dT as primer. The resulting sample will be used to perform polymerase chain reaction (PCR) by using Taq polymerase and specific primers determined by an ARP gene sequence (e.g., SEQ ID NOs: 15, 16, 17, 18, 19, 20, or 27) or consensus of ESTs from GenBank. PCR products can be analyzed by DNA agarose gel to determine the molecular weight as compared to molecular weight standard and the expected value from an ARP gene. PCR products can be introduced into cloning vector (e.g., pCR 2.1 TOPO from Invitrogen) and the plasmids with the correct insert can be selected on a basis of analysis of their size, and PCR amplification pattern with primers specific to the ARP gene. Finally the gene can be re-cloned into an expression vector. The cloned gene can be subjected to DNA sequence analysis for final confirmation of ARP sequence and precise determination of the ARP gene.

In another embodiment, polymerase chain reaction (PCR) is used to amplify the desired sequence in a genomic or cDNA library, prior to selection. Oligonucleotide primers representing an ARP sequences can be used as primers in PCR. In a preferred aspect, the oligonucleotide primers represent at least part of conserved segments of strong homology between ARP genes of different species. The synthetic oligonucleotides may be utilized as primers to amplify sequences from a source (RNA or DNA), preferably a cDNA library, of potential interest. PCR can be carried out, e.g., by use of a Perkin-Elmer Cetus thermal cycler and Taq polymerase (e.g., Gene Amp™). The nucleic acid being amplified can include mRNA or cDNA or genomic DNA from any species. One may synthesize degenerate primers for amplifying homologs from other species in the PCR reactions.

It is also possible to vary the stringency of hybridization conditions used in priming the PCR reactions, to allow for greater or lesser degrees of nucleotide sequence similarity between the ARP nucleotide sequences and a nucleic acid homolog being isolated. For cross species hybridization, low stringency conditions are preferred. For same species hybridization, moderately stringent conditions are preferred. After successful amplification of a segment of an ARP homolog, that segment may be cloned and sequenced by standard techniques, and utilized as a probe to isolate a complete cDNA or genomic clone. This, in turn, permits the determination of the gene's complete nucleotide sequence, the analysis of its expression, and the production of its protein product for functional analysis, as described below. In this fashion, additional genes encoding ARPs and ARP analogs may be identified.

### 5.1.3 Peptide Synthesis

An alternative to producing ARP or a fragment thereof by recombinant techniques is peptide synthesis. For example, an entire ARP, or a peptide corresponding to a portion of ARP can be synthesized by use of a peptide synthesizer. Conventional peptide synthesis or other synthetic protocols well known in the art may be used.

Peptides having the amino acid sequence of ARP or a portion thereof may be synthesized by solid-phase peptide synthesis using procedures similar to those described by Merrifield, J. Am. Chem. Soc. 1963, 85:2149. During synthesis, N-α-protected amino acids having protected side chains are added stepwise to a growing polypeptide chain linked by its C-terminal and to an insoluble polymeric support, i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting ARP or a fragment thereof is accomplished using conventional procedures, such as preparative HPLC using gel permeation, partition and/or ion exchange chromatography. The choice of appropriate matrices and buffers are well known in the art and so are not described in detail herein.

### 5.2 ARPs of Purified Apicomplexa

In specific embodiments, the present invention provides a purified Apicomplexan organism that contains an ARP for the treatment of an infectious disease caused by infection with HIV, and for eliciting an immune response. In a preferred embodiment, the purified Apicomplexan organism is also an inactivated Apicomplexan organism or an Apicomplexan organism that can not cause the disease in an intended host. A purified or an inactivated Apicomplexan organism containing ARP can be directly administered, preferably orally, to a subject for treatment or prevention of an infectious disease caused by infection with HIV.

Some genus of Apicomplexa, such as Eimeria, is highly species-specific. For example, an Eimeria that infect avian species usually does not cause symptomatic infections in non-avian species. In one embodiment of the invention, an Apicomplexan organism containing ARP is adminitered to a subject that is not a natural host of the organism for the treatment and prevention of an infectious disease caused with infection with HIV.

In a preferred embodiment, Eimeria sporulated oocysts containing ARPs are administered to a subject that is not a natural host of the organism for the treatment or prevention of an infectious disease caused by infection with HIV.

In another embodiment of the invention, an Apicomplexan organism containing ARP is engineered to have a defective life cycle so that it cannot cause a symptomatic infection in a host, and the engineered Apicomplexan organism is administered to a subject for treatment or prevention of an infectious disease caused by infection with HIV. Any technique known in the art that is able to cause an Apicomplexan organism to have a defective life cycle can be used. Such techniques include but not limited to, genetic engineering, use of chemical toxins, or radiation. Whether an Apicomplexan organism is having a defective life cycle can be tested in animals by infection followed by biopsy to verify that the organism has entered cells. An appropriate waiting period would indicate if the organism is unable to complete its life cycle and create oocysts in the feces.

Apicomplexa have complex life cycles, and there is much variation among different apicomplexa groups. Both asexual and sexual reproduction are involved, although some apicomplexa skip one or the other stage. The basic life cycle may be said to start when an infective stage, or sporozoite, enters a host cell, and then divides repeatedly to form numerous merozoites. Some of the merozoites transform into sexually reproductive cells, or gamonts. Gamonts join together in pairs and form a gamontocyst. Within the gamontocyst, the gamonts divide to form numerous gametes. Pairs of gametes then fuse to form zygotes, which give rise by meiosis to new sporozoites, and the cycle begins again. Apicomplexans are transmitted to new hosts in various ways; some, like the malaria parasite, are transmitted by infected mosquitos, while others may be transmitted in the feces of an infected host, or when a predator eats infected prey.

The class Coccidia tend to infect vertebrates through mucosal surfaces, such as the gut. The family Eimeriidae, within the order Eimeriida, within the Coccidia, is characterized by a life cycle that requires only one host and a brief passage through the environment in the oocyst stage. The genus Eimeria is characterized by sporozoite invasion, merogony, gamogony and oocyst formation confined to a region of the intestine that is specific to each Eimeria species. The oocysts sporulate in the environment, are ingested, and the oocyst walls are broken by bile salts and trypsin in the duodenum. The sporozoites penetrate the epithelial cell walls where they protect themselves with a specialized membrane. Within this safe haven, they produce as many as 120,000 merozoites as a schizont. Upon maturation, these burst out and invade other cells. This process is repeated two or three times and is followed by a gamont stage in which sexual reproduction occurs and oocysts are formed. These are excreted with the feces to complete the cycle.

In accordance with the present invention, an Apicomplexan organism at any developmental stage, e.g., a merozoite, a gamont, a gamontocyst, a gamete, a sporozoite, or an oocyst, can be used to treat or prevent an infectious disease caused with infection with HIV, or to elicit an immune response. In a preferred embodiment, a sporozoite, a merozoite or an oocyst of an Apicomplexan organism is used to treat or preventan infectious disease caused by infection with HIV, or to elicit an immune response. More preferably, a sporozoite, a merozoite or an oocyst of an Eimeria species is used to treat or prevent an infectious disease caused by infection with HIV, or to elicit an immune response.

In another embodiment of the invention, a dead Apicomplexan organism containing ARP is administered to a subject for treatment or prevention of an infectious disease caused by infection with HIV. An Apicomplexan organism can be killed by any method known in the art, including but not limited to, chemical toxins and radiations.

In yet another embodiment, a protein fraction, preferably, a membrane preparation or a storage granule preparation of an Apicomplexan organism containing ARP is administered to a subject for treatment or prevention of an infectious disease caused by infection with HIV.

### 5.3 Derivatives and Analogs of ARP

The invention further provides derivatives (including but not limited to fragments), and analogs of ARPs. The production and use of derivatives and analogs related to ARP are within the scope of the present invention.

In a specific embodiment, the derivative or analog is functionally active, i.e., capable of exhibiting one or more functional activities associated with a full-length, wild-type ARP. Preferably, the derivative or analog is an activator of dendritic cells as assayed by the DC assay. Derivatives or analogs of ARP can be tested for the desired activity by procedures known in the art

In particular, ARP derivatives can be made by altering ARP sequences by substitutions, insertions or deletions that provide for functionally equivalent molecules. Preferably, such alteration of an ARP sequence is done in a variable region (i.e., that is not highly conserved among different ARP molecules, for example, see FIG. 12), e.g., an alteration is done within the first fifteen amino acids of an ARP. Due to the degeneracy of nucleotide coding sequences, other DNA sequences which encode substantially the same amino acid sequence as an ARP gene may be used in the practice of the present invention. These include, but are not limited to, nucleotide sequences comprising all or portions of ARP genes which are altered by the substitution of different codons that encode a functionally equivalent amino acid residue within the sequence, thus producing a silent change. Likewise, the ARP derivatives of the invention include, but are not limited to, those containing, as a primary amino acid sequence, all or part of the amino acid sequence of an ARP including altered sequences in which functionally equivalent amino acid residues are substituted for residues within the sequence resulting in a silent change (i.e., conservative substitutions). For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. ARP derivatives of the invention also include, but are not limited to, those containing, as a primary amino acid sequence, all or part of the amino acid sequence of an ARP including altered sequences in which amino acid residues are substituted for residues with similar chemical properties (i.e., conservative substitutions). In specific embodiments, 1, 2, 3, 4, or 5 amino acids are substituted. In a specific embodiment, SEQ ID NO:1 amino acid residue 181 His can be changed to Tyr, and the new protein still has the same activity as SEQ ID NO:1. In a specific embodiment, SEQ ID NO:1 amino acid residue 183 Ser can be changed to Ala, and the new protein still has the same activity as SEQ ID NO:1. In another specific embodiment, SEQ ID NO:1 amino acid residue 181 His can be changed to Tyr, and amino acid residue 183 Ser can be changed to Ala, the new protein still has the same activity as SEQ ID NO:1.

Derivatives or analogs of ARP include, but are not limited to, those peptides which are substantially homologous to ARP or fragments thereof, or whose encoding nucleic acid is capable of hybridizing to an ARP nucleic acid sequence.

The ARP derivatives and analogs of the invention can be produced by various methods known in the art. The manipulations which result in their production can occur at the gene or protein level. For example, the cloned ARP gene sequence can be modified by any of numerous strategies known in the art (Maniatis, T., Molecular Cloning, A Laboratory Manual, 2d ed., Cold Spring Harbor Laboratory, Cold Spring Harbor, N.Y., 1989). The sequence can be cleaved at appropriate sites with restriction endonuclease(s), followed by further enzymatic modification if desired, isolated, and ligated in vitro. In the production of the gene encoding a derivative or analog of ARP, care should be taken to ensure that the modified gene remains within the same translational reading frame as ARP, uninterrupted by translational stop signals, in the gene region where the desired ARP activity is encoded.

Additionally, the ARP-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., J. Biol. Chem 1978, 253:6551), use of TAB.RTM. linkers (Pharmacia), etc.

Manipulations of the ARP sequence may also be made at the protein level. Included within the scope of the invention are ARP fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH₂— groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH₄, acetylation, formylation, oxidation, reduction; metabolic synthesis in the presence of tunicamycin, etc.

In addition, analogs and derivatives of ARP can be chemically synthesized. For example, a peptide corresponding to a portion of an ARP which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the ARP sequence. Non-classical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the ARP derivative is a chimeric, or fusion, protein comprising an ARP or fragment thereof fused via a peptide bond at its amino- and/or carboxy-terminus to a non-ARP amino acid sequence. In a preferred embodiment, the non-ARP amino acid sequence is fused at the amino-terminus of an ARP or a fragment thereof. In another preferred embodiment, the non-ARP amino acid sequence is fused at a variable region of an ARP or a fragment thereof, e.g., the first 15 amino acids of an ARP gene. In one embodiment, such a chimeric protein is produced by recombinant expression of a nucleic acid encoding the protein (comprising an ARP-coding sequence joined in-frame to a non-ARP coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding a mature ARP with a heterologous signal sequence is expressed such that the chimeric protein is expressed and processed by the cell to the mature ARP protein. The primary sequence of ARP and non-ARP gene may also be used to predict tertiary structure of the molecules using computer simulation (Hopp and Woods, Proc. Natl. Acad. Sci. U.S.A. 1981, 78:3824-3828); the chimeric recombinant genes could be designed in light of correlations between tertiary structure and biological function. Likewise, chimeric genes comprising an essential portion of ARP molecule fused to a heterologous (non-ARP) protein-encoding sequence may be constructed. In a specific embodiment, such chimeric construction can be used to enhance one or more desired properties of an ARP, including but not limited to, ARP stability, solubility, or resistance to proteases. In yet another embodiment, chimeric construction can be used to identify or purify an ARP of the invention, such as a His-tag, a FLAG tag, a green fluorescence protein (GFP), β-galactosidase, a maltose binding protein (MalE), a cellulose binding protein (CenA) or a mannose protein, etc.

In another specific embodiment, the ARP derivative is a fragment of ARP of one specie of Apicomplexa comprising a region of homology with an ARP of another specie of Apicomplexa. As used herein, a region of a first protein shall be considered “homologous” to a second protein when the amino acid sequence of the region is at least 25%, at least 30%, at least 45%, at least 55%, at least 65%, or at least 75% either identical or involving conservative changes, when compared to any sequence in the second protein of an equal number of amino acids as the number contained in the region.

In a specific embodiment, the invention provides ARP derivatives and analogs, in particular ARP fragments and derivatives of such fragments that comprise one or more domains of the ARP.

In a specific embodiment, relating to an ARP of a species other than Eimeria, preferably other species in the Apicomplexa phylum, the fragments comprising specific portions of ARP are those comprising portions in the respective ARP most homologous to specific fragments of an Eimeria ARP.

### 5.4 Antibodies to ARPs, Derivatives and Analogs

In various embodiments, monoclonal or polyclonal antibodies specific to ARP, or a domain of ARP, can be used in immunoassays to measure the amount of ARP or used in immunoaffinity purification of an ARP. A Hopp & Woods hydrophilic analysis (see Hopp & Woods, Proc. Natl. Acad. Sci. U.S.A. 1981, 78:3824-3828) can be used to identify hydrophilic regions of a protein, and to identify potential epitopes of an ARP. In a specific embodiment, an antibody that binds to SEQ ID NO:26 (bovine ARP fragment 1 (SEQ ID NO:3) fused to fragment 2 (SEQ ID NO:4) fused to fragment 3 (SEQ ID NO:5) fused to fragment 4 (SEQ ID NO:6) and fused to fragment 5 (SEQ ID NO:7)) is used in immunoassays to measure the amount of an ARP or in immunoaffinity purification of an ARP.

The antibodies that immunospecifically bind to an ARP or an antigenic fragment thereof can be produced by any method known in the art for the synthesis of antibodies, in particular, by chemical synthesis or preferably, by recombinant expression techniques.

Polyclonal antibodies immunospecific for ARP or an antigenic fragment thereof can be produced by various procedures well-known in the art. For example, an ARP can be administered to various host animals including, but not limited to, rabbits, mice, and rats, to induce the production of sera containing polyclonal antibodies specific for the ARP. Various adjuvants may be used to increase the immunological response, depending on the host species, including but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T Cell Hybridomas 563 681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding V_{H} and V_{L} domains are amplified from animal cDNA libraries (e.g., human or murine cDNA libraries of affected tissues). The DNA encoding the V_{H} and V_{L} domains are recombined together with an scFv linker by PCR and cloned into a phagemid vector. The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., J. Immunol. Methods 1995, 182:41-50; Ames et al., J. Immunol. Methods 1995, 184:177-186; Kettleborough et al., Eur. J. Immunol. 1994, 24:952-958; Persic et al., Gene 1997, 187:9-18; Burton et al., Advances in Immunology 1994, 57:191-280; International application No. PCT/GB91/O1 134; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., BioTechniques 1992, 12(6):864-869; Sawai et al., AJRI 1995, 34:26-34; and Better et al., Science 1988, 240:1041-1043.

To generate whole antibodies, PCR primers including V_{H} or V_{L} nucleotide sequences, a restriction site, and a flanking sequence to protect the restriction site can be used to amplify the V_{H} or V_{L} sequences in scFv clones. Utilizing cloning techniques known to those of skill in the art, the PCR amplified V_{H} domains can be cloned into vectors expressing a V_{H} constant region, e.g., the human gamma 4 constant region, and the PCR amplified V_{L} domains can be cloned into vectors expressing a V_{L} constant region, e.g., human kappa or lamba constant regions. Preferably, the vectors for expressing the V_{H} or V_{L} domains comprise an EF-1α promoter, a secretion signal, a cloning site for the variable domain, constant domains, and a selection marker such as neomycin. The V_{H} and V_{L} domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741.

A chimeric antibody is a molecule in which different portions of the antibody are derived from different immunoglobulin molecules. Methods for producing chimeric antibodies are known in the art. See e.g., Morrison, Science 1985, 229:1202; Oi et al., BioTechniques 1986, 4:214; Gillies et al., J. Immunol. Methods 1989, 125:191-202; and U.S. Pat. Nos. 5,807,715; 4,816,567; 4,816,397; and 6,311,415.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibody can be produced using variety of techniques known in the art, including but not limited to, CDR-grafting (European Patent No. EP 239,400; International Publication No. WO 91/09967; and U.S. Pat. Nos. 5,225,539, 5,530,101; and 5,585,089), veneering or resurfacing (European Patent Nos. EP 592,106 and EP 519,596; Padlan, Molecular Immunology 1991, 28(4/5):489-498; Studnicka et al., Protein Engineering 1994, 7(6):805-814; and Roguska et al., Proc. Natl. Acad. Sci. USA 1994, 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 2002, 169:1119-25, Caldas et al., Protein Eng. 2001, 13(5):353-60, Morea et al., Methods 2000, 20(3):267-79, Baca et al., J. Biol. Chem. 1999, 272(16):10678-84 (1997), Roguska et al., Protein Eng. 1996, 9(10):895-904, Couto et al., Cancer Res. 1995, 55 (23 Supp):5973s-5977s, Couto et al., Cancer Res. 1995, 55(8):1717-22, Sandhu JS, Gene 1994, 150(2):409-10, and Pedersen et al., J. Mol. Biol. 1994, 235(3):959-73. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., Nature 1988, 332:323.)

Further, the antibodies that immunospecifically bind to ARP or an antigenic fragment thereof can, in turn, be utilized to generate anti-idiotype antibodies that “mimic” ARP or an antigenic peptide thereof using techniques well-known to those skilled in the art. (See, e.g., Greenspan & Bona, FASEB J. 1989, 7(5):437-444; and Nissinoff, 1991, J. Immunol. 1991, 147(8):2429-2438).

### 5.4.1 Polynucleotide Sequences Encoding an Antibody

The invention provides polynucleotides comprising a nucleotide sequence encoding an antibody or fragment thereof that immunospecifically binds to ARP or an antigenic fragment thereof. The invention also encompasses polynucleotides that hybridize under high stringency, intermediate, or lower stringency hybridization conditions to polynucleotides that encode an antibody of the invention.

The polynucleotides may be obtained, and the nucleotide sequence of the polynucleotides determined, by any method known in the art. The nucleotide sequence of antibodies immunospecific for a desired antigen can be obtained, e.g., from the literature or a database such as GenBank. Once the amino acid sequences of an ARP or an antigenic fragment thereof is known, nucleotide sequences encoding this antibody or a fragment thereof (e.g., a CDR) can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., BioTechniques 1994, 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., J. Mol. Biol. 1998, 278: 457-479 for a listing of human framework regions). Preferably, the polynucleotide generated by the combination of the framework regions and CDRs encodes an antibody that specifically binds to a particular antigen. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

### 5.4.2 Recombinant Expression of an Antibody

Recombinant expression of an antibody of the invention, derivative, analog or fragment thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well-known in the art. See, e.g., U.S. Pat. No. 6,331,415. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication No. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715, and those described in Section 5.1.2, supra). The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 1983, 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 1986, 322:52; and Kohler, Proc. Natl. Acad. Sci. USA 1980, 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### 5.5 Structure Prediction and Functional Analysis of ARPs

The invention provides ARPs which can be from various species of Apicomplexa. Thus, it would be valuable if the structure of an ARP or a fragment thereof may be predicted based on the amino acid sequence. Structure prediction, analysis of crystallographic data, sequence alignment, as well as homology modeling, can be accomplished using computer software programs available in the art, such as BLAST, CHARMm release 21.2 for the Convex, and QUANTA v.3.3, (Molecular Simulations, Inc., York, United Kingdom). For example, some ARPs comprise a conserved PROF (smart 00392 or cd 00148) domain in Conserved Domain Database (CDD v. 1.60) as determined by a RPS-BLAST (Reverse Position-Specific BLAST) algorithm set to default parameters.

Once an ARP is identified, it may be isolated and purified by standard methods including chromatography (e.g., ion exchange, affinity, and sizing column chromatography), centrifugation, differential solubility, or by any other standard technique for the purification of proteins. The in vitro activities and in vivo biological functions of the foregoing may be evaluated using any suitable assay (including immunoassays as described infra).

Alternatively, once an ARP produced by a recombinant host cell is identified, the amino acid sequence of the ARP(s) can be determined by standard techniques for protein sequencing, e.g., with an automated amino acid sequencer.

The ARP sequence can be characterized by a hydrophilicity analysis (Hopp, T. and Woods, K., Proc. Natl. Acad. Sci. U.S.A. 1981, 78:3824). A hydrophilicity profile can be used to identify the hydrophobic and hydrophilic regions of the ARP and the corresponding regions of the gene sequence which encode such regions.

Secondary structural analysis (Chou and Fasman Biochemistry 1974, 13:222) can also be done to identify regions of the ARP that assume specific secondary structures.

Other methods of structural analysis can also be employed. These include, but are not limited to, X-ray crystallography (Engstom, A., Biochem. Exp. Biol. 1974, 11:7-13) and computer modeling (Fletterick, R. and Zoller, M. (eds.), Computer Graphics and Molecular Modeling, in Current Communications in Molecular Biology, Cold Spring Harbor Laboratory, Cold Spring Harbor, N.Y., 1986).

The functional activity of an ARP or a fragment thereof can be assayed by various methods known in the art.

For example, where one is assaying for the ability of an ARP from one genus to bind or compete with an ARP from another genus for binding to an antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipiting reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. Antibody binding can be detected by detecting a label on the primary antibody. Alternatively, the primary antibody is detected by detecting binding of a secondary antibody or reagent to the primary antibody, particularly where the secondary antibody is labeled. Many means are known in the art for detecting binding in an immunoassay and are within the scope of the present invention.

The half life of a protein is a measurement of protein stability and indicates the time necessary for a one half reduction in activity of the protein. The half-life of an ARP can be determined by any method measuring activity of ARP in samples from a subject over a period of time, such as but not limited to, DC assay and NK assay. The normalization to concentration of ARP in the sample can be done by immunoassays using anti-ARP antibodies (or antibody to a fused part of a gene wherein an ARP is fused to another moiety) to measure the levels of the ARP molecules in samples taken over a period of time after administration of the ARP, or detection of radiolabelled ARP molecules in samples taken from a subject after administration of the radiolabeled ARP molecules. In specific embodiments, techniques known in the art can be used to prolong the half life of an ARP in vivo. For example, albumin or inert polymer molecules such as high molecular weight polyethyleneglycol (PEG) can be used. See, e.g., International Publication Nos. WO 93/15199, WO 93/15200, and WO 01/77137; and U.S. Pat. No. 6,528,485.

Other methods will be known to the skilled artisan and are within the scope of the invention.

### 5.6 Characterization and Demonstration of ARP Activity

The immunostimulatory activity and/or anti-HIV activity of the ARPs used in accordance with the present invention can be determined by any method known in the art.

Various in vitro and in vivo assays that test the immunostimulatory activities and/or anti-HIV activities of an ARP are used in purification processes of an ARP. The protocols and compositions of the invention are also preferably tested in vitro, and then in vivo, for the desired therapeutic or prophylactic activity, prior to use in humans.

In one embodiment, a NK cell assay is used to assess ARP-induced cytotoxic activity of NK-like cells. Briefly, by way of example but not limitation, a NK cell assay can be carried out as follows: large granular lymphocytes (LGLs) are co-cultured with tumor cells at various ratios (effector to target ratio) for four days at 37° C., 5% CO₂. The culture medium also contains a low concentration of IL2 plus an ARP to be tested. The culture is terminated at the end of day 4 and vitally stained using a MTT cell viability quantification assay (see Mossman, T. J. Immunol. Methods 1983, 65:55; and Sigma Chemical MTT (M5655) product application note). Culture media is aspirated and the remaining cells are washed and replaced with DMEM/F12 plus FCS, containing MTT. The cells are further incubated for 4-5 hours at 37° C. Absorbance is measured with the aid of a plate reader. Decreased absorbance indicated a decrease in the number of viable cells per well (i.e., cytotoxicity). Absorbance is measured again after the MTT is solubilized by replacement of the medium with 2-propanol containing HCl, which gives a uniform color throughout the well. NK-inducing activity is calculated relative to negative (PBS/BSA) and positive (internal standard) controls.

In another embodiment, the ARP activity is measured by IFNY assay. By way of example but not limitation, the production and release of IFNγ by LGLs into the culture media is determined as follows: LGLs, enriched with NK cells, are isolated and seeded into 96 well plates at densities of 2.5 or 5.0×10⁵ cells/well, in 200 μL of DMEM/F12 supplemented with 10% fetal calf serium, gentamycin (50 μg/mL), and IL2 (125 U/mL). The ARP to be tested is added and the cells are cultured overnight, following which the condition media (CM) is removed and then centrifuged to remove any aspirated cells. Aliquots of the CM are then measured for IFNγ using an commercially available ELISA kit.

In a preferred embodiment, dendritic cell (DC) assay is used to follow interleukin-12 (IL-12) release from freshly isolated dendritic cells as an index of DC activation (see section 13). This activity is highly correlated with both NK-CMC in vitro and anti-tumor activity in vivo of an ARP. Briefly, by way of example but not limitation, a DC assay can be carried out as follows: freshly isolated dendritic cells (CD11c⁺ splenocytes, e.g., isolated from the spleens of BALB/c mice within ten hours of assaying the activity) are suspended at a density of 0.5×10⁶ cells per mL in cytokine supplemented culture media (GM-CSF, 1.0 ng/ml; IL-4, 1.0 ng/ml; IFNg, 3 ng/ml; anti mouse CD40; 0.5 mg/ml). The dendritic cells are then added (100 μL/well) to 96-well tissue culture plates containing ARP diluted in 100 μL media per well and cultured overnight at 37° C. in 5% CO₂, 95% air. Mouse IL-12 release from the dendritic cells (CD11c⁺ splenocytes) is then measured using ELISA.

Compounds for use in therapy can be tested in suitable animal model systems prior to testing in humans, including but not limited to in rats, mice, chicken, cows, monkeys, rabbits, etc.

Further, any assays known to those skilled in the art can be used to evaluate the prophylactic and/or therapeutic utility of the combinatorial therapies disclosed herein for treatment or prevention of an infectious disease caused by infection with HIV.

### 5.7 Uses of the Compositions of the Invention

The compositions of the invention may be administered to a subject in need thereof to stimulate an immune response, to prevent or treat an infectious disease caused by infection with HIV or to use as an alternative to interleukin 12 (IL-12) treatment. The compositions of the invention may be administered alone or in combination with one or more other therapies useful for immune stimulation to a subject in need thereof to elicit an immune response. The compositions of the invention may administered alone or in combination with one or more other therapies to a subject in need thereof to elevate systemic levels of IL-12 in the subject. The compositions of the invention may also be administered alone or in combination with one or more other therapies useful for the prevention or treatment of an infectious disease caused by infection with HIV.

In one embodiment, the present invention provides a method of treating or preventing an infectious disease caused by infection with HIV comprising administering to a subject in whom such treatment or prevention is desired a therapeutically or prophylactically effective amount of an ARP. In another embodiment, the present invention provides a method of treating or preventing an infectious disease caused by infection with HIV comprising administering to a subject in a whom such treatment or prevention is desired a therapeutically or prophylactically effective amount of a nucleic acid comprising a nucleotide sequence encoding one or more ARPs. In another embodiment, the present invention provides a method of treating or preventing an infectious disease caused by infection with HIV comprising administering to a subject in whom such treatment or prevention is desired a therapeutically or prophylactically effective amount of a cell transformed with a nucleic acid comprising a nucleotide sequence encoding an ARP, wherein said nucleotide sequence is operably linked to a promoter. In yet another embodiment, the present invention provides a method of treating or preventing an infectious disease caused by infection with HIV comprising administering to a subject in whom such treatment or prevention is desired a therapeutically or prophylactically effective amount of an ARP and a therapeutically or prophylactically effective amount of one or more agents other than ARP.

In another embodiment, the present invention provides a method of stimulating an immune response comprising administering to a subject an effective amount of an ARP. In another embodiment, the present invention provides a method of stimulating an immune response comprising administering to a subject an effective amount of nucleic acid comprising a nucleotide sequence encoding one or more ARPs. In another embodiment, the present invention provides a method of stimulating an immune response comprising administering to a subject an effective amount of a cell transformed with a nucleic acid comprising a nucleotide sequence encoding an ARP, wherein said nucleotide sequence is operably linked to a promoter. In yet another embodiment, the present invention provides a method of stimulating an immune response comprising administering to a subject an effective amount of an ARP and an effective amount of one or more immune stimulatory agents other than ARP. In a specific embodiment, the subject is infected with a human immunodeficiency virus (HIV). In specific embodiments, the subject is an avian species. In other specific embodiments, the subject is not an avian species.

In yet another embodiment, the present invention provides a method of elevating systemic level of interleukin-12 (IL-12) in a subject comprising administering to the subject in whom such elevation of interleukin-12 level is desired an effective amount of an ARP. In another embodiment, the present invention provides a method of elevating systemic level of interleukin-12 (IL-12) in a subject comprising administering to the subject in whom such elevation of interleukin-12 level is desired an effective amount of nucleic acid comprising a nucleotide sequence encoding one or more ARPs. In another embodiment, the present invention provides a method of elevating systemic level of interleukin-12 (IL-12) in a subject comprising administering to the subject in whom such elevation of interleukin-12 level is desired an effective amount of a cell transformed with a nucleic acid comprising a nucleotide sequence encoding an ARP, wherein said nucleotide sequence is operably linked to a promoter. In yet another embodiment, the present invention provides a method of elevating systemic level of interleukin-12 (IL-12) in a subject comprising administering to the subject in whom such elevation of interleukin-12 level is desired an effective amount of an ARP and an effective amount of one or more immune stimulatory agents other than ARP. In a specific embodiment, the subject is infected with a human immunodeficiency virus (HIV).

Several adjuvants are used experimentally to enhance the immune response to administered antigens. Only aluminum hydroxide (alum), however, is approved by the FDA as an adjuvant for human use. This inert inorganic molecule has been shown to be safe, but is only a moderately good adjuvant. Other adjuvants (e.g. Bacille Calmette-Guerin (BCG), Freund's adjuvants, keyhole limpet hemocyanin (KLH), dinitrophenol, QS-21, CAP have been used in animal studies or are currently under investigation for humans. Unfortunately, they are either non-specific (CAP, QS-21, dinitrophenol) or toxic (BCG, KLH, Freund's adjuvant).

In a further embodiment of the invention, the very potent dendritic cell activation properties of ARP make it an ideal candidate to provide a superior adjuvant for vaccine production. Inorganic adjuvants are considered non-specific activators of the immune system. In contrast, ARP appears to be a specific activator of innate immunity. Thus, it activates a cascade of genetically preprogrammed events that upregulate the antigen presenting activity of dendritic cells while simultaneously generating the release of the cytokines associated with the activation of immediate cellular responses (e.g. NK cells) and, more importantly, the activation of adaptive immunity (both cellular and humoral). This latter function would facilitate the generation of memory T-cells (cellular response) and circulating antibodies (humoral response).

The subject as used herein refers to an animal, preferably a mammal. In some embodiments, the subject is a domestic animal, including but not limited to, chicken, pig, cow, horse and goat. In some embodiments, the subject is a pet, including but not limited to, cat, dog, and bird. In a preferred embodiment, the subject is a human.

### 5.7.1 Gene Therapy

In one embodiment, one or more nucleic acid molecules comprising sequences encoding one or more ARPs are administered to a subject to stimulate an immune response and/or to prevent or treat an infectious disease caused by infection with HIV by way of gene therapy. Gene therapy refers to therapy performed by the administration to a subject of an expressed or expressible nucleic acid.

Any of the methods for gene therapy available in the art can be used according to the present invention. Exemplary methods are described below.

For general reviews of the methods of gene therapy, see Goldspiel et al., Clinical Pharmacy 1993, 12:488-505; Wu and Wu, Biotherapy 1991, 3:87-95; Tolstoshev, Ann. Rev. Pharmacol. Toxicol. 1993, 32:573-596; Mulligan Science, 1993, 260:926-932; and Morgan and Anderson, Ann. Rev. Biochem. 1993, 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred embodiment, a composition of the invention comprises nucleotide sequences encoding one or more ARPs, said nucleic acid sequences being part of expression vectors that express ARPs in a suitable host. In particular, such nucleic acids have promoters, preferably heterologous (non-native) promoters, operably linked to the ARP coding region, said promoter being inducible or constitutive, and, optionally, tissue-specific. In another particular embodiment, nucleic acid molecules are used in which the ARP coding sequences and any other desired sequences are flanked by regions that promote homologous recombination at a desired site in the genome, thus providing for intrachromosomal expression of the ARP nucleic acids (Koller and Smithies, Proc. Natl. Acad. Sci. USA 1989, 86:8932-8935; Zijlstra et al., Nature 1989, 342:435-438).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 1987, 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g, PCT Publications WO 92/06180, WO 92/22635, WO92/20316, WO93/14188, WO 93/20221. Alternatively, the nucleic acid can be introduced intracellularly and incorporated within host cell DNA for expression, by homologous recombination (Koller and Smithies, Proc. Natl. Acad. Sci. USA 1989, 86:8932-8935; Zijlstra et al., Nature 1989, 342:435-438).

In one embodiment, viral vectors that contain nucleic acids encoding one or more ARPs are used in accordance with the invention (see Miller et al., Meth. Enzymol. 1993, 217:581-599). A retroviral vector, for example, can be used in gene therapy to deliver an ARP to a subject. These retroviral vectors have been modified to delete retroviral sequences that are not necessary for packaging of the viral genome and integration into host cell DNA. More detail about retroviral vectors can be found in Boesen et al., Biotherapy 1994, 6:291-302; Clowes et al., J. Clin. Invest. 1994, 93:644-651; Kiem et al., Blood 1994, 83:1467-1473; Salmons and Gunzberg, Human Gene Therapy 1993, 4:129-141; and Grossman and Wilson, Curr. Opin. in Genetics and Devel. 1993, 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 1993, 3:499-503) present a review of adenovirus-based gene therapy. Bout et al., Human Gene Therapy 1994, 5:3-10) demonstrated the use of adenovirus vectors to transfer genes to the respiratory epithelia of rhesus monkeys. Other instances of the use of adenoviruses in gene therapy can be found in Rosenfeld et al., Science 1991, 252:431-434; Rosenfeld et al., Cell 1992, 68:143-155; Mastrangeli et al., J. Clin. Invest. 1993, 91:225-234; PCT Publication WO94/12649; and Wang et al., Gene Therapy 1995, 2:775-783. In a preferred embodiment, adenovirus vectors are used in gene therapy to deliver ARPs to a subject to prevent or treat an infectious disease caused by an infection with HIV.

Adeno-associated virus (AAV) has also been proposed for use in gene therapy (Walsh et al., Proc. Soc. Exp. Biol. Med. 1993, 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 1993, 217:599-618; Cohen et al., Meth. Enzymol. 1993, 217:618-644; Cline, Pharmac. Ther. 1985, 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include, but are not limited to, epithelial cells, endothelial cells, keratinocytes, fibroblasts, muscle cells, hepatocytes; blood cells such as T lymphocytes, B lymphocytes, NK cells, dendritic cells, monocytes, macrophages, neutrophils, eosinophils, megakaryocytes, granulocytes, autologous cancer cells, various stem or progenitor cells, in particular hematopoietic stem or progenitor cells, e.g, as obtained from bone marrow, umbilical cord blood, peripheral blood, fetal liver, etc.

In a preferred embodiment, the cell used for gene therapy is autologous to the patient.

In one embodiment in which recombinant cells are used in gene therapy, one or more nucleotide sequences encoding one or more ARPs are introduced into the cells such that the nucleotide sequences are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598, Stemple and Anderson, Cell 1992, 71:973-985; Rheinwald, Meth. Cell Bio. 1980, 21A:229; and Pittelkow and Scott, Mayo Clinic Proc. 1986, 61:771). In another specific embodiment, transformed cells are used.

In a specific embodiment, the nucleic acid to be introduced for purposes of gene therapy comprises a constitutive, tissue-specific, or inducible promoter operably linked to the coding region. In a preferred embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.8 Other Anti-Infection Agents

The present invention provides methods of preventing or treating an infectious disease caused by infection with HIV, said methods comprising administering to a subject in need thereof a composition comprising ARP alone or in combination with one or more prophylactic or therapeutic agents other than ARP, which treat opportunistic infections that accompany AIDS, or have anti-HIV activity. Any agent or therapy which is known to be useful, or which has been used or is currently being used for the prevention or treatment of opportunisstic infectious disease or HIV infection can be used in combination with the composition of the invention in accordance with the invention described herein.

Examples of such agents to treat opportunistic bacteria infections include, but are not limited to, folate antagonists (e.g., mafenide, silver sulfadiazine, succinylsulfathiazole, sulfacetamide, sulfadiazine, sulfamethoxazole, sulfasalazine, sulfisoxazole, pyrimethoamine, trimethoprim, co-trimoxazole), inhibitors of cell wall synthesis (e.g., penicillins, cephalosporins, carbapenems, monobactams, vacomycin, bacitracin, clavulanic acid, sulbactam, tazobactam), protein synthesis inhibitors (e.g., tetracyclines, aminoglycosides, macrolides, chloramphenicol, clindamycin), fluoroquinolones (e.g., ciproloxacin, enoxacin, lomefloxacin, norfloxacin, ofloxacin), nalidixic acid, methenamine, nitrofurantoin, aminosalicylic acid, cycloserine, ethambutol, ethionamide, isoniazid, pyrazinamide, rifampin, clofazimine, and dapsone.

Examples of agents that can be used in combination with ARP to treat opportunistic fungal infections include, but are not limited to, amphotericin B, fluconazole, flucytosine, itraconazole, ketoconazole, clotrimazole, econazole, griseofulvin, miconazole, and nystatin.

Examples of agents that can be used in combination with ARP to treat opportunistic protozoal infections include, but are not limited to, chloroquine, dehydroemetine, diloxamide furoate, emetine, metronidazole, paramomycin, chloroquine, mefloquine, primaquine, pyrimethamine, quinine, quinidine, melarsoprol, nifurtimox, pentamidine, suramin, sodium stibogluconate, pyrimethamine, and quinacrine.

Examples of antihelmintic agents that can be used in combination with ARP to treat opportunistic helminth (worm) infections include, but are not limited to, ivermectin, mebendazole, pyrantel pamoate, thiabendazole, praziquantel, and niclosamide.

Examples of antiviral agents that can be used in combination with ARP to treat HIV infection include, but are not limited to, amantadine, ribavirin, rimantadine, acyclovir, famciclovir, foscarnet, ganciclovir, trifluridine, vidarabine, didanosine (ddI), stavudine (d4T), zalcitabine (ddC), zidovudine (AZT), lamivudine, abacavir, delavirdine, nevirapine, efavirenz, saquinavir, ritonavir, indinavir, nelfinavir, amprenavir, lopinavir and interferon.

### 5.9 Dosage Regimens

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

The dosage of ARP for intravenous administration in a human patient provided by the present invention is preferably less than 100 μg/kg body weight, less than 50 μg/kg body weight, less than 10 μg/kg body weight, less than 5 μg/kg body weight, less than 1 μg/kg body weight, less than 0.5 μg/kg body weight, less than 0.1 μg/kg body weight, less than 0.05 μg/kg body weight, less than 0.01 μg/kg body weight, or less than 0.001 μg/kg body weight. In a preferred embodiment, ARP is given at a dosage of 0.1 μg per person per day (or about 0.14 ng per kg body weight).

Prior to administering the first full dose, each patient preferably receives a subcutaneous injection of a small amount (e.g., 1/100 to 1/10 of the prescribed dose) of a composition of the invention to detect any acute intolerance. The injection site is examined one and two hours after the test. If no reaction is detected, then the full dose is administered.

The compositions of the invention can also be administered orally. In one embodiment, intact sporulated oocysts of an Apicomplexan genus (e.g., Eimeria tenella) are given orally with drinking water. The dosage can be, by way of example, 100 to 10,000 oocysts in a single administration depending on the cross-species infectivity of the protozoan.

The compositions of the invention can be administered in various forms. In one embodiment, a composition of the invention comprises an ARP in a membrane-linked form. In another embodiment, a composition of the invention comprises live or dead bacteria with recombinant ARP expressed. In another embodiment, a composition of the invention comprises live bacteria, which are specific to a particular organ, expressing recombinant ARP (e.g., Lactobacillus would live in intestinal tissue, while some air-born non-pathogenic bacteria may be specific to lungs, or the nasal cavity, etc).

### 5.10 Administrations, Formulations and Kits

The invention provides methods of treatment and/or prophylaxis by administration to a subject of an effective amount of a composition comprising ARP. In a preferred embodiment, the ARP is purified. The subject is preferably an animal, including but not limited to animals such as cows, pigs, chickens, etc., and is preferably a mammal. In a preferred embodiment, the subject is a domestic animal, including but not limited to chickens, pigs, cows, goats and horses. In another preferred embodiment, the subject is a pet, including but not limited to, cats, dogs, and birds. In another preferred embodiment, the subject is a human. The phrase “therapeutically or prophylactically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically or prophylatically effective amount is sufficient to cause an improvement in a clinically significant condition in the host.

Various delivery systems are known and can be used to administer a composition of the invention, e.g., encapsulation in liposomes, microparticles, microcapsules, expression by recombinant cells, receptor-mediated endocytosis (see, e.g., Wu and Wu, J. Biol. Chem. 1987, 262:4429-4432), construction of an ARP nucleic acid as part of a retroviral or other vector, etc. Methods of introduction include, but are not limited to, intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved by, for example, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, a chimeric construction of an ARP is used to target a specific area, i.e., even systemic administration of the composition would direct the composition to the organ of choice.

In a specific embodiment, the ARP nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 1991, 88:1864-1868), etc. Alternatively, an ARP nucleic acid can be introduced intracellularly and incorporated within host cell DNA for expression, by homologous recombination.

The present invention also provides pharmaceutical compositions. Such compositions comprise a therapeutically or prophylatically effective amount of a composition of the invention, and a pharmaceutically acceptable carrier or excipient. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin. Such a carrier includes but is not limited to saline, buffered saline, dextrose, water, glycerol, ethanol, and combinations thereof. The carrier and composition can be sterile. The formulation should suit the mode of administration. The phrase “pharmaceutically effective amount” refers to therapeutically or prophlyactically effective amounts of a composition of the invention administered to a subject in pharmaceutically acceptable formulations or dosage forms.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In one embodiment, purified Apicomplexan organisms are given orally. In a specific embodiment, intact sporulated oocysts of an Apicomplexa genus (e.g., Eimeria species, such as E. tenella) are given orally with drinking water.

In addition to the formulations described previously, the compositions may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the composition of the invention in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention sufficient for a single administration.

In a specific embodiment, a kit comprises a first container containing a composition comprising ARP; and a second container containing a different treatment modality in an amount that, when administered before, concurrently with, or after the administration of the ARP in the first container, is effective to improve overall treatment effectiveness over the effectiveness of the administration of each component alone, or is effective to decrease side effects of the treatment when each component is used alone. In a preferred specific embodiment, the invention provides a kit comprising in a first container, a composition of the invention; and in a second container, a composition comprising a purified cytokine. The appropriate and recommended dosages, formulation and routes of administration for treatment modalities such as chemotherapeutic agents, radiation therapy and biological/immunotherapeutic agents such as cytokines are known in the art and described in such literature as the Physician's Desk Reference (56th ed., 2002).

### 5.11 Article of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular or subcutaneous delivery. Thus, the invention encompasses solutions, preferably sterile, suitable for each delivery route.

In another preferred embodiment, compositions of the invention are stored in containers with biocompatible detergents, including but not limited to, lecithin, taurocholic acid, and cholesterol; or with other proteins, including but not limited to, gamma globulins and serum albumins. More preferably, compositions of the invention are stored with human serum albumins for human uses, and stored with bovine serum albumins for veterinary uses. While not bound by any theory, a low concentration of ARP tends to be denaturing on the surface of plastic or glass containers. The handling and storage as described herein would minimize the loss of ARP on surfaces of containers and keep an ARP in its active form. Other methods known in the art can also be used. See Wang, International J. of Pharmaceutics, 1999, 185:129-188.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question, e.g., as described herein. In other words, the article of manufacture includes instruction means indicating or suggesting a dosing regimen including, but not limited to, actual doses, monitoring procedures and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising a needle or syringe, preferably packaged in sterile form, for injection of the formulation, and/or a packaged alcohol pad.

In a specific embodiment, an article of manufacture comprises packaging material and a pharmaceutical agent and instructions contained within said packaging material, wherein said pharmaceutical agent is an ARP or a derivative, fragment, homolog, analog thereof and a pharmaceutically acceptable carrier, and said instructions indicate a dosing regimen for preventing, treating or managing a subject with HIV. In another embodiment, an article of manufacture comprises packaging material and a pharmaceutical agent and instructions contained within said packaging material, wherein said pharmaceutical agent is an ARP or a derivative, fragment, homolog, analog thereof, a prophylactic or therapeutic agent other than an ARP or a derivative, fragment, homolog, analog thereof, and a pharmaceutically acceptable carrier, and said instructions indicate a dosing regimen for preventing, treating or managing a subject with HIV. In another embodiment, an article of manufacture comprises packaging material and two pharmaceutical agents and instructions contained within said packaging material, wherein said first pharmaceutical agent is an ARP or a derivative, fragment, homolog, analog thereof and a pharmaceutically acceptable carrier, and said second pharmaceutical agent is a prophylactic or therapeutic agent other than an ARP or a derivative, fragment, homolog, analog thereof, and said instructions indicate a dosing regimen for preventing, treating or managing a subject with a HIV.

The present invention may be better understood by reference to the Examples, which are provided for purposes of illustration of the invention and not by way of limitation.

### 6. EXAMPLE 1

### Purification of ARP from Bovine Small Intestine Extracts

### 6.1 Material

Source of intestines used in the examples: large animal intestines were obtained from freshly slaughtered cows. These were routinely obtained from Bellingar Packing, Ashley, Mich. Rat and mouse intestines were obtained from Sprague Dawley rats or BALB-c mice, purchased from Harlan Industries, Indianapolis, Ind. Pig intestines were obtained from Bellingar Packing. Some pig and cow intestines were also obtained from Bain's Packing & Refrigeration, Howell, Mich.

### 6.2 Methods

### 6.2.1 Enriched or Isolated ARP

Extraction and Centrifugation

Bovine small intestine ileal sections (25 feet or 40 feet) proximal to the cecum were excised from animals immediately after slaughter. Each section was slit down its length and the contents flushed free. Washing was initiated at the slaughterhouse in phosphate buffered saline (PBS) plus ciprofloxacin (10 mg/L) and was completed in the laboratory. The tissue was transported on ice to the laboratory where it was cut into 1 foot long strips and the external fatty deposits removed. Each section was washed in a solution of PBS containing ciprofloxacin. This was followed by serial washes through 6 beakers (on ice) containing PBS without ciprofloxicin. Each section was blotted free to remove excess fluid and then the soft tissue was scraped from the serosal casing and the serosa was discarded. The soft tissue was minced in a kitchen-variety motorized mini food processor in 50 mL aliquots using 10 start and stop cycles of the processor. An equal volume of cold 30 mM Hepes was added and the mixture was homogenized in a 55 mL glass homogenizer tube with a Teflon pestle. After five down-up cycles in the homogenizer, the material was centrifuged at 18,000 rpm (25,000 G) in a refrigerated SS-34 Sorvol rotor. The resulting liquid supernatants were prefiltered through a double layer of Millipore pre filters (#AP1507500) housed in a sterilizing stainless 90 mm filter holder to remove fatty particulates in suspension. The filtrate was flash frozen in liquid nitrogen and stored frozen at −80° C.

Pretesting of Prefiltered BEX

Preparation:

(a) Prepare 2.0 L of 1×PBS buffer from 10×PBS and acclimate to 4° C.

(b) Soak an appropriate number (one greater than the number of intestines to be pre-tested) of 100 mm long, 10 mm wide regenerated cellulose dialysis membranes, MWCO 6-8000, in r.o.deionized water until pliable.

(c) Defrost the 1.2 ml/intestine aliquots of prefiltered BBX and the intestinal mix.

Test for Activity:

(a) Dialyze 1.0 ml of the individual intestinal extracts and intestinal mix against 1 L of PBS for 3 hours at 4° C.

(b) Change buffer and dialyze overnight.

(c) Transfer dialysates to sterile 1.2 ml cryotubes.

(d) Prepare individual samples for the dendritic cell activity test by diluting the dialyzed stock BBX 1:10 (1 part BBX plus 9 parts sterile 0.1% BSA in PBS). From each stock serially dilute 1:2 (50 μl stock plus 50 μl 0.1% BSA/PBS) 4×.

(e) Graphically compare pre-test samples with an appropriate pre-test control with predetermined activity.

(f) Flash freeze and store pre-test leftovers in the −80° C. freezer.

Process Control:

Discard extracts from intestines generating dendritic cell activity less than 20% of the pre-test control.

Procipitate™ and Cleanascite™

The initial reductions of non-active macromolecules were facilitated with two commercial bulk-processing products from Ligochem, Inc. (Cleanascite™ and Procipitate™). Three parts extract was treated with one part Cleanascite™, which is a non-ionic adsorbent which selectively removes lipids, floating cells, cell debris and mucinous impurities. After centrifugation, the resulting supernatant was treated with the synthetic, water insoluble, anionic polyelectrolyte Procipitate™ at a ratio of four parts supernatant to one part Procipitate™ to remove selective proteins. This was spun at low speeds according the manufacturers recommendation and the supernatant retained for further purification.

Ultra-Filtration

A small sample of each week's batch was purified for testing using an Amicon stirred cell (series 800) initially fitted with a 300 kD disc membrane to remove impurities greater than 300 kD in size followed by a 10 kD disc membrane to remove impurities less than 10 kD as well as to concentrate the sample. During this procedure phosphate buffered saline was used to wash the >300 kD retentate. The resulting 10 to 300 kD sample was tested on the NK assay and/or in mice.

If the sample was active, the whole batch was processed in a large-scale procedure. The solution was filtered through 300 kD filter plates in a Millipore Pelicon™ ultrafiltration system and the <300 kD flow-through material together with its 30 mM HEPES wash(es) were concentrated in Amicon™ stirred cells on 10 kD disc membranes. The resulting material, denoted Stage 1 BEX (Bovine Extract), was sterile-filtered with Serum Acrodisc™ 0.22 μm (Gelman Cat# 4525) filters, flash frozen, and stored at −80° C. awaiting Stage 2 BEX purification.

Superdex™ Size Exclusion

Gel filtration chromatography was used to further purify Stage 1 into Stage 2. A 1200 ml column (Amicon Cat# 9644100) was packed with Superdex-75 prep grade (Pharmacia Cat# 17-1044-01) and equilibrated in 15 mM Sodium Phosphate, 120 mM Sodium Chloride pH 7.0 (PBS). PBS buffer was used for isocratic elution of fractions. Prior to loading, Stage 1 BEX was concentrated in CentriPrep-10™ (Amicon Cat# 4304) to 35-40 mg/ml, filtered using Serum Acrodisc™ 0.22 μm (Gelman Cat# 4525). Glycerol was added to a final concentration of 5%, and the sample was loaded onto the column at 0.1-1% of column volume (1.2-12 ml). Chromatographic parameters used: 280 nm, 1.0 Absorbance full scale, 4.0 ml/min (18-26 psi) flow rate, 5 cm/hr chart speed and 2.0 minutes/8.0 mL fractions size.

Active fractions were pooled and concentrated to ˜40 mg/ml using Amicon Stir Cell™ Model #8200 (Amicon Cat# 5123) with a YM10™ disc membrane (Amicon Cat# 13632). Stage 2 was sterile filtered and stored at −80° C. for the next purification step.

DEAE

A DEAE-Sepharose column (1.5×6 cm, DEAE-Sepharose Fast Flow, Pharmacia Biotech Inc. Piscataway, N.J.) was equilibrated with 30 mM Hepes and 0.1 M NaCl, pH 7.4. The sample obtained from Superdex 75™ (Stage 2) in PBS was applied to the column. The column was then washed with 100 ml of 0.1 M NaCl in 30 mM Hepes and then 200 ml of 0.2 M NaCl in the same buffer. The column was eluted isocratically with approximately 120 ml of 0.3 M NaCl in Hepes buffer. Fractions were collected every 4 min at 2.5 ml/fraction. The column was further eluted with 0.5 M NaCl in 30 mM Hepes. Fractions were analyzed by the NK-mediated cytotoxicity assay and SDS-polyacrylamide gel electrophoresis.

### 6.2.2 Purification of ARP: Modified Method

Extraction and Centrifugation

See the same step in section 6.2.1.

Pretesting of Prefiltered BEX

See the same step in Section 6.2.1.

Ammonium Sulfate Partitioning

The extract supernatant was brought to 45% ammonium sulfate at 4° C. using solid chemical and stirred for 1 hour. This was centrifuged in 250 mL centrifuge bottles at 13,000 RPM (17,300 G) for 30 minutes and in 40 mL centrifuge tubes at 15,000 RPM (17,500 G) for 20 min. The supernatant was decanted into a 4 L glass beaker and the pellets were discarded. The supernatants were brought to 80% saturation using solid ammonium sulfate, centrifuged as above, and the supernatant discarded. The pellets were dissolved in 1.5M Ammonium Sulfate/50 mM Na Phosphate (pH 6.8) after resuspending completely within the bottles or tubes. Non-dissolved material was removed by centrifugation in 250 mL centrifuge bottles at 10,000 RPM (10,200 G) for 30 minutes. The supernatants were pre-filtered through a double layer of Millipore pre-filters (AP1507500) housed in a sterilizing stainless 90 mm filter holder. Samples were taken after each step for quality control monitoring.

Hydrophobic Interaction Column (hereinafter “HIC”) Step-Elution Chromatography

A 5 cm×20 cm chromatography column, packed with 300 mL phenyl sepharose 6 fast flow (high substitution)—Amersham Pharmacia Biotech 17-0973-05, was equilibrated with 1.5M ammonium sulfate/50 mM Sodium Phosphate buffer at pH 6.8. At room temperature the pre-filtered ammonium sulfate treated BBX was pumped onto the column at a rate of ˜18 mL/min. Flow was collected from the column until no longer cloudy (˜1200 mL). The column was washed with 900 mL of 1.5M Ammonium Sulfate/50 mM Sodium Phosphate. The active material was eluted with 700 ml of 0.5 M Sodium Phosphate buffer.

The eluted material was prepared for DEAE chromatography by dialysis against 4 L of 0.2M NaCl/10 mM Na Phosphate (pH7.4) at 4° C. with buffer changes after 3 and 6 hours followed by a final dialysis overnight.

DEAE Step-Elution Chromatography

After the sample was dialyzed against 0.2 M NaCl, 10 mM sodium phosphate, pH 7.4, the sample was filtered through a 0.45 μm membrane to remove particulates, and then applied to the DEAE-Sepharose column (0.5×8 cm). The column was washed with about 800 mL of the same buffer (0.2 M NaCl, 10 mM Na Phosphate, pH 7.4). When the absorbance at 280 nm was down to baseline, the bound material was eluted with about 400 mL of the eluted buffer (0.5 M NaCl, 10 mM Na Phosphate, pH 7.4). The eluted fraction was concentrated to about 20 mL by Amicon stir cell with YM 10 membrane. The sample was divided into two halves and flash frozen in liquid nitrogen and then stored at −80° C.

Superdex™ Size Exclusion

Gel filtration chromatography was used to further purify the material from the DEAE step-elution. A 1200 mL column (Amicon Cat# 9644100) was packed with Superdex-75 prep grade (Pharmacia Cat# 17-1044-01) and equilibrated in 15 mM Sodium Phosphate, 120 mM Sodium Chloride pH 7.0 (PBS). PBS buffer was used for isocratic elution of fractions. Prior to loading, the samples was filtered using Serum Acrodisc™ 0.22 μm (Gelman Cat# 4525) and loaded onto the column at 0.1-1% of column volume (1.2-12 ml). Chromatographic parameters used: 280 nm, 1.0 Absorbance full scale, 4.0 ml/min (18-26 psi) flow rate, 5 cm/hr chart speed and 2.0 minutes/8.0 ml fractions size. The sample was flash frozen in liquid nitrogen and stored at −80° C.

Concentration of Superdex™ Samples and Calmodulin Removal

The active regions from 7 batches were thawed and pooled, which typically generated approximately 1,200 mL. The sample was centrifuged at 10,000 rpm (10,200 G) for 30 min with GSA rotor to remove any precipitate. The sample was then filtered with 0.45 μm syringe filter and applied to the DEAE-Sepharose column (0.3×5.5 cm). The column was washed with 0.2 M NaCl, 10 mM Na Phosphate, pH 7.4 (about 200 mL). The bound material was eluted with 1 M NaCl, 0.1 mM CaCl₂, 10 mM Tris, pH 7.0. The protein peak was collected in approximately 50-60 mL and was dialyzed overnight against 1 liter of 1 M NaCl, 0.1 mM CaCl₂, 10 mM Tris, pH 7.0.

To remove calmodulin and other calcium-binding proteins, an HIC column (1.5×6 cm) was equilibrated with 1 M NaCl, 0.1 mM CaCl₂, 10 mM Tris, pH 7.0 before the sample was loaded onto the column. After applying the sample, the column was washed with the same buffer. The flow through fraction was collected and constituted about 75 to 80 ml. The column was washed with the same buffer (about 100 mL) then the bound material was eluted with 1 mM EGTA (ethylene glycol-bis(p-aminoethyl ether)-N,N,N′,N′-tetraacetic acid), in 10 mM Tris, pH 7.0. The flow through fraction, which contained all DC activity, was used for the next step.

HIC Gradient Chromatography

All high performance liquid chromatography (hereinafter “HPLC”) was performed with a Waters Associates (Milford, Mass.) 626 LC system and a Linear Instruments (Reno, Nev.) 200 Uvis absorbance detector at either 280 nm (HIC) or 215 nm (DEAE and C-8 separations). All chemicals are Ultrapure Bioreagent grade from J. T. Baker (Phillipsburg, N.J.) unless otherwise noted.

Sample volume was measured and mixed with an equal volume of 3.0 M ammonium sulfate (ACS grade, Mallinckrodt, Paris Ky.) solution in 50 mM phosphate buffer, pH 7.2. The sample was loaded via the solvent inlet manifold on the HPLC. Solvents are: Buffer A, 1.5 M ammonium sulfate in 50 mM phosphate buffer, pH 7.2 and Buffer B, 50 mM phosphate buffer, pH 7.2. Samples were applied to 21.5×150 mm Phenyl-5PW column, (TosoHaas, Montgomeryville, Pa.) with 13 μm particle size, equilibrated in Buffer A at a flow rate of 4 mL/min at room temperature. At this time a linear gradient was performed from 0-100% Buffer B over 90 minutes at 4 mL/min. Fractions were collected at 2.5 minutes using a fraction collector (LKB-Frac-100, Pharmacia, Piscataway N.J.). Fractions were diluted with phosphate buffered saline (PBS) and assayed on the DC assay. Active fractions were pooled and dialyzed in Spectra/Por 1 Membrane dialysis tubing, 6-8000 MWCO (Fisher, Pittsburgh Pa.) against two changes of PBS.

DEAE Gradient Chromatography

Samples were loaded via the solvent inlet manifold on the HPLC. Solvents are: Buffer C, PBS, pH 7.2 and Buffer D, 0.5 M NaCl in 15 mM phosphate buffer, pH 7.2. Samples were applied to DEAE-5PW column, 10 μm bead, 8×75 mm (TosoHaas) equilibrated in Buffer C at 1 mL/min at room temperature. The column was flushed with Buffer C until a stable baseline was established. At this time a linear gradient was performed from 0-100% Buffer D over 90 minutes at 1 mL/min. Fractions were collected by hand to capture either individual peaks or at five-minute intervals using the fraction collector. Fractions were diluted with PBS and assayed on the DC assay.

C8 Reverse-Phase Chromatography

Samples were loaded either via the solvent inlet manifold for large volumes (>5 mL) or via the injector loop for smaller samples. Solvents are: Buffer E, 5% Acetonitrile (HPLC grade, Burdick and Jackson, Muskegon Mich.)/95% water (HPLC grade, Burdick and Jackson)/0.1% Trifluoroacetic acid (TFA); and Buffer F, 90% HPLC Acetonitrile/10% HPLC water/0.08% TFA. Samples were applied to C8, 5 μm bead, 300 Å, 4.6×150 mm column (Vydac 208TP5415, The Separations Group, Hesperia Calif.) equilibrated with Buffer E at 1 mL/min at 55° C. The column was flushed with Buffer E until a stable baseline was established. At this time, a linear gradient was performed from 30%-55% Buffer F in 150 minutes at 1 mL/min and 55° C. Fractions were collected either at five-minute intervals or for individual peaks. Fractions were diluted with PBS and assayed for activity on the DC assay. The bulk of the fractions were flash frozen in liquid nitrogen and stored at −80° C.

### 6.3 Results

Enriched or Isolated ARP:

Small intestine tissues were obtained approximately once per week, typically from six freshly slaughtered cattle. The extraction & centrifugation, Procipitate™ & Cleanascite™, and ultra-filtration steps produced a soluble mixture of macromolecules with the approximate size range of 10-300 kD. This material was non-toxic, could induce NK cytotoxicity in vitro, induce IFNγ in NK cells in vitro, and induce IL-12 release from dendritic cells in vitro. The solution was sampled for mycoplasma and bacterial testing (MSU Animal Health Diagnostic Laboratory) and measured for LAL reactivity. It was prepared for size exclusion (Stage 2) purification by concentration on ultrafiltration membranes. Separation on the Superdex™ molecular sieving column yielded activity confined to a single region, which is centered around 20-30 kD.

To enhance purity of Stage 2 (size exclusion purification) material and reduce any endotoxin burden, the concentrated active fractions of the 1st Superdex™ separation were applied to the column again. The central portion of the peak in the re-run was retained and denoted BBX-01 and used in the 1 st leg of the phase 1 clinical trial approved by the FDA. This material was sterile filtered and concentrated on ultrafiltration membranes prior to sterile filling in dosage forms at the GMP formulating facility at the University of Iowa.

DEAE ion exchange chromatography was used to purify Stage 2 material into Stage 3 material. The active portions of the re-run material from the Superdex™ separations were applied to a freshly cleaned DEAE Sepharose™ column. Due to the high ionic strength (0.15M) of the Stage 2 material, the majority of the protein passed through the column unbound. This is denoted Fraction A. The bound material was eluted in two steps of increasing ionic strength (0.2M and 0.5M). The first elution is denoted Fraction B and the second, Fraction C. Two types of activity have been detected in Fractions A and C, respectively, but Fraction B has routinely exhibited weak activity. The activity of Fraction A is associated with induction of high levels of IFNγ, which was proved to be IL18. Fraction C contains the majority of the NK-inducing activity and is the only fraction that activates dendritic cells. The C fraction contains only 5% of the protein and is denoted BBX-01c. This material was used for the 2nd leg of the human phase 1 clinical trial.

Purification of ARP: Modified Method

The modified purification procedures were developed to enhance recovery and specific activity. The modified procedure increased the yield of ARP at the final step possibly by as much as 10²-10³ fold. The first part of the procedure used an implementation of a long-standing method to process crude material utilizing ammonium sulfate fractionation. It was done in two steps. The first step removed approximately 50% of protein contaminants together with some other material, and inhibited proteolysis of ARP by internal proteases, while the second step precipitated ARP allowing to reduce the volume and speed up further purification steps, also minimizing proteolysis. Prevention of proteolysis was most probably responsible for the yield increase and for the increase in the average length of sequenced ARP peptides, which allowed identification of the Eimeria surface antigen by database searches. Experiments to find the best conditions yielded 45% saturation ammonium sulfate for the first precipitation, followed by 80% saturation for the second precipitation. The activity was recoverable in the final precipitation pellet, which was resuspended for further purification.

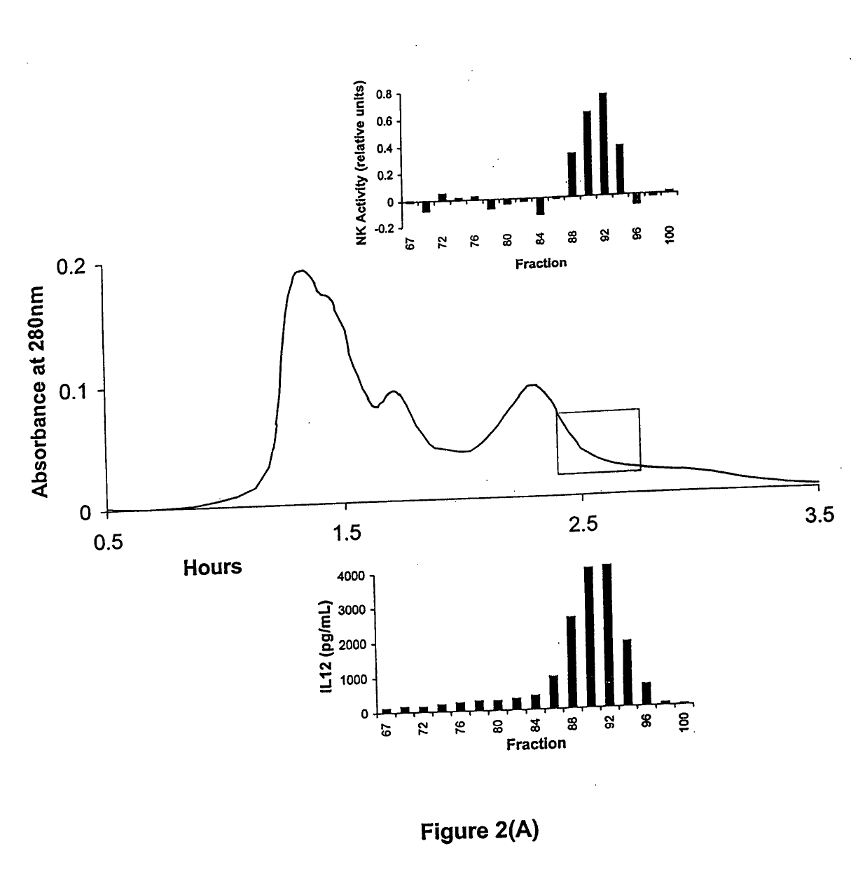

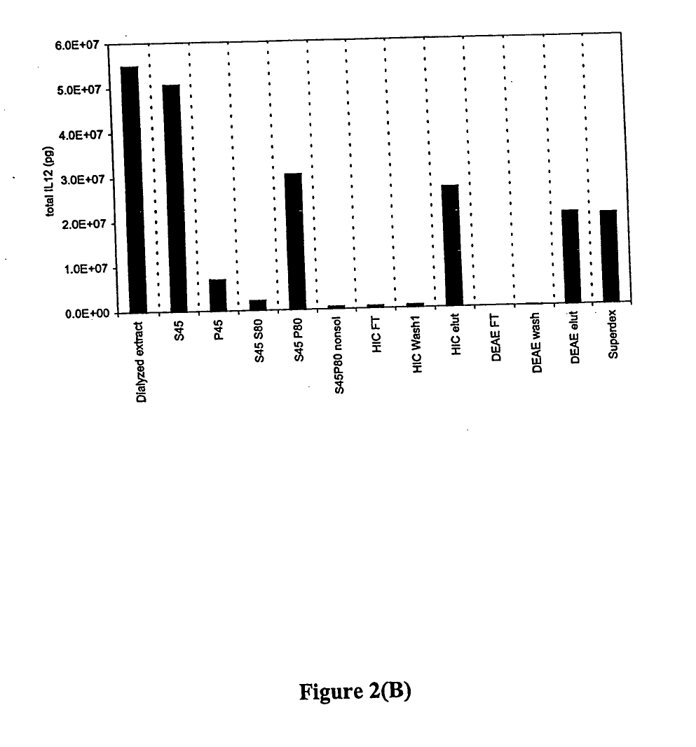

To reduce the macromolecular load prior to Superdex™ size-exclusion chromatography, two bulk processing steps were incorporated. The first step used an HIC column, which is compatible with the re-dissolved pellet of the ammonium sulfate partitioning process. A large amount of inactive material, particularly nucleic acid, was removed in the flow-through and wash. The active material was eluted after a change to a buffer free of ammonium sulfate. The elution was processed in a similar fashion on DEAE ion exchange chromatography where the flow-through and wash were discarded. The elution at higher NaCl concentration was retained, concentrated on a membrane pressure cell, and added to the Superdex™ column and the activity was eluted in a region of low absorbance (FIG. 2(A)). The total recovery of activity, for a typical batch (1 week) is shown in FIG. 2(B). Specific activity (with respect to protein content) was determined on the retained fractions from each step in the purification (Table 2). Approximately 7000-fold purification was reached at the end of the Superdex step.

TABLE 2
Purification steps that were performed on each batch

| Purification | Activity | Activity | Total | Specific |
|---|---|---|---|---|
| Step | Location | Recovery | Protein | Activity |
| Extract | Supernatant | 100 | 25504 | 1.0 |
| 45% Ammonium | Supernatant | 102 | 11684 | 2.2 |
| Sulfate cut | | | | |
| 80% Ammonium | Pellet | 80 | 11035 | 1.8 |
| Sulfate cut | | | | |
| Hydrophobic | PBS Elution | 59 | 6683 | 2.2 |
| Interaction | | | | |
| Step Elution | | | | |
| DEAE Step Elution | Salt Elution | 65 | 460 | 36.2 |
| Size Exclusion | Selected | 62 | 2.3 | 6820.0 |
| (Superdex ™) | Fraction | | | |
| Chromatography | | | | |

Since bacterial endotoxin was always a contaminant from small intestine extracts, the endotoxin levels were monitored with the LAL assay at each step in the purification. This is shown in FIG. 2(B) for the steps shown in FIG. 2(A). Endotoxin levels are reduced approximately 1000-fold resulting in final levels that are well below those that can interfere with the DC assay (threshold of 1000 U/mL for minimal DC activity).

To obtain enough material for final purification, pools were created that contained the extracts from 21 cattle intestines. These were pre-selected for high activity in the crude homogenate supernatants and processed three-at-a-time. Seven batches of 3 intestinal preparations were combined, concentrated, cleared of calcium binding proteins and designated as a pool. Four such pools were created. The first pool was used for method development and the second pool was used to obtain the first sequenceable product.

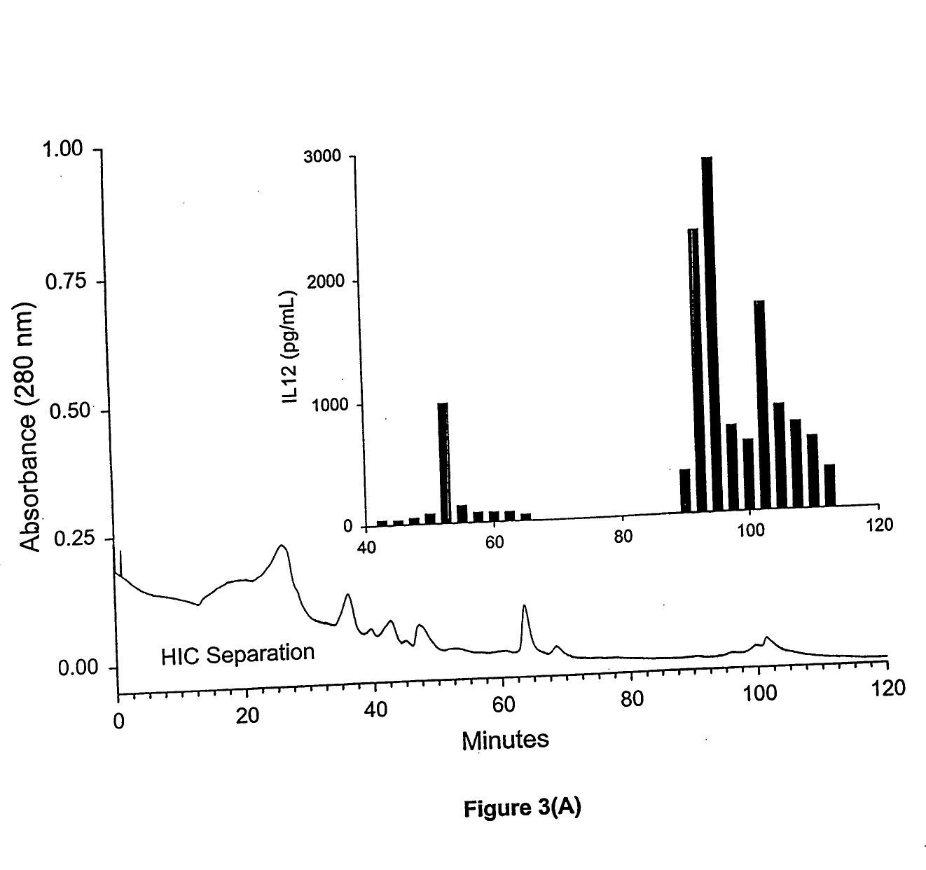

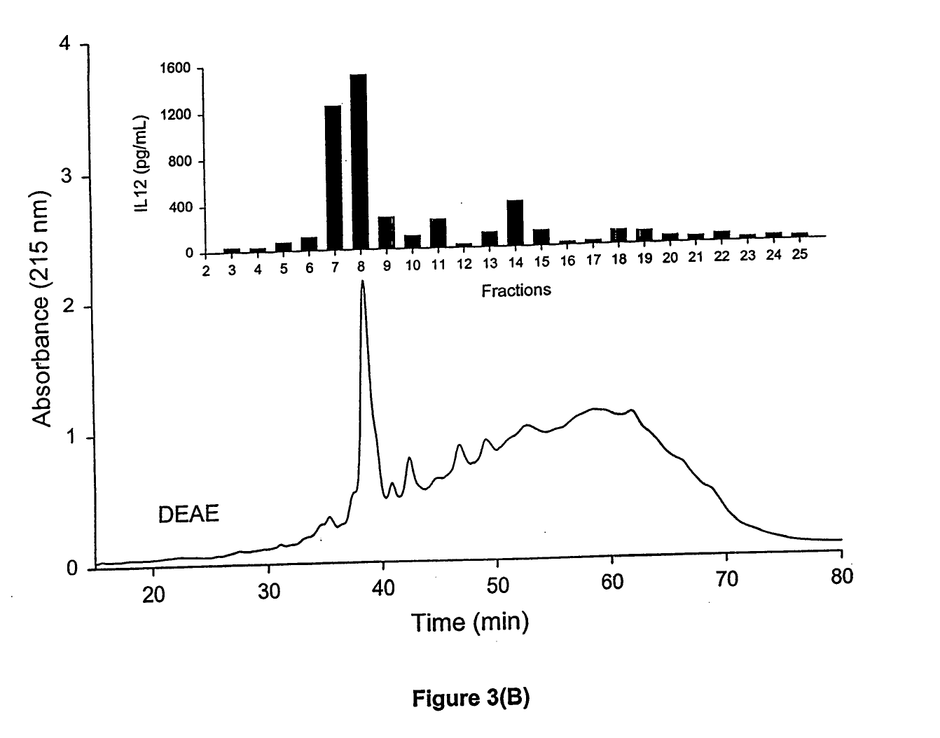

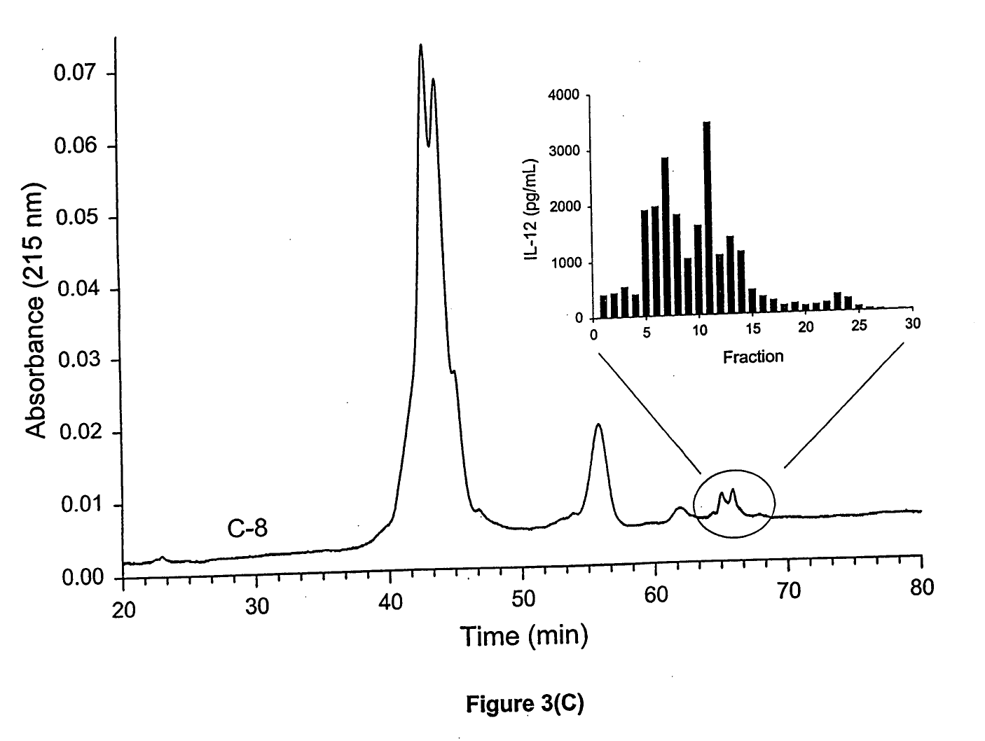

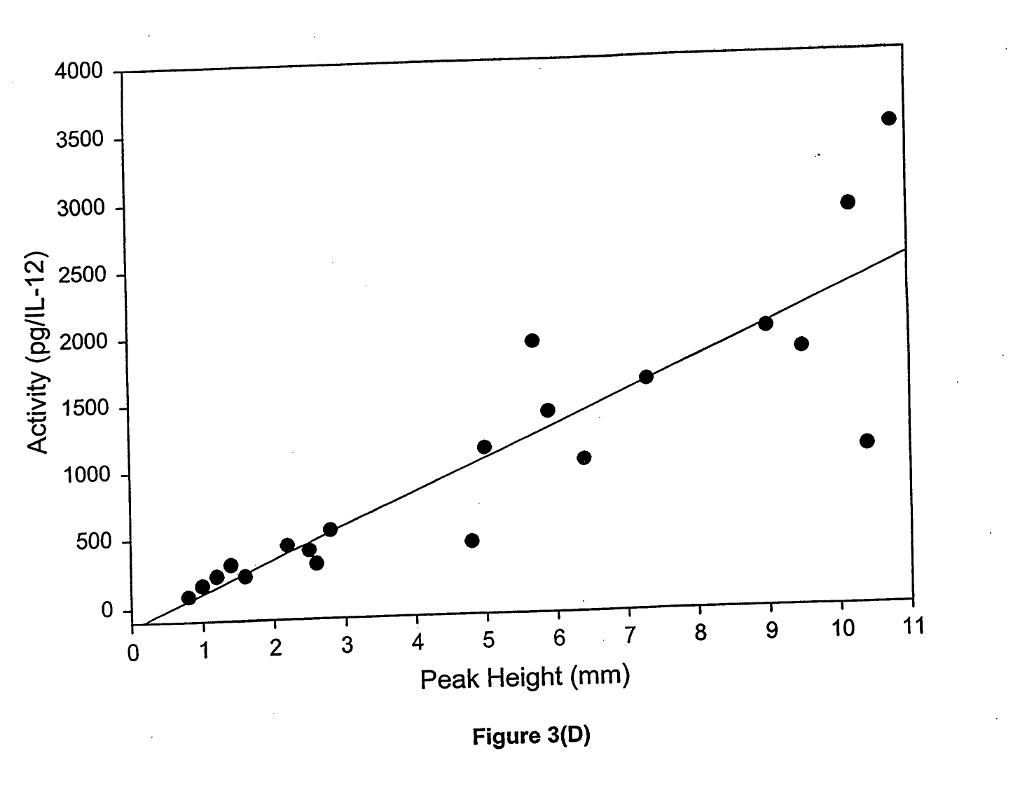

To obtain a purified product, a pool was initially separated on an HPLC HIC column, one-half of the pool at a time. The activity was located near the end of the chromatogram (see FIG. 3(A)), which is indicative of a moderately hydrophobic molecule. Some earlier activity was occasionally detected, but was weak and sporadic. The active fractions were dialyzed to remove ammonium sulfate and were applied to an HPLC DEAE column in 0.15M NaCl. A NaCl gradient was applied and the activity eluted with a major peak near 0.3M ionic strength. The chromatogram and activity are shown in FIG. 3(B). Three DEAE runs were required to process the active material from the HIC. The pooled active region was applied to the C8 reverse phase HPLC column and eluted with an aqueous-acetonitile gradient (FIG. 3(C)). The activity eluted near 40% acetonitrile on a slow, shallow gradient elution protocol. Fourteen separations were performed to process the material from the DEAE separation. The activity was associated with two major neighboring peaks and two or three minor peaks or shoulders. This appeared to represent minor variations in the molecule, but these differences have not been quantified. The question of purity of these active fractions was addressed by plotting activity versus absorbance along a finely-sampled separation. This is shown in FIG. 3(D). The linear regression line is reasonably robust and the intersection near zero indicates the lack of contaminating molecules.

The first major peak, denoted alpha, was obtained from approximately one-half of the C8 separations and was concentrated by roto-evaporation, digested with trypsin, and submitted for peptide separation and Edman sequencing at the MSU Macromolecular Structure Facility. Considerable amount of the protein was apparently lost during the concentrating step. Four peaks generated sequence information, one of them containing two sequences from different regions of the bovine ARP. The information was still sufficient to begin searching database by using PattinProt program (PattinProt searching can be done, e.g., at the website phil.ibcp.fr (select NPS@, and then PattinProt)) and led to identification of ARP as one of the proteins in the mixture. Since the concentrating step involved the multiple transfer of collected fractions into a single tube, we considered that the loss of material must have resulted from protein sticking to the plastic conical tubes. Experiments demonstrated that to move the molecule between tubes required the addition of acetonitrile. Incorporating that methodology change separately on the 2^{nd} half of the alpha peak and the 1^{st} half of the second major peak (beta), generated much higher tryptic peptide quantities. These peptides provided sufficient sequences to extend searching the available public databases (e.g., BLAST, PattinProt) for sequence matches.

### 7. EXAMPLE 2

### Identification of Sporozoite Antigen Protein

Protein sequencing services were provided by the Macromolecular Structure Facility at MSU (Edman procedure) and M-Scan Inc., West Chester, Pa. (MS-MS procedure).

Tryptic peptides for Edman sequencing were prepared using the following procedure. Active fractions, typically 1 mL, from several C8 separations were placed in microcentrifuge tubes and evaporated to near dryness under vacuum (Centrivap Concentrator, Labconco, Kansas City Mo.) at 35° C. All sample tubes were washed three times with 200 μL of 60% HPLC acetonitrile/40% HPLC water/0.1% TFA, each time reducing the volume to near dryness. After the third wash, all fractions were combined into a single microcentrifuge tube and were washed twice with 500 μL of HPLC water again reducing to near dryness after each wash. The combined sample, reduced to approximately 15 μL, was diluted to 125 μL in the digestion buffer (100 mM Tris (reagent grade, Sigma-Aldrich, St. Louis Mo.) pH 8.4, 2 mM CaCl₂). Trypsin (sequence grade, Sigma-Aldrich) was added to 2% (w/w) and the sample incubated at 37° C. for four hours. The pH of the sample was checked and an additional 50 μL of digestion buffer was added. Digestion was halted by flash freezing the sample in liquid nitrogen after 18-20 hours of digestion.

Purification of tryptic peptides was performed at the MSU Macromolecular Structure Facility on a microbore reversed phase HPLC using a Vydac C18 column, 5 μm, 300 Å, ID 800 μm, using a linear gradient from 5% H to 70% H in 160 minutes. G buffer consists of 0.1% TFA in water. H buffer consists of 90% HPLC grade Acetonitrile, 10% HPLC Grade water, and 0.985% TFA. Absorbance was monitored by a Kratos Analytical Spectroflow 783 detector at 214 nm. N-terminal sequencing of selected peptide fragments was performed on a Perkin Elmer/Applied Biosystems 494 cLC Procise Protein Sequencer which utilizes Edman Degradation chemistry.

Samples for MS-MS analysis were isolated and digested as described above. Two samples were sent to M-Scan Inc. (West Chester, Pa.); a Trypsin autodigest and digested sample. Each sample was applied to a C18 ZipTip and desalted by washing with 0.1% aqueous formic acid. Peptides were eluted using a solution of 60% acetonitrile, 40% water containing 0.1% formic acid. The peptide mixture obtained from each sample was analyzed initially by positive ion ESI-MS using a Sciex Q-Star/Pulsar mass spectrometer. The sample was introduced using a nanospray needle and data was collected in the MCA mode. The same instrument and sample introduction method were used for the MS/MS sequencing.

The amino acid sequences from the tryptic peptides, generated by the Edman and MS/MS procedures, are shown in Table 3. Several robust reads were obtained from various peaks using the Edman procedure, although some reads were the convolution of two or more separate sequences and most were contaminated, resulting in multiple reads in almost every position of every peptide. Most commonly used programs, like BLAST, could not perform a search with such complicated data. Therefore, the sequences were assigned by usage of PATTINPROT program (Network Protein Sequence Analysis (NPS@); TIBS 2000, 25:147-150). Multiple amino acid reads at each position were searched by the program at the desired level of similarity (usually higher than 70%), and a list of proteins matching those parameters was generated. Since the list normally contained several dozen of different entries, it was searched again with sequence of a second peptide. This procedure usually produced a list containing only the entries related to sporozoite antigen. In case any identifiable contaminants were on the first list (trypsin, keratin, albumin), their sequences were subtracted from the amino acid reads and the search repeated with the remainder reads. Once the Eimeria sporozoite antigen was considered as a likely candidate, some of the multiple reads were deconvoluted by finding a match for one possible sequence and then determined if the remaining sequence also matched a different part of the protein. The strategy was successful in multiple cases, and resulted in generating several most probably peptide sequences for bovine ARP.

TABLE 3

Probable sequences generated from
Edman tryptic peptides or MS/MS analysis

| | |
|---|---|
| source | sequence |

| | |
|---|---|
| α₁18, α₂18/β₁18¹ | EWLVDTGKVF |
| MS/MS | LVDTGK |
| α₂25 | VFAGGVASIADG |
| α₂25 | AGYQIESVQEDNGTVQ |
| α₂35, α₂32/B₁30, | ³MFGASTDSGGDPNAELVQYN |
| α₁28, β₁32 | |
| MS/MS² | ³MFGASTDS |
| α₂13/β₁14, MS/MS | APDGVYIGGVK |
| α₁18, α₂18/β₁18 | GGGFLIK |
| α₂50/β₁46, α₂49/β₁45 | TPNENIAIALYDEEKEQNKA |
| MS/MS | TPNXXXXIALYDEEKEQNK |
| α₂53/β₁49, α₁40, α₁37 | ADALTTALNFADFLYQ |

¹Reads of combined peptides are marked with “/”, e.g., α₂18/β₁18.
²MS/MS method cannot distinguish between I and L, and often between Q and K, so all the positions with one of those amino acids are not certain from MS/MS reads.
³Since tryptic peptides were used, the position preceeding the first readable amino acid is either R, or K, but it cannot be conclusively determined which one of these two is the correct amino acid.

Possible errors in amino acid assignments cannot be eliminated, however, due to the quality of some of the data. Table 4 lists possible errors (where plain text represents highly confident data, i.e., multiple read and multiple samples; underlined text represents lower confidence; and bold text represents lowest confidence):

TABLE 4
Possible errors in SEQ ID NO: 3, 4, 5, 6 and 7

| | |
|---|---|
| SEQ ID NO:3 | Glu Trp Leu Val Asp Thr Gly Lys Val Phe Ala Gly |
| | Gly Val Ala Ser Ile Ala Asp Gly¹ |
| SEQ ID NO:4 | Arg Met Phe Gly Ala Ser Thr Asp Ser Gly Gly/Asn |
| | Asp Pro Asn Ala Glu Leu Val Lys Ala/Val Gly/Phe |
| | Tyr/Ala Gln/Gly Ile/Gly Glu/Val Ser/Ala Val/Ser |
| | Gln/Ile Glu/Ala Asp Asn/Gly² |
| SEQ ID NO:5 | Gln Ala Ile Val³ |
| SEQ ID NO:6 | Ala Pro Asp Gly Val Tyr Ile Gly Gly Val Lys⁴ |
| SEQ ID NO:7 | Gly/Glu Gly/Trp Gly/Leu Phe/Val Leu/Asp |
| | Ile/Thr Lys/Gly Thr Pro Asn Glu Asn Ile Ala Ile |
| | Ala Leu Tyr Asp Glu Glu Lys Glu Gln Asn Lys Ala |
| | Asp Ala Leu Thr Thr Ala Leu Asn Phe Ala Asp Phe |
| | Leu Tyr Gln⁵ |

¹Lower confidence: two reads convoluted in a single sample. The read with identity to an Eimeria acervulina conserved region is assigned here (see SEQ ID NO:4 for other read).
²Lowest confidence: two reads in one sample. The second choice in each case was assigned to SEQ ID NO:3, due to match in Eimeria. The first choice is assigned here, due to partial match to Eimeria.
³Lowest confidence: Low S/N (signal to noise ratio) and derived from only one sample.
⁴Lower confidence: slightly lower S/N, but no other amino acid reads present for these positions.
⁵Lower confidence: obtained from only one sample, but S/N is good. Lowest confidence: deconvoluted data from two samples containing two sequences. The second choice in each case was assigned to fragment 1, due to match with Eimeria.
The corresponding region in fragment 1 was also corroborated by MS/MS data making it high confidence. The first choice from each double read is assigned here, due to match to Eimeria. This makes the sequence fairely reliable, but the three Gly have a low S/N.

The second half of the beta peak was sent to M-Scan Inc. for MS/MS sequence analysis. Several peptides were identified, and the match to the Eimeria antigen was verified. A rather long variable region was also detected, which still matched the known ARP sequences with low similarity and was therefore assigned to a portion of molecule.

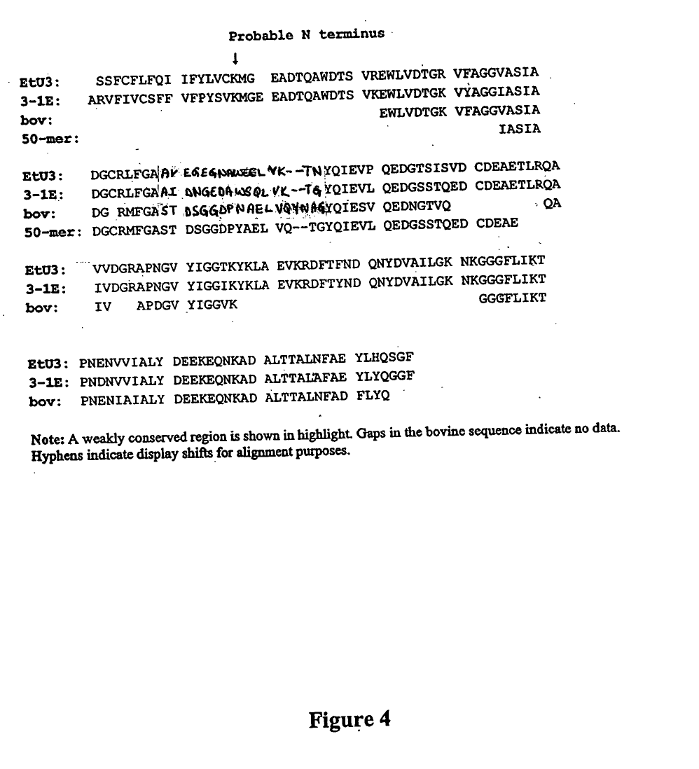

When all peptide sequences were evaluated, evidence for a homologue to the sporozoite antigen was overwhelming. The alignment to the Eimeria 19 Kd antigen 3-1E is shown in FIG. 4. Most of other sequences could be assigned to the understandable contaminants, keratin, trypsin, and bovine albumin, though a small number of the generated sequences remained non-assigned.

### 8. EXAMPLE 3

### Extraction of Activity from E. Tenella Oocysts

Oocysts can be obtained from intentionally infected animals, from barnyard soils, or from feces of infected animal. Methods of isolating oocysts are known in the art. See, e.g., Tomley, Methods: A companion to Methods in Enzymology 13:171-176 (1997), which is incorporated herein in its entirety by reference. For example, one protocol that can be used to obtain sporulated oocysts is as follows: infect 6- to 8-week old Light Sussex chickens by oral dosing with between 10³ and 6×10³ sporulated oocysts and recover oocysts from the ceca 7 days later using either enzymatic or chemical treatment (e.g., remove and cut ceca, add phosphate-buffered distilled water, Ph 8.0, and homogenize to a fine pulp with a commercial blender; add trypsin (Difcon 1:250 powder) to a final concentration of 1.5% w/v; incubate at 41° C. for 30 min, strain through two thicknesses of muslin, and centrifuge at 1000 g for 10 min; discard the supernatant, replace with a similar volume of saturated sodium chloride solution, mix well, and centrifuge again; collect the oocysts from the top of the salt using a syringe fitted with a cannula or a long needle; and wash them repeatedly by centrifugation and resuspension in buffered water) and then sporulate the oocysts (e.g., suspend oocysts in 2% (w/v) potassium dichromate and incubate at 28-30° C. for 48 hours with forced aeration; remove the potassium dichromate by repeated centrifugation and dilution in water; add sodium hypochlorite to a final concentration of 10% (v/v) and incubate on an ice bath for 5-10 min; centrifuge and mix the pellet with saturated sodium chloride solution; overlay the tube contents with distilled water and centrifuge for 5 min; collect clean, bacteria-free oocysts from the interface of the salt and water into sterile distilled water and wash repeatedly by centrifugation into water).

Approximately 3×10⁸ sporulated oocysts (a gift of Dr. Ray Fetterer, USDA) was suspended in 9.0 Ml of PBS and centrifuged for 10 minutes at 200×G. The supernatant (“wash”) was removed and retained. The pellet (˜0.5 Ml packed volume) was suspended in 0.5 Ml 30 Mm Hepes and homogenized on ice with a glass homogenizer for a total of approximately 15 min (prep 1). Most of the oocysts were broken, releasing sporocysts that remained largely intact. A few less rigid, smaller structures (perhaps sporozoites) were observable. Continued homogenization did not appear to appreciably increase the number of these structures. The homogenate was transferred to a microcentrifuge tube and centrifuged for 20 minutes at 6000×G. The supernatant (˜200 μg/Ml protein) was sterile filtered (0.22 μm) and designated the soluble protein extract of E. tenella sporulated oocysts (EtH-1).

In a second preparation, the homogenate was divided into 3 microcentrifuge tubes each with a total volume of approximately 500 μL. Each suspension was sonicated with 30 1-second bursts using a Fisher Scientific Ultrasonic Dismembrator™ (Model 100), with care taken to keep the homogenate cooled during the sonication. Approximately 80% of the released sporocysts were disrupted into non-light refractory “disrupted” sporozoites. The suspensions were pooled and microcentrifuged for 20 minutes at 6000×G. The resulting supernatant was retained, sterile filtered, and designated EtH-2. The pellet was retained and stored at 4° C. for future processing (see 3rd extract).

The starting material for the 3^{rd} extract of Eimeria tenella sporulated oocysts was the residual pellet of the 2nd extract preparation. This pellet was stored at +4° C. for 18 days. The pellet material was suspended in 500 μL 30 Mm Hepes and transferred to a 2 Ml beadbeater tube containing approximately 1.3M1 of 0.5 mm glass beads (BioSpec Products #11079105). The tube was then completely filled with 30 Mm Hepes and processed in a Mini-Beadbeater (BioSpec Products Model #3110BX) for 2 cycles of 3 minutes each at 4800 cpm. The tube was placed on ice for 15 minutes in between the 3 minunte cycles to prevent excessive heating. The suspension was microcentrifuged briefly and the supernatant was separated from the glass beads. This supernatant was microcentrifuged for 20 minutes at 8000×G and was filter-sterilized and tested in the DC/Mil12 release assay for activity.

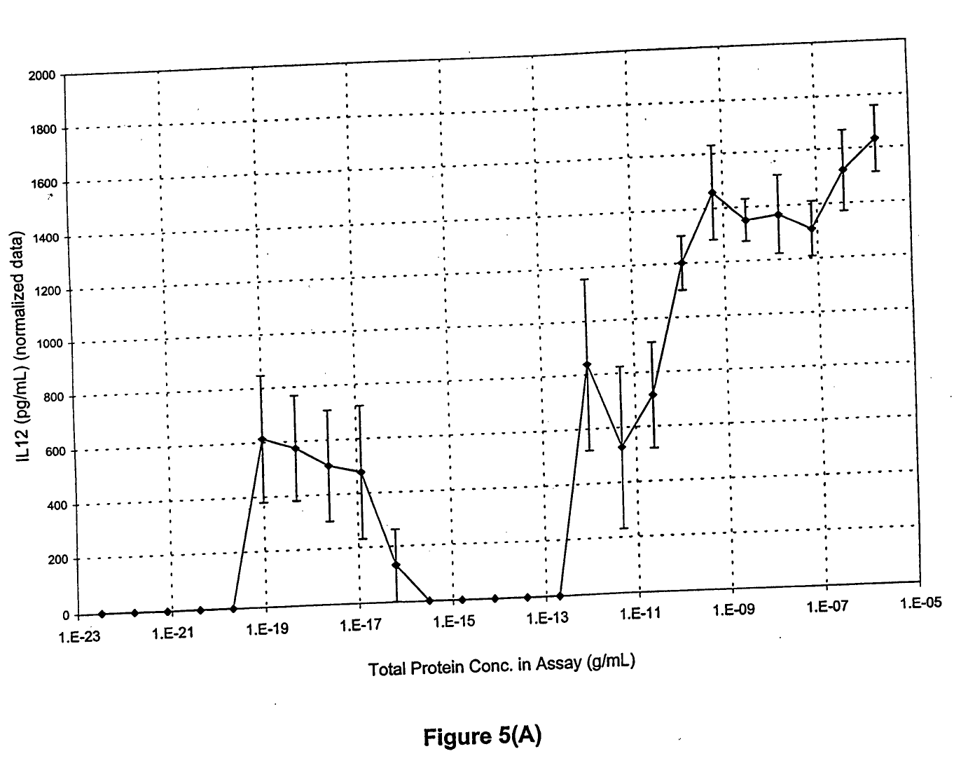

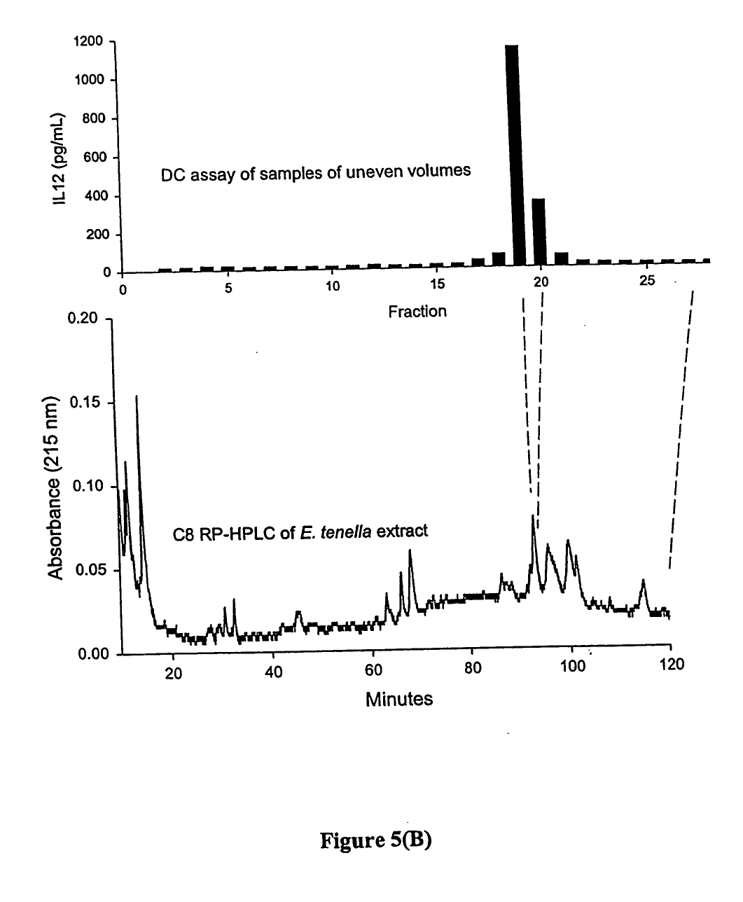

The supernatants from the partially homogenized and washed mixture of broken oocysts, sporocysts, broken sporocysts, and sporozoites contained a large amount of DC activity in a small amount of protein. As shown FIG. 5(A), the first extract supernatant yielded dilution curves that were active to low picogram levels of total protein. For a 20 Kd protein, this is approximately 50 Fm. This assumes that all the protein in the extract is active, which is unrealistic. Therefore, the active molecule is probably active below this level. An unusual second region of activity appeared in all five replicates on two different assay days, which may due to possibility that the active molecule forms inactive complexes (dimer, trimer, multimer) with itself or another molecule, such that its active site is sequestered. Upon sufficient dilution, this complex dissociates and activity reappears.

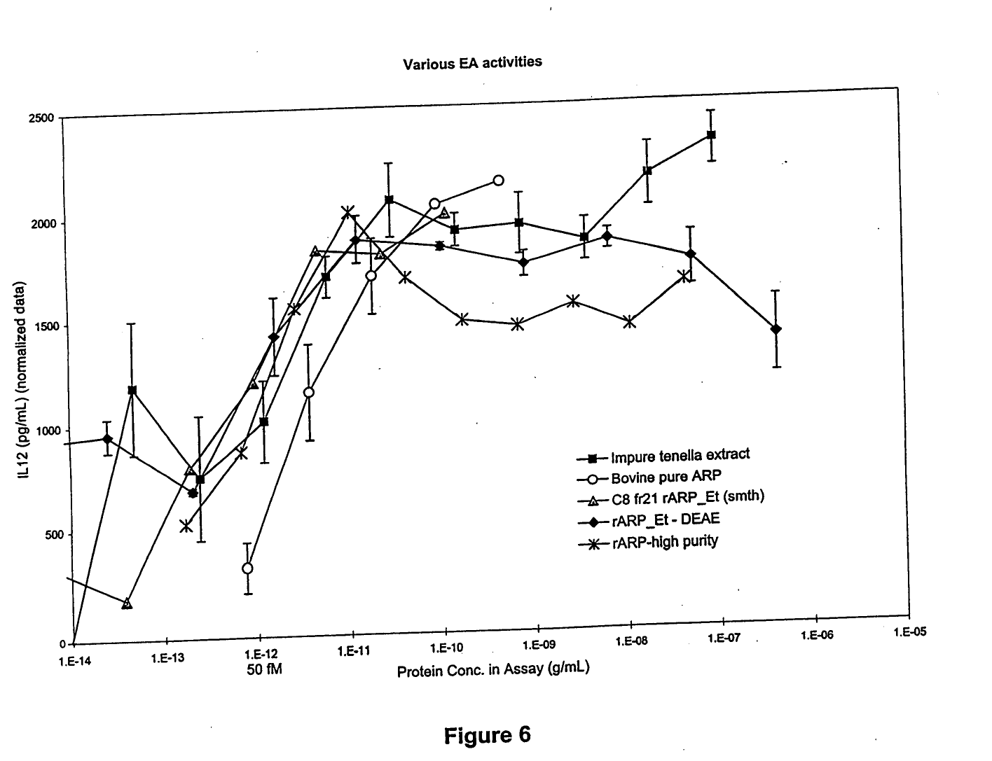

A second extract utilized sonication to facilitate the breakage of oocysts and sporocysts. This did substantially change the yield of active material and there was a repeat of the reappearance of activity at high dilution. A third extract, utilizing the Bead Beater™ to break the cysts with micro beads, generated an anomalously high level of activity in the first DC assay, but subsequent assays were very similar to FIG. 6. The amount of dilution required to reach zero activity is truly remarkable. This extended dose/response may be related to the low dilution “hump” described above. If different size complexes, with different dissociation constants are releasing activity, then this long, flat response may result.

Samples of the extracts were separated on C8 HPLC using the same protocol as for the bovine extract. The chromatogram and activity are shown in FIG. 5 (B). The position of the activity is shifted to higher retention time compared to the highly pure bovine molecule. This appears to indicate a higher hydrophobicity to the Eimeria molecule, when extracted directly from the protozoan.

### 9. EXAMPLE 4

### Crude Membrane Preparation and Purification of the Membrane-Linked Form of ARP from the Sporozoites of E. Tenella

Protocols

(1) Disruption of Oocysts.

1 ml of suspension of oocysts at 3×10⁷/ml in homogenization buffer (HB): 50 Mm Tris Ph 7.4, 1 Mm PMSF, 50 μl/ml protease inhibitors cocktail and 10 Mm ZnCl₂ (in some experiments) was homogenized for 20 minutes in glass homogenizer, followed by 15 freeze-thaw cycles (liquid nitrogen/37° C.) or by sonication (Fisher Scientific Ultrasonic Dismembrator, model 100) 10 seconds×36-45 bursts on ice with three freeze-thaw cycles. The homogenization resulted in almost complete oocyst breakage into sporocysts. Sonication resulted in almost complete breakage of all structures present except sporocyst walls.

(2) Membrane Sedimentation and Washing.

The cell homogenate was spun at 15000 g 15 min, 4° C. to pellet membranes. This centrifugation regime is sufficient to pellet also nuclei, other organelles, big ARP aggregates and storage granules, if any. Organelles should be disrupted in further purification cycles in HB without ZnCl₂ Pellet was suspended in 1 ml of HB and centrifuged again. Centrifugation/resuspension was repeated once more with and 2-3 times more without 10 Mm ZnCl₂ in the suspension buffer. The pellet obtained by this procedure with two washing cycles in the presence of 10 Mm ZnCl₂ was used as a crude membrane preparation in immune-stimulatory activity test.

(3) Release of ARP from the Membranes.

For purification of the membrane-linked ARP, the final pellet was suspended in 100 μl of HB without ZnCl₂ and incubated at 37° C. 1-4 hours with or without protease inhibitors and phospholipase C from Bacillus cereus, 16 U/ml.

(4) Membrane Sedimentation and Washing.

ARP released into solution was separated from the insoluble material by centrifugation as described in step 2, followed by two washing cycles. All three supernatants were combined.

(5) C8 Reverse Phase Chromatography.

C8 chromatography was performed as described in Section 6 with linear gradient from 100% buffer E to 100% buffer F in 90 minutes. An aliquot from each fraction was assayed for DC activity after renaturing (FIG. 11).

### CONCLUSIONS

It was determined that in control samples (without PLC) significant amount of DC-factor is released into solution spontaneously. Upon addition of external PLC the amount of released activity increased in all four experiments, the increase ranging from 10-fold to 25%, the data suggesting that the DC-factor is indeed GPI linked to the outer cell membrane.

Material released into solution spontaneously as well as after PLC digestion was pure enough to be analyzed by reverse phase HPLC chromatography. The region where the DC-activity eluted (41-55% buffer F) was quite clean, containing 2-3 peaks with UV absorbance at 215 nm below 0.01. The position of DC-activity on the gradient was the same for all samples analyzed: supernatants generated by membrane washing in step 2 and supernatants generated by membrane washing in step 4 after incubation at 37° C. for 4 hrs with or without PLC.

The presence of 10 Mm ZnCl₂ in the oocyst suspension during sonication and membrane washing in step 2 was shown to significantly inhibit the ARP loss in the first two washing steps, the effect, probably, being due to inhibition of the internal phospholipase and to a smaller extent—to aggregation of ARP. Less than 1% ARP loss was obtained in the presence of ZnCl₂ as opposed to ca. 50% loss in the absence of ZnCl₂. This allows to wash away the intracellular material generated by cell breakage thus obtaining a clean membrane preparation without any loss of DC-factor (ARP) (presence of ARP aggregates in this preparation is possible). Removal of ZnCl₂ by resuspending the membrane pellet in ZnCl₂-free buffer restores the spontaneous activity release. By washing the pellet in ZnCl₂-free buffer combined with incubation at 37° C. for 1 hr about 35-50% of DC-activity of the pellet can be removed into solution, thus producing a clean preparation of DC-factor (ARP). The whole procedure takes one day.

### 10. EXAMPLE 5

### Effect of E. Tenella Sporulated Oocyts on HIV-1 Replication in an IN VIVO Hollow Fiber Mouse Model

This example demonstrates the effect of the protein extract of E. tenella sporulated oocysts compared to a positive control antiviral compound (3′-Azido-3′-deoxthymidine; AZT) and a no-drug control (virus control) on HIV-1 replication in an in vivo hollow fiber mouse model. In this model, CEM-SS cells are infected with HIV-1_{IIIB}, sealed in hollow fibers, followed by implantation of the fibers into both the subcutaneous and intraperitoneal compartments of the mice. Mice are dosed daily with the E. tenella sporulated oocyst protein extract, AZT, or control buffer for one week. After one week of treatment, the hollow fibers from each compartment of the mice, blood serum samples and a wash of the peritoneal cavity from each mouse are collected. A number of analyses are performed on these samples to analyze the effect of E. tenella sporulated oocyst protein extract on HIV-1 replication.

### 10.1 Materials and Methods

Drug Preparation:

Approximately 3×10⁸ sporulated oocysts (a gift from Dr. Ray Fedderer at the U.S.D.A.) were suspended in 9.0 mL of PBS and centrifuged for 10 mins at 200×G. The supernatant (“wash”) was removed and retained. The pellet (approximately 0.5 mL packed volume) was suspended in 0.5 mL 30 mM Hepes and homogenized on ice with a glass homogenizer for a total of approximately 15 min. Most of the oocysts were broken, releasing sporocysts that remained largely intact. A few less rigid, smaller structures (perhaps sporozoites) were observable. Continued homogenization did not appear to appreciably increase the number of these structures. The homogenate was divided into 3 microcentrifuge tubes each with a total volume of approximately 500 μL. Each suspension was sonicated with a 30 1-second bursts using a Fisher Scientific Ultrasonic Dismembratorm (Model 100), with care taken to keep the homogenate cooled during the sonication. Approximately 80% of the released sporocysts were disrupted into non-light refractory “disrupted” sporozoites. The suspensions were pooled and microcentrifuged for 20 min at 6000×G. The resulting supernatant was retained and sterile filtered. Three different concentrations of this supernatant was used as the test agent in this experiment. The approximate amounts of the “active protein” in the test agent were 0.7 ng (concentration #3), 7 ng (concentration #2) and 70 ng (concentration #1). The positive control antiviral compound 3′-Azido-3′-deoxythymidine (AZT) was purchased from Sigma (St. Louis, Mo.) and solubilized at the appropriate concentration using 30 mM Hepes solution. 30 mM Hepes solution (used to solubilize the oocysts) was utilized in dosing negative control animals. All test agents were stored at −20° C. until use. At the time of use, single use aliquots of the test agents were removed from the freezer and allowed to thaw at room temperature in preparation for dosing of animals.

HIV-1 Hollow Fiber Mouse Model:

The human T4-lymphoblastoid cell line CEM-SS was obtained courtesy of Dr. Peter Nara through the NIAID AIDS Research and Reference Reagent Program (reagents are available to qualified researchers from government laboratories). CEM-SS cells were propagated in T-75 flasks in RPMI 1640 medium (no phenol red) supplemented with 10% Fetal Bovine Serum (heat inactivated), 2 mM L-glutamine, 100 U/mL penicillin, 100 μg/mL streptomycin, and 10 μg/mL gentamycin. On the day preceding the assay, the cells were split 1:2 to assure they were in an exponential growth phase at time of infection. The laboratory adapted, T-cell tropic HIV-1_{IIIB} late was obtained courtesy of Dr. Robert Gallo through the NIAID AIDS Research and Reference Reagent Program. Pre-titered aliquots of virus were removed from the freezer (−80° C. or LN₂) and thawed rapidly to room temperature in a biological safety cabinet immediately before use. CeIlMaxg Implant Membranes (molecular weight cutoff=500 kD; “hollow fibers”) were obtained from Spectrum (Laguna Hills, Calif.). Hollow fibers were prepared for use by soaking in 70% ethanol for a minimum of 72 hours followed by rinsing with sterile-filtered distilled water, autoclaving and storage in sterile water at 4° C. until use. MTT [3-(4,5-Dimethyl-2-thiazolyl)-2,5-diphenyl-2H-tetrazolium bromide] and Protamine Sulfate were purchased from Sigma (St. Louis, Mo.). MTT was solubilized as a stock solution at 5 mg/ml in Phosphate Buffered Saline. Protamine Sulfate was prepared as a 2.5% solution in normal 0.9% saline. HIV-1 p24 (Capsid) ELISA kits were purchased from Coulter Electronics. HIV-1 Reverse Transcriptase Assay reagents were purchased from NEN (now PerkinElmer Life Sciences: Boston, Mass.) and Amersham/Pharmacia (Piscataway, N.J.) as indicated below in the assay description. BALB/C mice (female, 15-18 grams) were purchased from the National Cancer Institute breeding facility (Fort Detrick, Frederick, Md.). Mice were quarantined and acclimated to the facility for a minimum of 7 days prior to use in the experiments.

Animal Use and Veterinary Care:

The care and husbandry of animals was performed in compliance with all federal, state and local laws and guidelines as well as in accordance with recommendations provided in the NIH Guide and other accepted standards of laboratory animal care and use. The animal studies were performed by scientific personnel and animal technicians at an animal facility that: 1) maintains an Animal Welfare Assurance with the Public Health Service, Office of Laboratory Animal Welfare; 2) is a USDA registered Class R Research Facility; and 3) is an AAALAC fully accredited institution. The animal facility has an Attending Veterinarian who is employed full time and is responsible for all aspects of veterinary care for animals used in research at the facility.

The Institute Animal Care and Use Committee (IACUC) is responsible for the oversight and evaluation of animal care and use in the facility. Its responsibilities include: inspection of facilities, evaluation of program and animal-activity areas, submission of reports to responsible institutional officials, review of proposed uses of animals in research, and review of concerns involving animal care and use. The HIV-1 hollow fiber mouse model studies described herein were performed under the IACUC approved animal care and use protocol entitled “In Vivo AIDS/HBV/HCV Drug Testing”. This ACUP was reviewed and approved by the IACUC.

Evaluation of Antiviral Efficacy in the HIV-1 Hollow Fiber Mouse Model:

The HIV-1 hollow fiber mouse model was performed (Hollingshead et al., Antiviral Res. 1995, 28:265-279; Buckheit et al., AIDS Res. Hum. Retroviruses 1997, 13:789-796; Xu et al., Bioorg. Med. Chem. Lett. 1999, 9:133-138; Quenelle et al., Antiviral Res. 1997, 35:123-129). In brief, CEM-SS cells were infected with HIV-1_{IIIB} at a multiplicity of infection (MOI) of 0.0002 and sealed in hollow fibers (2×10⁵ cells/fiber) immediately prior to implantation in the mice. Cell control hollow fibers (mock infected) and blank fibers (containing tissue culture medium without cells or virus) were prepared in a similar manner. Hollow fibers were implanted in the subcutaneous and intraperitoneal compartments of the mice (three fibers/compartment/mouse). Mice were treated with the E. tenella protein extract once daily (0.1 mL/10 g body weight) on day 0 (immediately prior to implantation of the fibers) and on days 1-6. Cell control, virus control and blank control mice were dosed similarly using 30 mM Hepes. AZT treated mice were also dosed similarly, however, dosing was performed twice daily at a concentration of 150 mg/kg (300 mg/kg/day). On day seven post implantation, the fibers, blood samples and peritoneal wash were collected from the mice. The viability of the infected CEM-SS cells collected from hollow fibers was determined using an MTT dye conversion assay. Viral reverse transcriptase assay and p24 (capsid) antigen quantification were performed on the hollow fiber cultures as well as the blood samples and the peritoneal wash. Of the three fibers collected from each compartment (intraperitoneal and subcutaneous), one fiber was used for analysis of viral reverse transcriptase and p24, while the remaining two fibers used to analyze cell viability by MTT dye reduction. The experimental treatment groups used in the study included the following:
1. E. tenella protein extract concentration #1 (virus infected fibers; 7 mice)
2. E. tenella protein extract concentration #2 (virus infected fibers; 7mice)
3. E. tenella protein extract concentration #3 (virus infected fibers; 7 mice)
4. AZT; positive control antiviral drug (virus infected fibers; 7 mice)
5. Virus Control, no drug treatment (treated with vehicle; virus infected fibers; 7 mice)
6. Cell Control, no drug treatment (treated with vehicle; mock-infected fibers; 7 mice)
7. Blank Control, no drug treatment (treated with vehicle; “empty” fibers; 2 mice)
   MTT Staining for Cell Viability:

The viability of cells in the hollow fiber cultures was measured using an MTT dye reduction assay. MTT is a tetrazolium dye that is metabolized by the mitochondria enzymes of metabolically active cells to yield a soluble formazan product that can be detected spectrophotometrically. Following collection from the animals, hollow fibers were placed individually into the wells of a 6-well tissue culture plate containing 2 mL of pre-warmed (37° C.) RPMI medium (without phenol red) supplemented with 20% fetal bovine serum. Cells were assayed to viability by adding 180 μL of MTT stock solution (pre-warmed to 37° C.) to each of the wells, followed by incubation for 4 hours at 37° C. in a 5% CO₂ atmosphere, removal of medium/MTT solution, and addition of 4 mL of 2.5% Protamine Sulfate (PMS). Fibers were stored at 4° C. in PMS for 24 hours after which fibers were collected, wiped with gauze to remove stained debris from the exterior of the fibers, transferred to the wells of a 24-well plate and allowed to dry for 24 hours in a biological safety cabinet. After drying, fibers were cut in half and 250 μL of Dimethyl Sulfoxide (DMSO) was added to each well/fiber followed by incubation (protected from light) for 4 hours at room temperature on a rocker. Subsequently, 150 μL of each sample was transferred to a 96 well plate and the optical density of the samples read at 570/650 nm using a Molecular Devices Vmax or SpectraMaxPlus plate reader.

p24 Antigen ELISA:

Following collection of the fibers from the mice, detection of p24 antigen was performed according to the manufacturer's instructions. Individual fibers were cut open and placed in Eppendorf tubes and cells lysed by the addition of 250 μl of lysis buffer (0.02 M Tris-HCl pH 7.5, 0.05 M NaCl, 0.5% Triton X-100), incubation at room temperature for 30 minutes, vortexing, followed by centrifugation at 14,000 rpm for four minutes in a microcentrifuge. Serum and peritoneal wash samples were clarified by centrifugation without further manipulation prior to analysis. All p24 determinations on the resulting supernatants were performed following serial dilution of the samples to ensure absorbances in the linear range of the standard p24 antigen curve. The standard curve is produced using manufacturer-supplied standards and instructions. Data are obtained by spectrophotometric analysis at 450/570 nm using a Molecular Devices Vmax or SpectraMaxPlus plate reader. Final concentrations are calculated from the optical density values using the Molecular Devices SoftMax Pro software package and expressed in pg/ml p24 antigen.

Reverse Transcriptase Assay:

A microtiter plate-based reverse transcriptase (RT) reaction was utilized (Buckheit et al., AIDS Research and Human Retroviruses 1991, 7:295-302). Tritiated thymidine triphosphate (³H-TTP 80 Ci/mmol, NEN) was received in 1:1 dH₂O: Ethanol at 1 mCi/mL. Poly rA:oligo dT template:primer (Pharmacia) was prepared as a stock solution by combining 150 μL poly rA (20 mg/mL) with 0.5 mL oligo dT (20 units/mL) and 5.35 mL sterile dH₂O followed by aliquoting (1.0 mL) and storage at −20° C. The RT reaction buffer was prepared fresh on a daily basis and consisted of 125 μL 1.0 M EGTA, 125 μL dH₂O, 125 μL 20% Triton X100, 50 μL 1.0 M Tris (pH 7.4), 50 μL 1.0 M DTT, and 40 μL 1.0 M MgCl₂. The final reaction mixture was prepared by combining 1 part ³H-TTP, 4 parts dH₂O, 2.5 parts poly rA:oligo dT stock and 2.5 parts reaction buffer. Ten microliters of this reaction mixture was placed in a round bottom microtiter plate and 15 μL of supernatant resulting from the lysis of cells collected from the fibers (see the section describing the p24 Antigen ELISA above) or serum or peritoneal wash (serum and peritoneal wash samples were clarified by centrifugation without further manipulation prior to analysis) was added and mixed. The plate was incubated at 37° C. for 60 minutes. Following incubation, the reaction volume was spotted onto DE81 filter-mats (Wallac), washed 5 times for 5 minutes each in a 5% sodium phosphate buffer or 2×SSC (Life Technologies), 2 times for 1 minute each in distilled water, 2 times for 1 minute each in 70% ethanol, and then dried. Incorporated radioactivity (counts per minute, CPM) was quantified using standard liquid scintillation techniques.

Data Analysis:

Data analysis and graphing was performed using Microsoft Excel. Raw data are provided in table format.

### 10.2 Results

Observations:

There was no apparent toxicity resulting from any of the treatments used in the study; this is consistent with clinical trials conducted for cancer treatment, in which the extract has proven exceedingly safe with no significant side effects.

Three animals were found dead during the course of the experiments due to causes unrelated to the treatment or toxicity. One animal from the BR1 Concentration #2 and one animal from the Cell Control Groups were found dead the day following fiber implantation due to surgical complications (mice chewed away the intraperitoneal incision closing staples). The third mouse, from the BR1 Concentration #1 treatment group, was found dead on day 4 of the study of unknown causes. This animal death was most likely caused by complication due to repeated Intraperitoneal injections (i.e. needle accidentally hitting organ or blood vessel).

MTT Assay Results:

Hollow fibers were analyzed using this assay to determine the viability of the cells contained in the fibers. HIV-1_{IIIB} replication is cytopathic in CEM-SS cells and therefore kills the cells as the virus spreads through the culture. Inhibition of this cytopathic effect, and thus increase in the number of viable cells is indicative of an antiviral effect. Data from intraperitoneal and subcutaneous fibers are summarized in Tables 5 and 6.

TABLE 5
MTT Intraperitoneal Fiber Data Summary

| | | | % CPE Reduction | |
|---|---|---|---|---|
| Treatment Group | Average | StDev | % Value | % StDev |
| BRI Conc. 1 | 0.84 | 0.05 | 0.00 | 6.49 |
| BRI Conc. 2 | 0.86 | 0.18 | 0.00 | 23.01 |
| BRI Conc. 3 | 1.20 | 0.18 | 3.16 | 23.46 |
| AZT | 1.11 | 0.10 | 0.00 | 12.94 |
| Virus Control | 1.17 | 0.32 | 0.00 | 41.56 |
| Cell Control | 1.95 | 0.22 | 100.00 | 28.55 |
| Blank | 0.42 | 0.09 | 0.00 | 12.02 |

TABLE 6
MTT Subcutaneous Fiber Data Summary

| | | | % CPE Reduction | |
|---|---|---|---|---|
| Treatment Group | Average | StDev | % Value | % StDev |
| BRI Conc. 1 | 0.81 | 0.20 | 0.00 | 13.48 |
| BRI Conc. 2 | 0.88 | 0.26 | 0.82 | 17.80 |
| BRI Conc. 3 | 0.99 | 0.14 | 8.43 | 9.62 |
| AZT | 0.96 | 0.26 | 6.83 | 18.08 |
| Virus Control | 0.86 | 0.12 | 0.00 | 8.30 |
| Cell Control | 2.33 | 0.22 | 100.00 | 15.23 |
| Blank | 0.34 | 0.06 | 0.00 | 3.76 |

Reverse Transcriptase Assay Results:

Hollow fibers were analyzed for HIV-1 reverse transcriptase activity (Tables 7, 8, 9, and 10). As HIV-1 replicates in the CEM-SS cells, the amount of reverse transcriptase protein present in the culture increases overtime. Inhibition of the production of this protein is indicative of an antiviral effect (Tables 7 and 8). The detection of reverse transcriptase in the peritoneal wash (Table 8) and serum (Table 9) samples represents the release of virus or viral proteins form the infected CEM-SS cells, through the hollow fiber and into the peritoneal cavity and circulatory system of the mouse. A decrease in the amount of this protein found in the blood and peritoneal cavity is therefore also indicative of an antiviral effect.

TABLE 7
Reverse Transcriptase Transcript
Subcutaneous Fiber Data Summary

| | | | % Virus Control | |
|---|---|---|---|---|
| Treatment Group | Average | StDev | % Value | % StDev |
| BRI Conc. 1 | 11751 | 1238 | 83.1 | 8.81 |
| BRI Conc. 2 | 11398 | 655 | 80.6 | 4.66 |
| BRI Conc. 3 | 11612 | 1065 | 82.1 | 7.57 |
| AZT | 10178 | 2160 | 71.9 | 15.36 |
| Virus Control | 14124 | 1379 | 100.0 | 9.81 |
| Cell Control | 61 | 7 | 0.0 | 0.05 |
| Blank | 36 | 8 | | |

TABLE 8
Reverse Transcriptase Intraperitoneal Fiber Data Summary

| | | | % Virus Control | |
|---|---|---|---|---|
| Treatment Group | Average | StDev | % Value | % StDev |
| BRI Conc. 1 | 12941 | 1866 | 108.2 | 15.71 |
| BRI Conc. 2 | 15070 | 1480 | 126.1 | 12.46 |
| BRI Conc. 3 | 9191 | 1155 | 76.6 | 9.73 |
| AZT | 6372 | 676 | 52.8 | 5.69 |
| Virus Control | 11971 | 1752 | 100.0 | 14.75 |
| Cell Control | 96 | 12 | 0.0 | 0.10 |
| Blank | 68 | 6 | | |

TABLE 9
Reverse Transcriptase Peritoneal Wash Data Summary

| | | | % Virus Control | |
|---|---|---|---|---|
| Treatment Group | Average | StDev | % Value | % StDev |
| BRI Conc. 1 | 731 | 107 | 65.5 | 12.58 |
| BRI Conc. 2 | 705 | 108 | 62.4 | 12.65 |
| BRI Conc. 3 | 419 | 43 | 28.8 | 5.06 |
| AZT | 392 | 112 | 25.7 | 13.18 |
| Virus Control | 1025 | 97 | 100.0 | 11.42 |
| Cell Control | 174 | 10 | 0.0 | 1.14 |
| Blank | 133 | 3 | | |

TABLE 10
Reverse Transcriptase Serum Data Summary

| | | | % Virus Control | |
|---|---|---|---|---|
| Treatment Group | Average | StDev | % Value | % StDev |
| BRI Conc. 1 | 488 | 150 | 112.0 | 103.50 |
| BRI Conc. 2 | 282 | 36 | 0.0 | 24.81 |
| BRI Conc. 3 | 423 | 236 | 66.6 | 163.44 |
| AZT | 403 | 52 | 52.8 | 36.11 |
| Virus Control | 471 | 43 | 100.0 | 29.99 |
| Cell Control | 326 | 39 | 0.0 | 27.03 |
| Blank | 218 | 8 | | |

p24 Assay Results:

Hollow fibers were analyzed for p24 antigen (viral capsid protein) content. As HIV-1 replicates in the CEM-SS cells (Tables 11 and 12), the amount of this protein present in the culture increases overtime. Inhibition of production of this protein is indicative of an antiviral effect. The detection of p24 in the peritoneal wash (Table 13) and serum (Table 14) samples represents the release of virus or viral proteins form the infected CEM-SS cells, through the hollow fiber and into the peritoneal cavity and circulatory system of the mouse. A decrease in the amount of this protein found in the blood and peritoneal cavity is therefore also indicative of an antiviral effect.

TABLE 11
p24 Intraperitoneal Fiber Data Summary

| | | | % Virus Control | |
|---|---|---|---|---|
| Treatment Group | Average | StDev | % Value | % StDev |
| BRI Conc. 1 | 161133 | 5546 | 74.2 | 2.56 |
| BRI Conc. 2 | 195085 | 13485 | 89.9 | 6.21 |
| BRI Conc. 3 | 233281 | 3732 | 107.5 | 1.72 |
| AZT | 215136 | 6603 | 99.1 | 3.04 |
| Virus Control | 217059 | 36828 | 100.0 | 16.97 |
| Cell Control | 0.07 | 0.02 | 0.0 | 0.00 |
| Blank | 0.09 | 0.00 | | |

TABLE 12
p24 Subcutaneous Fiber Data Summary

| | | | % Virus Control | |
|---|---|---|---|---|
| Treatment Group | Average | StDev | % Value | % StDev |
| BRI Conc. 1 | 221041 | 7440 | 78.0 | 2.63 |
| BRI Conc. 2 | 102614 | 3818 | 36.2 | 1.35 |
| BRI Conc. 3 | 255229 | 2774 | 90.1 | 0.98 |
| AZT | 312334 | 8523 | 110.3 | 3.01 |
| Virus Control | 283272 | 5429 | 100.0 | 1.92 |
| Cell Control | 0.15 | 0.01 | 0.0 | 0.00 |
| Blank | 0.04 | 0.01 | | |

TABLE 13
p24 Peritoneal Wash Data Summary

| | | | % Virus Control | |
|---|---|---|---|---|
| Treatment Group | Average | StDev | % Value | % StDev |
| BRI Conc. 1 | 90 | 10 | 77.2 | 8.90 |
| BRI Conc. 2 | 119 | 4 | 101.9 | 3.13 |
| BRI Conc. 3 | 69 | 5 | 58.6 | 4.64 |
| AZT | 109 | 12 | 92.8 | 10.17 |
| Virus Control | 117 | 9 | 100.0 | 7.82 |
| Cell Control | 1 | 0 | 0.0 | 0.15 |
| Blank | 0 | 0 | | |

TABLE 14
p24 Serum Data Summary

| | | | % Virus Control | |
|---|---|---|---|---|
| Treatment Group | Average | StDev | % Value | % StDev |
| BRI Conc. 1 | 15 | 5 | 31.4 | 11.35 |
| BRI Conc. 2 | 18 | 1 | 36.9 | 3.17 |
| BRI Conc. 3 | 11 | 2 | 23.3 | 3.51 |
| AZT | 18 | 11 | 37.0 | 22.59 |
| Virus Control | 47 | 5 | 100.0 | 10.30 |
| Cell Control | 1 | 0 | 0.0 | 0.08 |
| Blank | 0 | 0 | | |

### 10.3 Discussion

The results of this experiment indicate that the protein extract of E. tenella sporulated oocysts has antiviral activity against HIV.

Overall, the results show reduction of viral reverse transcriptase and p24 antigen content in the peritoneal wash and serum samples upon treatment with the protein extract of E. tenella sporulated oocysts at three different concentrations. More specifically, a reduction in viral reverse transcriptase activity in the peritoneal wash was observed at all three concentrations, with the greatest reduction observed at concentration #3 which was comparable to the effect of the positive control compound AZT (Table 7). A reduction in viral p24 antigen content was observed at concentrations #1 and #3, with the greatest reduction again observed at concentration #3. These activities were greater that those observed with AZT, which appeared not to have an effect on this viral endpoint analysis (Table 11). A reduction in viral reverse transcriptase activity in the serum samples was observed at concentrations #2 and #3, this time with the greatest reduction at concentration #2. This reduction was greater than that observed with AZT. Moreover, the apparent reduction at concentration #3 did not appear to be significant due to a wide range of variability in the samples for this treatment group (Table 8). A reduction in viral p24 antigen content was observed in the serum samples at all concentrations tested, with the greatest reduction observed at concentration #3. At all concentrations, the effect was equivalent to or greater than that of AZT (Table 12).

The results also illustrate reduction of viral reverse transcriptase and p24 antigen content in hollow fibers collected from the intraperitoneal and subcutaneous compartments of the mice upon treatment with the protein extract of E. tenella sporulated oocysts at three different concentrations. More specifically, a reduction in viral reverse transcriptase activity was observed in the fibers collected from the intraperitoneal compartment in mice treated with concentration #3. This reduction was less than observed with AZT (Table 9). A reduction in viral p24 antigen content was observed in the fibers collected from the intraperitoneal compartment in mice treated with concentration #1. This reduction was greater than that observed with AZT, which appeared not to have an effect on this viral endpoint analysis (Table 13). A slight to moderate reduction in viral reverse transcriptase activity was observed in the fibers collected from the subcutaneous compartment in mice treated with all concentrations. This effect at all concentrations was comparable to AZT (Table 10). A slight to moderate reduction in viral p24 antigen content was also observed in the fibers collected from the subcutaneous compartment in mice treated with all concentrations. The greatest reduction was observed at concentration #2, however all concentrations had an effect greater than that of AZT, which appeared not to have an effect on this viral endpoint analysis (Table 11).

The protein extract of E. tenella sporulated oocysts did not appear to inhibit the cytopathic effect (CPE) of HIV-1_{IIIB} on CEM-SS cells as none of the concentrations tested reduced the viral induced CPE observed in the hollow fiber cultures. Similarly, AZT did not have an effect on this viral endpoint (Tables 5 and 6). Therefore, similar to AZT, the protein extract of the E. tenella sporulated oocysts appeared to have its greatest effect on the release of virus or viral proteins from the implanted cultures. This could be indicative of an antiviral effect on the replication of HIV-1 in the cultures, or it may be an effect on the immune system of the animals, causing the virus or viral proteins to be recognized more readily and cleared by the immune response in the animals.

### 11. EXAMPLE 6

### Gene Cloning and Expression of Emeria Tenella cDNA

### 11.1 Cloning and Expression of E. Tenella ARP

Total RNA was obtained from E. tenella by treating 107 oocysts to Proteinase K™ digestion (Sigma) at a final concentration of 1 mg/mL, in 200 μL of buffer containing 50 mM Tris-HCl pH 7.5, 10 mM EDTA, 1M guanidine thiocyanate (Sigma), and 0.5% of N-lauroylsarcosine (Sigma). The mix was shaken for 16 hr at 37° C. in a sealed Eppendorf™ tube followed by addition of 120 mg of guanidine thiocyanate, and 1 ml of TRI™ reagent (MRC, Inc.). Isolation of total RNA was performed according to the manufacturer recommendations based on Chomczynsky and Sachi (1987). The final pellet of total RNA was washed twice with 75% ethanol, air dried, and dissolved in 20 μL of sterile distilled, deionized water (DDW) pre-treated with 0.4% diethyl pyrocarbonate (DEP) to inhibit RNases.

The first strand cDNA was obtained with SuperScript RT™ (Life Technologies) in accordance with manufacturer recommendations. About 1 μg (6 μL) of total RNA, and 1 μg of oligo(dT₂O) (Genemed) were mixed together in total volume 11 μL (adjusted with DEP-treated sterile DDW), heated at 90° C. for 2 min, and placed on ice. To the mixture, 4 μL of 5× first strand buffer, 2 μL of 0.1 M DTT, 1 μL of a mixture of all four dNTP's (at 25 mM each, Life Technologies) and 30 U of anti RNase (Ambion) were added. This was incubated for 2 min at 37° C. followed by addition of 200 U of SuperScript R™ and a brief low-speed centrifugation. The reaction proceeded for 100 min at 37° C. followed by 15 min at 70° C. to inactivate the enzyme. RNase H (4 U, Life Technologies) was added for 30 min at 37° C. to digest the RNA from the RNA:DNA duplex formed at the previous step.

Preparation of double-stranded cDNA was performed by using PCR with the first strand cDNA, obtained above, and the following primers (Retrogen Inc): forward primer, specific to the sequences of ARP gene derived from E. tenella ESTs, CCAGTTTTTGCTTTCTTTTCC, and reverse primer, designed to be complementary to the gene from several genera related to Eimeria, TARAASCCRSMCTGGTRMAGGTACTC, where R=A or G; S=C or G; and M=A or C. A degenerated second primer was chosen because of the lack of sequence information for the C-terminal region of ARP from E. tenella at the time. Taq-polymerase™ (Life Technologies) was used with 2 mM of MgCl₂ and 0.2% BSA. The program performed in PCR Express™ (Hybaid) was the following: a) one cycle consisting of 2 min at 94° C., 45 sec at 50° C., 1 min at 72° C.; b) 20 cycles consisting of 30 sec at 92° C., 30 sec at 53° C. (with ramp 1° C./sec), 60 sec at 72° C. (with time increasing 5 sec/cycle); c) 15 cycles consisting of 30 sec at 92° C., 45 sec at 55° C., 100 sec at 72° C. (with time increase 5 sec/cycle); and, d) one cycle consisting of 10 min at 72° C. followed by hold step at 4° C. First strand cDNA (0.2 uL) in 19.8 μL of the polymerase mixture was reacted in 0.2 ml MμlTI Ultra™ tubes (Sorenson BioScience, Inc.). After the completion of the program, the whole volume was separated at 50 V in 0.8% agarose gel made on 0.5×TAE (Tris-acetate: Acetic Acid, pH 8.5) buffer using RunOne™ cell and power supply unit (EmbiTec). The gel was visualized by staining for 10 min in freshly prepared 0.05% ethidium bromide, briefly washed with distilled water, and viewed on Transilluminator Model M-20E (VWR). One major band was spotted, cut from the gel, and cleaned with Wizard PCR™ kit (Promega) using the manufacturer's procedure. The fragment was eluted from the resin into 50 μL of sterile DDW.

The cDNA was inserted in the pCR2.1 TOPO cloning vector (Invitrogen) in accordance with manufacturer's recommendations. The individual fragment (4 μL in water) was mixed with 1 μL of vector, incubated for 5 min at room temperature, and placed on ice. For transformation procedure, 40 μL of E. coli of either NovaBlue™ competent cells (Novagen), or TransforMax EC 100 Electrocompetent™ cells (Epicentre) were mixed with 1-2 μl of the above mixture, and either electroporated by 1500V, 5 msec in 0.1 cm cuvette (BTX) in Model ECM399 electroporator (BTX) (for EC100 cells), or tranformed by heat-shock procedure (30 sec, at 43° C., for NovaBlue cells). The tranformed mixtures were diluted by 500 μL of SOC medium, and incubated for 1 hr at 37° C. The bacterial suspension was then plated at 100 μL/plate onto pre-heated (37° C.) plates with LB medium containing ampicillin (100 μg/mL). IPTG and X-gal were spread over the plate immediately before plating. White colonies (with an insert) were selected and re-plated onto LB plates with 40 ug/mL kanamycin to obtain single colonies. A single colony from each clone was used for analysis of the insert size by PCR, with primers (M13 forward (−40), and M13 reverse) specific for the vector. The conditions for the PCR were the same as described above, with the exceptions that magnesium concentration was 3 mM and final volume was 10 μL per tube. The fragments were visualized with ethidium bromide, as above. The clones with insert size 0.5-0.6 kB were analyzed by PCR with primers (synthesized by Retrogen Inc.) designed to be specific to the E. tenella ARP on the basis of comparative analysis of available ESTs: forward primer AAATGGGAGAAGCAGACACC, and reverse primer TCCGCGAAGTTAAGAGCTGTTG. The PCR program was the same as above with the exception that the annealing temperature was 56° C. at the second stage, and 58° C. at the third stage. The clones that showed a single band in the agarose gel were chosen for further investigation.

Samples for DC/IL12 assay were prepared by growing selected clones aerobically (shaking for 20 hr at 37° C.) in 3 mL of liquid LB medium supplemented with 40 μg/mL of kanamycin within sterile 15-mL polypropylene centrifuge tubes (Fisher). The bacterial cells were collected into 1.7 mL SafeSeal MμITI™ microcentrifuge tubes (Sorenson BioScience, Inc.) by centrifugation at 10,000×g for 5 min, +4° C., microcentrifuge HSC10K™ (Savant). The supernatant was removed and filter sterilized in Spin-X centrifuge tube with 0.22 μm cellulose acetate filter (Costar) (5 min at 3,000×g). The bacterial cells were washed with 0.5 mL of filter sterilized PBS, re-pelleted at the same conditions as above, and re-suspended in 0.2 mL of sterile PBS. Bacterial cells were disrupted by ultrasound in the Sonic Dismembrator™ Model 100 (Fisher), 3 pulses of 10 sec each at 30 sec intervals; with all procedures performed on ice. The samples were clarified by centrifugation (10,000×g, 10 min, +4° C.) in HSC10K, and the supernatants were filter sterilized in Spin-X tubes as above. For DC assay, 10 μL from each spent medium, and supernatants of disrupted bacteria, was used per each well of the assay.

The recombinant form of ARP, generated in E. coli from the E. tenella cDNA, have approximately the same specific activity as purified bovine molecule and ARP extracted from E. tenella oocyts. See FIG. 5. 50% activity occurs at approximately 50 femtomolar active protein concentration. The recombinant material was obtained from the cloning vector, pCR2.1 TOPO. The vector allows for expression from cDNA placed in frame to LacZ gene. The whole construction as ARP protein fused to β-galactosidase was obtained from the supernatant of a Liter of minimal growth media. The supernatant was purified using DEAE chromatography, with the method described in section 6, and was separated by C8 HPLC, using the method of section 6. One of two active peaks, containing approximately 200 ng of protein, was assayed on the DC assay to yield the response in FIG. 6.

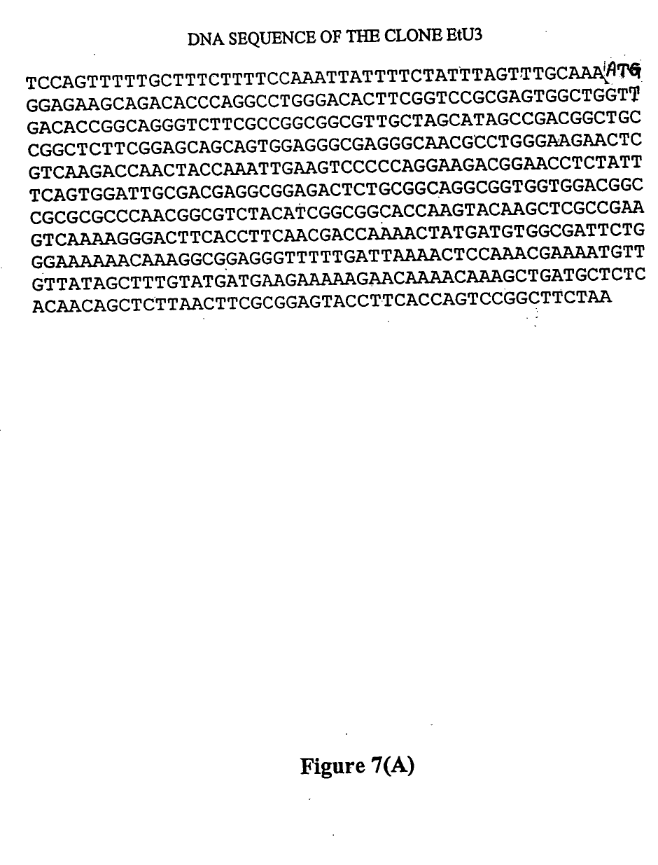

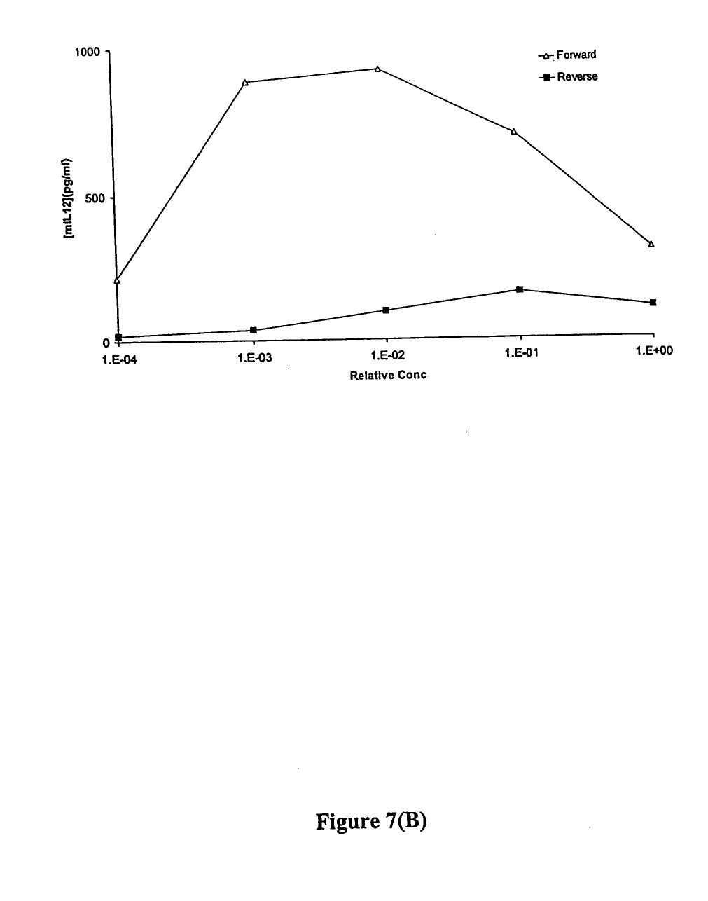

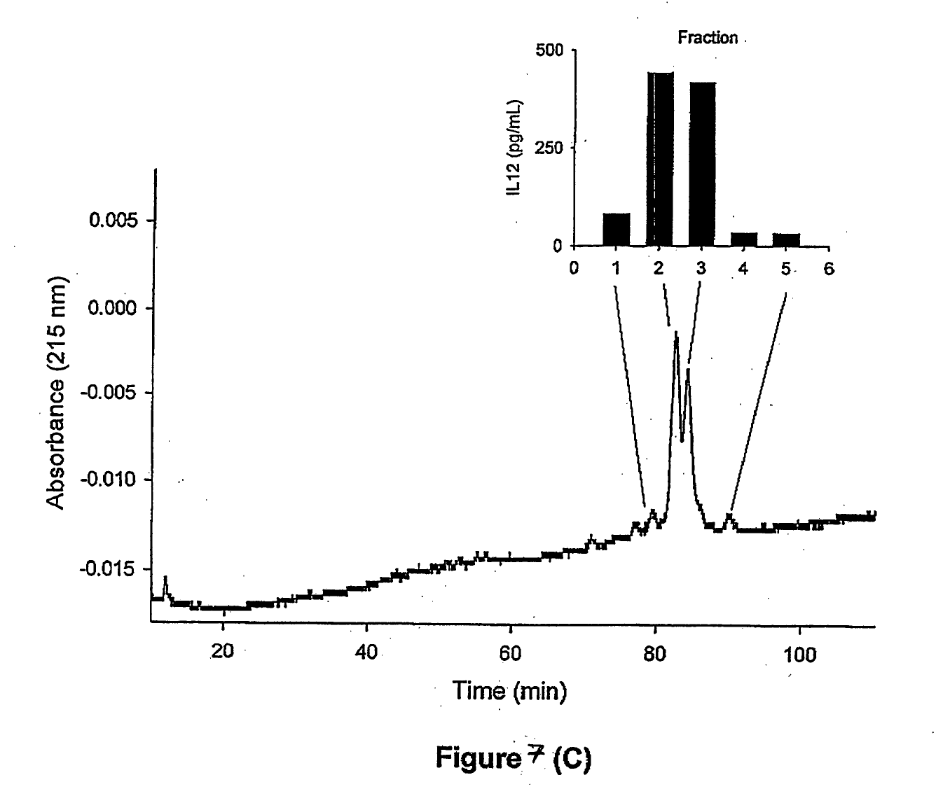

The cDNA for E. tenella ARP was inserted in the pCR2.1 TOPO cloning vector (Invitrogen). Appropriate clones were selected by their insert size, sequence, and orientation. While not an optimized expression vector, some protein was produced from the insert in a form of fusion protein and exhibited DC activity. The DNA sequence of the insert, denoted EtU3, is given in FIG. 7A. These inserts are being transferred to expression vectors for E. coli and eukaryotic cells. The expressed protein will contain a removable tag sequence to facilitate identification and purification. Activities of the products from two E. coli cultures containing the cloning vector, with the insert introduced in either direct or reverse orientation to LacZ promoter, are shown in FIG. 7B. Only the correctly oriented ARP gene exhibited DC activity. This demonstrates proof that the activity is associated with this molecule because everything else between the two cultures is identical.

C8 chromatographic purification of the protein produced by the cloning vector in E. coli, growing in minimal mineral media, is shown in FIG. 7C, with DC activity included in the inserted graph. The activity is weak but is located at the similar retention time as the activity extracted from the sporozoites. The E. coli media was first purified by DEAE chromatography followed by Superdex™ chromatography yielding a highly pure product. The purity is shown as two active peaks on the analytical C8 chromatography column (FIG. 7C). The data suggest that N-terminal region can be extended significantly without any loss in DC-activity. The ARP molecule, fused at the N-terminus, to either a portion of β-Galactosidase, maltose binding protein (MalE), FLAG peptide, or cellulose binding protein (CenA), retained significant activity. Point mutations at two positions nearby the C-terminal end (Y to H, and G to either A, or S) apparently did not change the DC-activity of the mutant as well. To reveal the importance of the C-terminus for activity of the ARP molecule, a truncated ARP molecule was created by introduction of a termination codon 14 amino acids prior to C-terminus by PCR (primer: CTATGTGAGAGCATCAGCTTTG). It showed significantly lower (several orders of magnitude lower) DC-activity compared to full-length control. Both were expressed from T7 promoter with Shine-Dalgarno ribosomal binding site (RBS) introduced in front of the ATG start codon by PCR (primer: GAAGGAGTTTGCAAAATGGGAA). Therefore, the missing sequence (TALNFAEYLHQSGF) is important for either structure or activity of ARP.

Transferring the cDNA to an expression vector (pMAL-pZE, New England Biolabs) have successfully generated clones with DC activity. This vector contains the malE gene (for fusion to maltose binding protein at N-terminus) adjacent to enterokinase cleavage site, which allows rapid separation and purification of the expressed protein. The fused ARP protein was expressed by IPTG induction, and appeared to be active as checked by DC assay. Release of the ARP protein from the fusion construction by cleavage with enterokinase did not result in enhancement in DC activity, arguing that the foreign part of the recombinant molecule may not impact the activity.

For stable expression of ARP in mammalian cells, the E. tenella ARP gene was re-cloned into two vectors: pTracer-CMV2 (Invitrogen) or p3XFLAG-CMV9 (Sigma). Uni-directional cloning into vector pTracer-CMV2 was performed into KpnI and XbaI sites. The fragment for cloning was created by PCR amplification of the gene with primers bearing newly created sites Kpn I (in front of start codon) and Xba I (after the stop codon). The ARP gene missing the start codon and cut out of the construction in pMal-p2e vector flanked by Hind III sites used for cloning into a Hind III site of the vector p3xFLAG-CMV9. Orientation and in-frame position of the gene relative to secretion signal peptide as well as 3×FLAG peptide were confirmed by complete sequencing of the insert. Both plasmids were used to transform CHO-KI and S-180 cell lines with Lipofectamine 2000 (Invitrogen) according to manufacturer recommendations. Individual clones of transformed cells were obtained after selection in growth media containing antibiotic (Zeocine for pTracer-CMV2, and G-418 for p3XFLAG-CMV9), followed by re-seeding at low cell density into 96-well plate. In both cases the spent media showed high activity on DC-assay (up to 103 dilution) implying that ARP is secreted out of the cells. Analysis of the media by C8 RP-chromatography revealed the presence of a single peak of DC-activity eluting in the position close to that of bacterially expressed ARP. Preliminary calculations showed that specific activity of ARP expressed in mammalian cells is comparable to or higher than that of bacterially expressed protein.

### 11.2 Isolation of E. Tenella ARP from E1 Clone

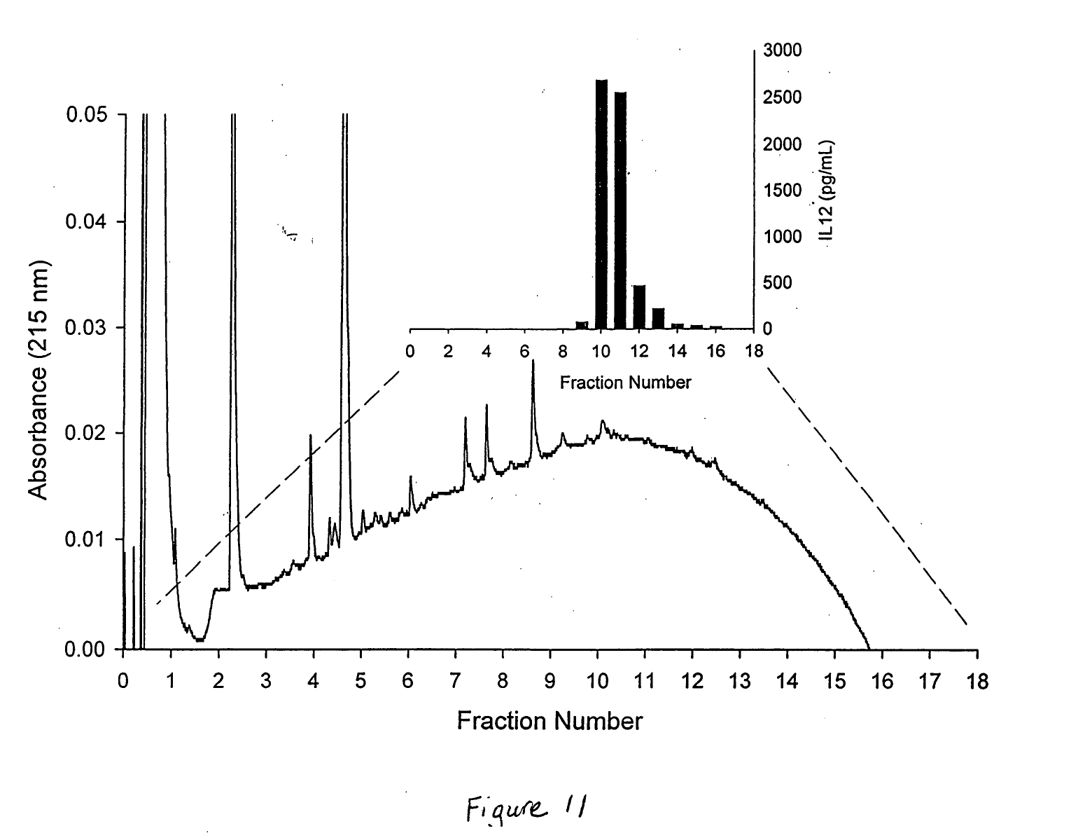

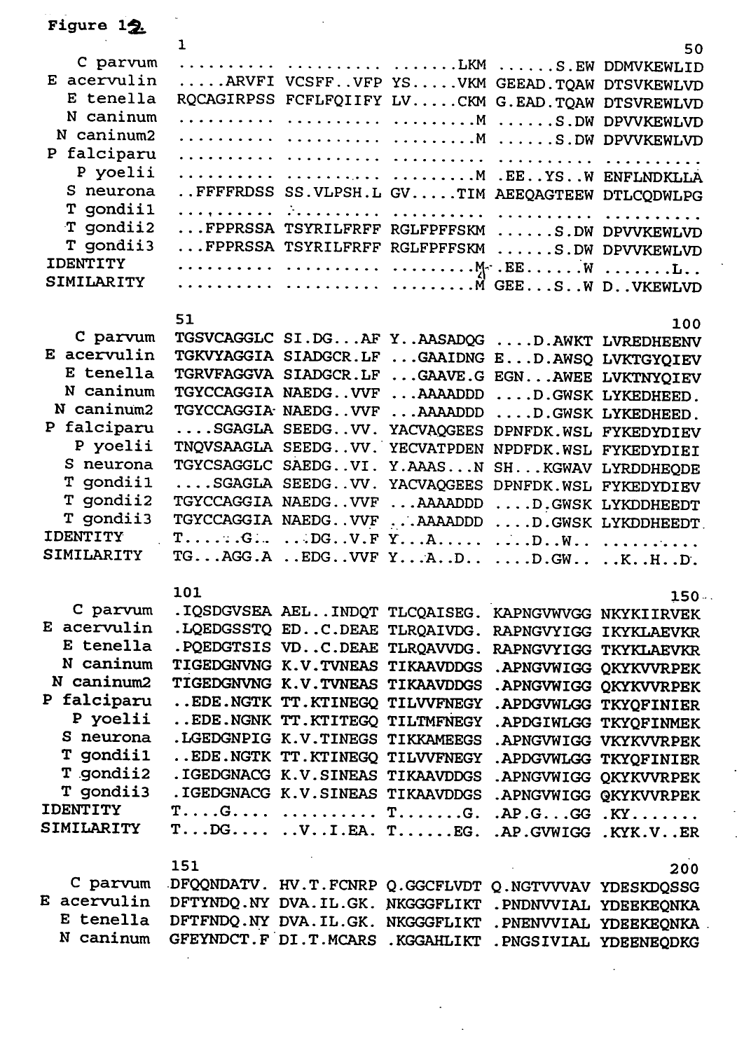

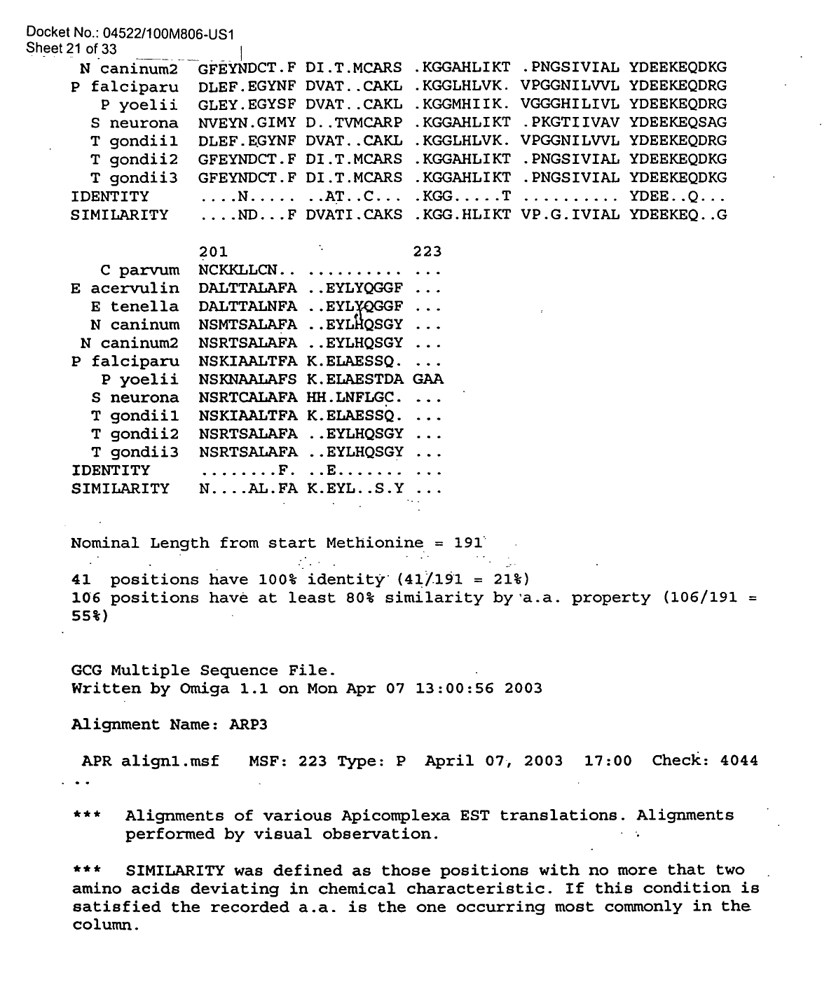

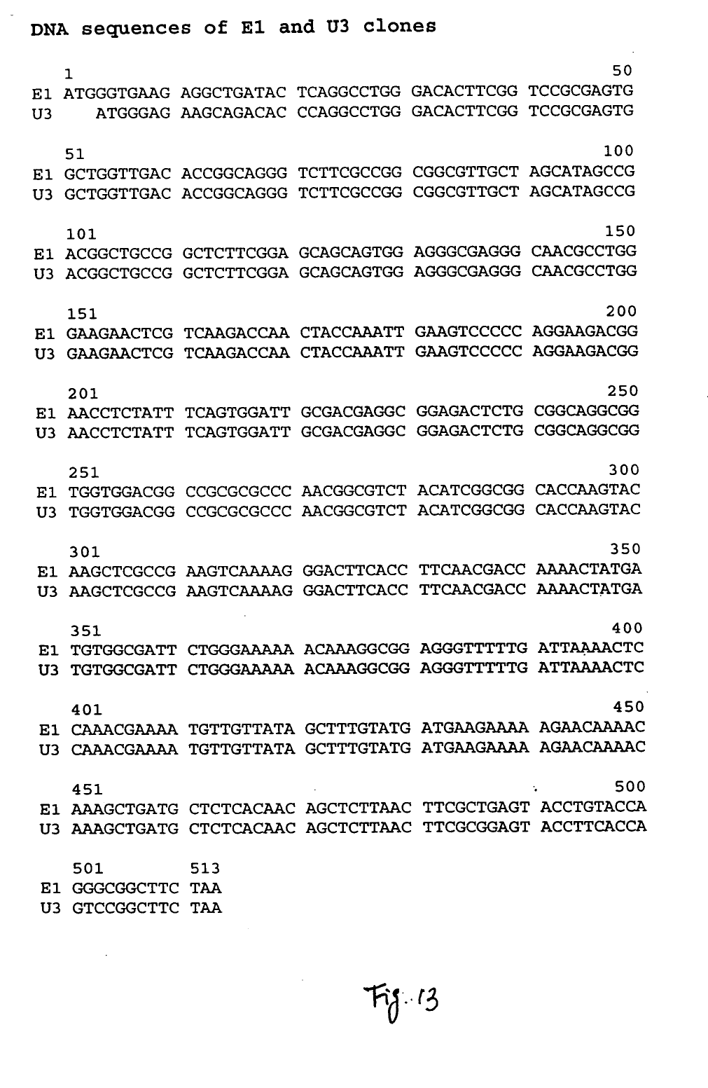

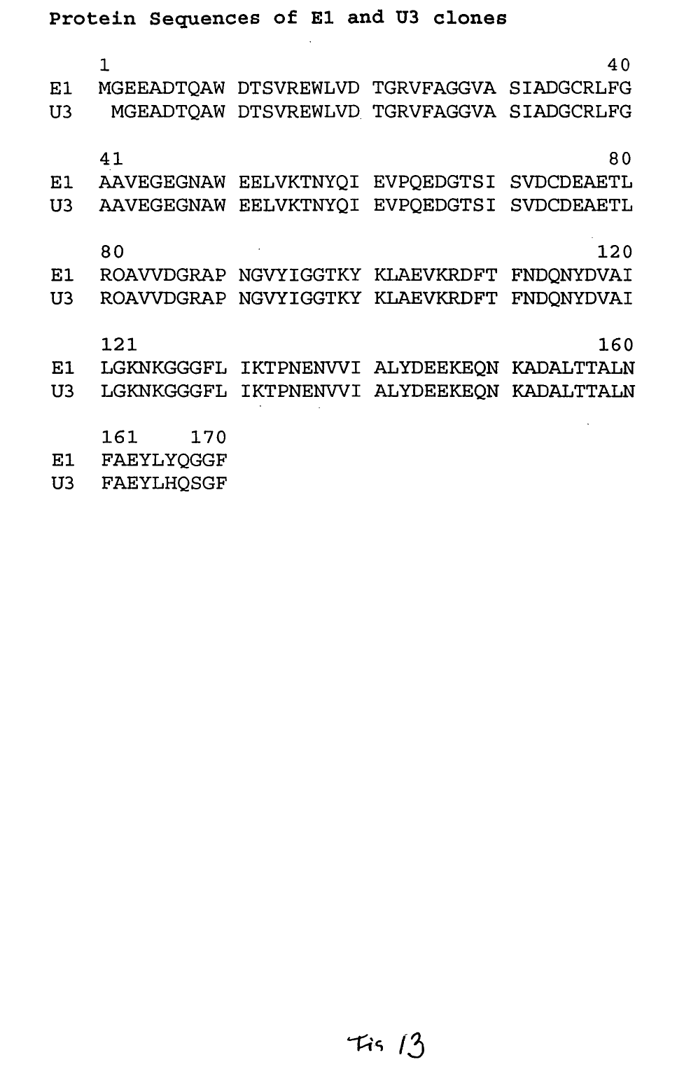

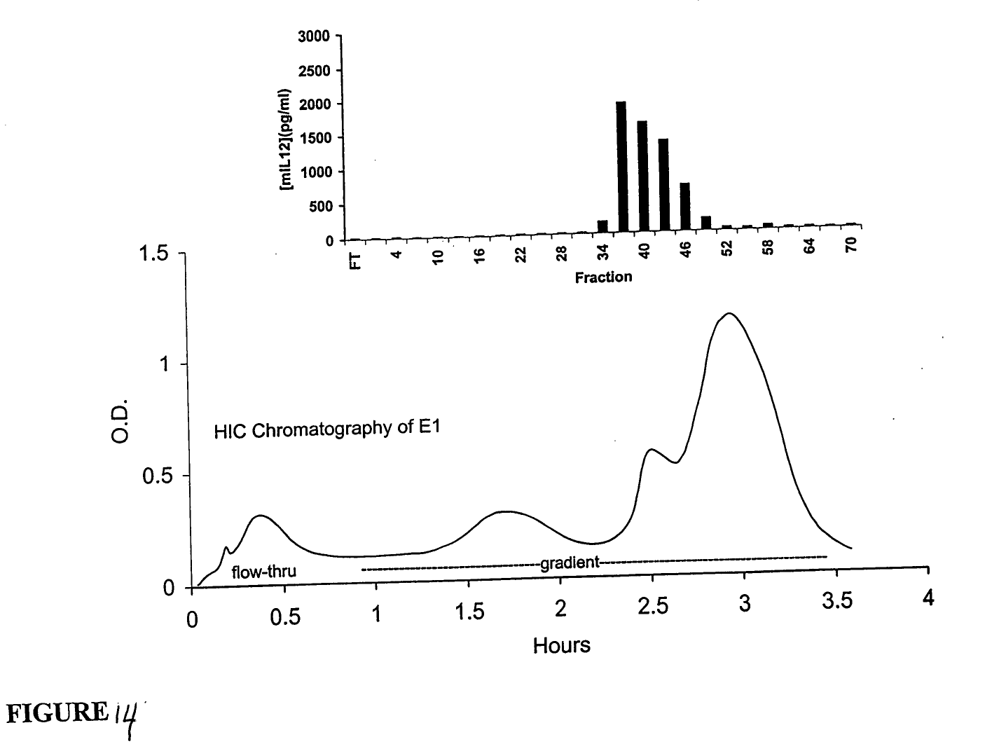

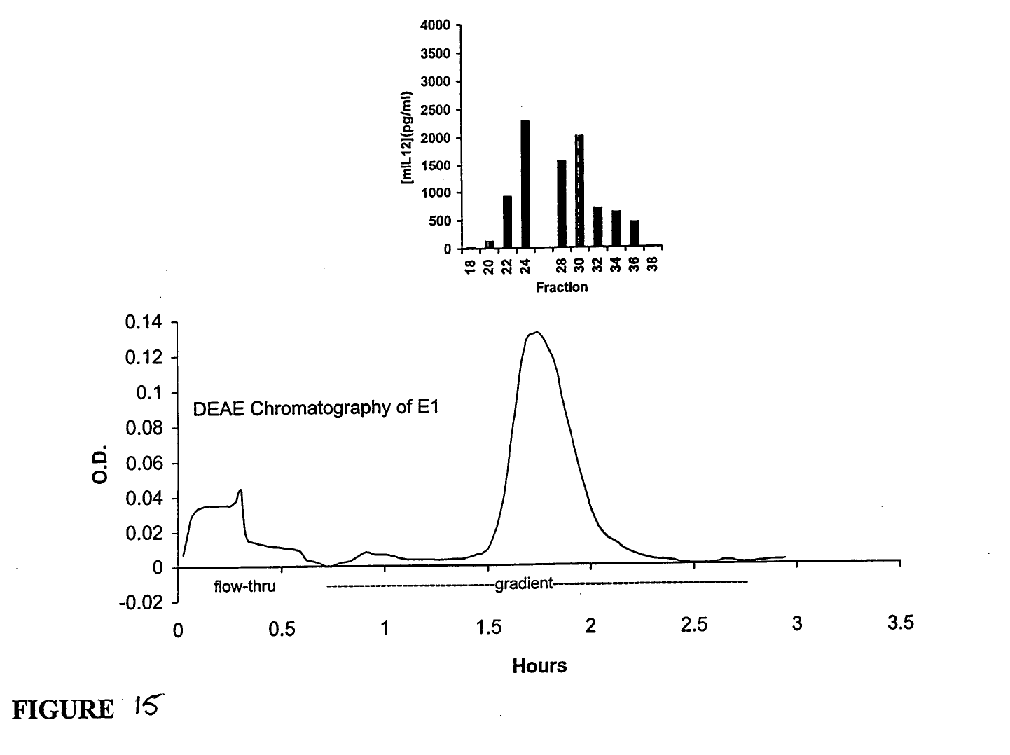

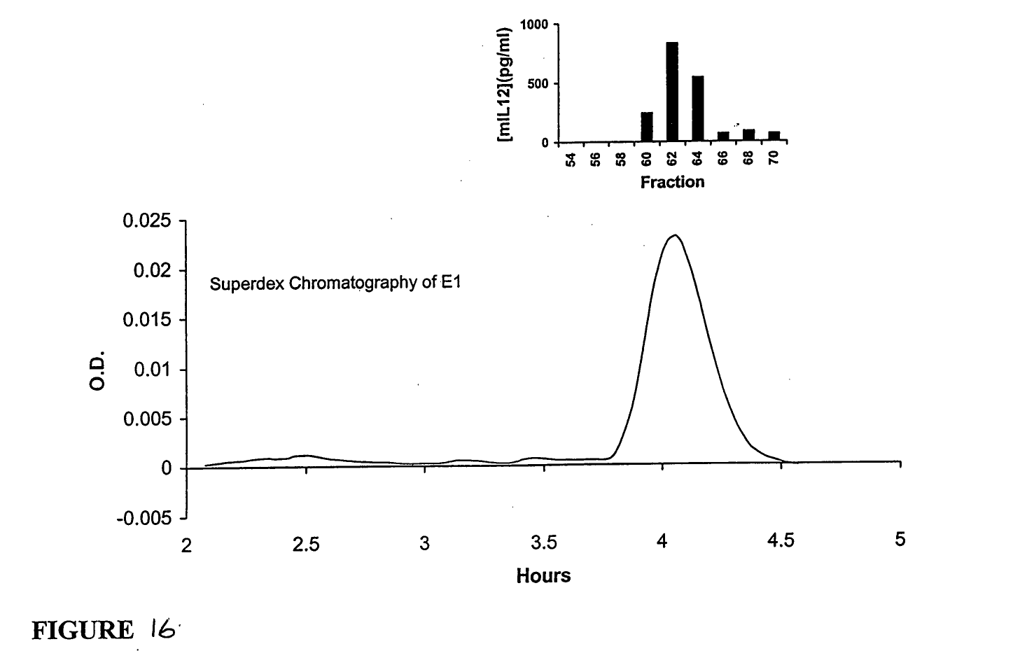

The E. tenella gene coding for ARP was cloned by PCR using forward primer (ATGGGTGAAGAGGCTGATACTCAGG) and reverse primer (TTAGAAGCCGCCCTGGTACAGGTACTCAGCGA). The PCR fragment was ligated to the pET-Blue-1 acceptor vector (Novagen) with clonableRT (Novagen) overnight at 16° C. The resulting plasmid was transformed into E. coli host Nova Blue with carbenicillin/tetracycline and white/blue clone selection. Positive clones were identified by colony PCR using the same forward primer and pET Blue Down primer #70603-3 (Novagen) to assure DNA insert of correct orientation. The DNA sequence of the insert was confirmed by sequencing. The plasmid of the positive clone was isolated and transformed into host strain Turner™ (DE3)pLacI (Novagen) for protein expression. One stable clone was designated as E1. (See FIG. 13). Clone E1 was deposited with the American Type Culture Collection on Jul. 15, 2004 and assigned patent deposit number ______.

Single clone of E1 from agar culture was inoculated in 10 ml LB medium supplemented with 1% glucose in the presence of 50 μg/ml carbenicillin and 34 μg/ml chloramphenicol. The culture was maintained overnight at 37° C. with shaking at 250 rpm. The culture was then added to 1 liter of the same medium and allowed to grow for 2.5 hours to absorbance of about 0.6-0.8 at 600 nm. Protein expression was induced by addition of isopropyl-1-thio-β-D-galactospyranoside (IPTG) to 0.5 mM final concentration. After 4 hours, the bacteria were harvested by centrifugation at 8,000 rpm (6,500×g), for 20 min. The supernatant was removed and saved and the pellet was frozen until used.

The bacterial pellet was resuspended in 30 ml PBS and passed through a French press three times at 1,600 psi to disrupt the cells. The press cell was rinsed with PBS to recover residual cell sample. The sample was centrifuged at 15,000 rpm (17,500×g) with SS34 rotor for 30 min to remove cell debris. The supernatant and the pellet were separately saved. Total volume of the supernatant totaled to about 38 ml. Ammonium sulfate was then added to the supernatant to 30% saturation. After 1 hour at 4° C. with stirring, the precipitate was removed by centrifugation. The ammonium sulfate was further added to 45% saturation. After 1 hour, the precipitate was removed. Ammonium sulfate was added to 80% saturation. The sample was stirred overnight at 4° C. The precipitate was recovered by centrifugation. The pellet was resuspended in 1.5 M ammonium sulfate, 50 mM sodium phosphate, pH 7.0. The sample was then centrifuged to remove undisolved material.

Hydrophobic Chromatography on Phenyl-Sepharose™ Column:

The soluble fraction was subjected to hydrophobic chromatography on a phenyl-Sepharose column (Fast Flow, (low sub), by Pharmacia 3×3.3 cm). After the sample was applied to the column, it was washed with the same buffer until the baseline was obtained. A reverse linear gradient of 1.5 M to 0 M ammonium sulfate, 50 mM Na phophate, pH 7.0 (200 ml for each buffer chamber) was applied at room temperature at a flow rate of 2 ml/min. 5 ml fractions were collected. Selected fractions were analyzed by DC assay and SDS-PAGE. The active fractions were pooled and dialyzed overnight at 4° C. against 0.2 M NaCl, 10 mM Na phosphate, pH7.0 to be ready for the DEAE-Sepharose™ chromatography.

DEAE-Sepharose™ Chromatography:

The DEAE-Sepharose™ column (Fast Flow, by Pharmacia; 1.5×5.5 cm) was equilibrated with the initial buffer (0.2 M NaCl, 10 mM Na phosphate, pH 7.0). After the sample was applied, the column was washed with the same buffer to remove unbound material as monitored by Abs280. When the absorbance came to the baseline, a linear gradient (0.2 M-0.6 M NaCl in 10 mM Na phosphate, pH 7.0, 200 ml for each buffer) was applied. Fractions of 5 ml were collected. The eluted fractions were analyzed by DC assay, and SDS-PAGE.

Superdex™ Size Exclusion Chromatography

Final purification and elimination of endotoxin was accomplished by Superdex™ 75 chromatography using the method described in Section 6. Active samples were stored at 4° C. instead of freezing.

C8 Chromatography for Quality Control

Reverse phase HPLC (as described in Section 9) was performed on a sample of the pooled active material from the Superdex chromatography.

### 12. EXAMPLE 7

### Physical Properties of ARP

During the many years of dealing with an impure preparation of ARP, it was difficult to obtain information on the physical characteristics of the molecule. Initially, for example, it was not obvious if it was a protein or another type of macromolecule. In retrospect, it is clear that the extremely potent nature of the protein meant that it was always a very minor component in any partially pure preparation. Some findings related to the nature of ARP molecule are contained in the following subsections.

Molecular Weight

Several experiments have indicated that the molecular weight range of ARP is centered near 20 kD. A large amount of indirect evidence for this molecular weight has come from gel banding in SDS-PAGE for known active fractions. Similarly, activity has routinely eluted from sieving chromatography columns in this range. Direct evidence has been obtained from large SDS-PAGE slab gels. Active material has been applied to this system, with a slight modification in sample preparation to avoid boiling in SDS, and after electrophoresis, the gel was sliced and the proteins were eluted, renatured and tested. The only recoverable activity was located in the 20 kD (18±3 kD) range.

This has been validated with final purification and sequencing. The homologous protein from avian Eimeria is reported to have an 18.5 kD predicted mass and antibodies to a peptide segment react with a 20 kD band from E. tenella and E. acervulina (Lillihoj et al., 2000).

pI Determination

Several experiments on extracting and re-folding proteins from an acrylamide gel after isoelectric focusing revealed a pI being in range of 4.0-4.7 for protein active in NK assay, while the predicted pI for E. acervulina, and E. tenella ARP proteins is 4.3-4.5. These results are consistent with acidic protein containing a significant number of carboxyl groups.

Isoelectric focusing was performed with the use of a precast pH 3-7 IEF gel (Novex) according to company instructions. Broad pI calibration kit (Pharmacia) was used as a standard for pI calculation.

pH Dependence of ARP Activity

Several experiments revealed significant dependence of ARP activity on pH. In crude materials, NK activity was found to decline significantly at both high pH>10, and low pH<4. In some experiments, minimal activity was observed below pH4.0, while one experiment showed minimal activity in the range 3.4<pH<3.7. While the loss of activity at high pH can be at least partially reversed by re-naturation process, the impact of low pH in many cases (specially at pH<2.0) was irreversible.

Temperature Stability

The stability of drug product BBX-01 was evaluated at several temperatures to help determine shelf life and storage conditions. The results are shown in Table 15.

TABLE 15
Estimates of BBX-01 Thermo-stability

| | Time to 50% loss | | Comments |
|---|---|---|---|
| | (estimated from presumed | | (activity measured |
| Condition | exponential loss) | | with NK Assay) |
| −80° C. | >>1 | yr | No detectable loss |
| | | | in 24 months |
| 4° C. | >18 | months | No detectable loss |
| | | | in 18 months |
| Ambient (˜24° C.) | ˜6 | weeks | ˜20% loss at two |
| | | | weeks |
| 37° C. | ˜6 | days | |
| 56° C. | ˜11 | min | |

| | |
|---|---|
| 16 Freeze-thaw cycles | No detectable loss |
| (liquid N₂ to ambient) | of activity |

Activity was undiminished for extended periods in frozen and refrigerated conditions, while it decreased as the temperature increased. Even at 37° C., however, the half-life was nearly one week, suggesting that the usefulness as a drug would not be limited to auto-degradation at body temperature.

Stability of E1 in Human Serum Albumin

Preliminary studies on the stability of the recombinant ARP, E1 (1 μg/mL) in the presence of HSA (Human Serum Albumin) at 1, 5, or 10% w/v indicate no loss in activity at 4, 11, 25, 39 and 54 days when stored at 4° C. Higher temperature studies also indicate remarkable stability. No loss of activity was detected after 41 days at 37° C., 6 hours at either 55° C., or 2 hours 70° C. while in the presence of HSA. The increase in stability over BBX-01 is probably due to the higher purity of the E1 preparation, which avoids possible protease contaminants. For use in human clinical trials. E1 has been formulated at 1 μg/ml with 3% HAS and stored at 4° C. for up to 29 weeks without loss of activity as measured by the DC assay.

Enzyme Sensitivities of ARP

There was compelling evidence that the partially purified material contained a protein and not a nucleic acid, glycolipid, lipopolysaccharide, or some complex of these with a peptide component. Treatment of the partially purified fraction with the proteases trypsin, chymotrypsin, V8, subtilisin, and papain were all successful in eliminating activity. Careful titration experiments indicated that, on average, a single cut from the protease subtilisin was sufficient to eliminate all activity, while chymotrypsin required three cuts to reduce activity 10-fold. These results gave strong evidence that a major component of the active molecule was protein. Additional catabolic enzymes were tested to test the possibility that a modified protein or complex was important to activity.

In several experiments with the nucleases, e.g., RNase A and DNase I, the activity of ARP was followed in the NK and IFNγ assays. For both enzymes, there was no loss of activity after 9 and 24 hours of incubation at 37° C. as compared to ARP incubated in the same conditions without the enzyme. In neither case, migration of the NK-active component in 4% agarose gel was affected by the treatment with the enzymes, while the visible nucleic acid bands disappeared. This indicated that no nucleo-protein complex was important to the activity.

Several experiments were also performed with deglycosylases on deglycosylation of crude bovine protein preparations with peptide-N-Glycosidase F from Flavobacterium meningosepticum, specific for N-linked carbohydrate moieties. These showed that the position of the active molecule did not move on polyacrylamide gels after digestion. Activity, however, was reduced by approximately 40% after incubation with the enzyme. This provides evidence for a short carbohydrate chain that may provide stability to the molecule or its removal reduces the activity due to introduction of an extra negative charge (Asn>Asp). Two potential N-glycosylation sites (Asn-Gly-Thr) are present in the bovine ARP sequence (see Table 4).

Effect of Divalent Cations on DC-Activity and Aggregation State of ARP

Salts of alkaline earth metals (CaCl₂, MgCl₂) and transitional metals (ZnCl₂ and CuCl₂) were tested for inhibition of DC-activity induced by semi-purified ARP preparations from bovine intestine and recombinant ARP. With crude ARP preparations, ZnCl₂ and CuCl₂ consistently showed inhibition of activity in mM-μM concentration range (which is in physiological range), while CaCl₂ and MgCl₂ did not. Some differences exist in the interaction of copper and zinc with ARP and must be explored further. Both transitional metals also inhibited the DC-activity of purified recombinant ARP. However, preliminary experiments involving pre-incubation with 10 mM salts of alkaline earth metals on recombinant ARP showed different effects in ARP preparations from bacteria grown on either mineral media (M9 with 2 mM MgSO₄, 0.1 mM CaCl₂, and no trace metals) or rich (LB) media. The former was inhibited by both-calcium and magnesium ions, while the latter—only by magnesium ions. The difference can be due to different local concentrations of divalent metals or other co-factors during synthesis and folding, producing different sets of conformational isomers of ARP.

Experiments on the mechanism of activity inhibition by Zn²⁺ revealed that aggregation of ARP occurred in the presence of 10 mM ZnCl₂ yielding high molecular weight structures pelletable by centrifugation at 15000 g for 30 min. The data suggest that binding of divalent cations induces a change in the tertiary structure of ARP, exposing inner hydrophobic regions that result in aggregation and/or interaction with other proteins and this leads to a loss of activity.

Inhibition by representatives of both metal groups can have an impact on design of ARP treatment and purification protocols. The alkaline earth metals are abundant in all tissues and organs. The physiological concentration of Zn²⁺ varies from 10 μM in serum to 3 mM in seminal fluid. Concentration of Zn²⁺ and Cu²⁺ was shown to change during some diseases including malignancies. In some tissues and/or pathological conditions the concentration of these metals may be high enough to interfere with anti-tumor activity of ARP. In these cases the local administration of metal-chelators might have to be included in ARP treatment protocols.

### 13. EXAMPLE 8

### Measurement of ARP Activities

### 13.1 NK Assay

Materials

DMEM/F12 culture media, fetal calf serum (FCS), and human recombinant interleukin 2 (hr-IL2) (R & D Systems, Inc) were obtained from Gibco/BRL. Percoll™ (Pharmacia, Inc), gentamicin and MTT were obtained from Sigma Chemical Co. Mouse epidermal growth factor (EGF), bovine insulin and human transferrin were purchased from Collaborative Research Corp. Isolation of small intestinal extracts (MEX-SI) and isolation of spleen-derived NK cells were purchased from Harlan Bioproducts for Science (Indianapolis, Ind.).

NK (LGL) Cell Isolation

Spleens (10-15 per experiment) were aseptically removed from male Balb/C mice 6-10 weeks of age. Splenocytes were “squeezed” out of spleens using 2 sterile glass microscope slides. Cells were collected in approximately 10 ml of DMEM/F12 containing 10% fetal calf serum (FCS) and gentamicin (50 μg/ml). Single cell suspensions were generated by passing collected cells through a 70 μm nylon mesh screen. After washing the cell pellet once with PBS (centrifuged at 675×g for 5 min), red blood cells were lysed by brief hypotonic shock (i.e. exposure to sterile distilled, deionized water, followed immediately with appropriate volumes of 10×PBS to return to isotonicity). Remaining cells were centrifuged and resuspended in DMEM/F12+10% FCS and transferred to 75 cm² flasks each containing 25 mls DMEM/F12+10% FCS (cells from 5 spleens per flask). The cells were incubated for 60 min. at 37° C. to selectively remove readily adherent cells (mainly fibroblasts and macrophages). After the incubation, the flasks were gently shaken and the non-adherent cells were removed, pelleted by centrifugation, and resuspended in DMEM/F12 (no supplements) (1.0 mL/10⁸ cells). A 1.0 mL volume of these cell suspensions were carefully layered onto a 70%/60%/40% (2 mL/4 mL/4 mL) Percoll™ gradients in 15 ml centrifuge tubes and centrifuged for 30 min at 675×G. The cells which sediment at the 40/60 interface represent primarily large granular lymphocytes (LGLs), enriched with NK cells. These cells were carefully removed with a pasteur pipet, centrifuged, washed once with PBS and resuspended in approximately 5 mL DMEM/F12 supplemented with gentamicin (50 μg/mL) and 10% fetal calf serum. Cell counts and appropriate dilutions of LGL cells were made with these cells, as described below. For convenience this LGL cell preparation is interchangeably referred to as NK cells.

NK Cell-Mediated Cytotoxicity (NK-CMC) Assay

Mouse sarcoma 180 cells were seeded into 96 well plates at a density of 5×10³ cells per well in 100 μl DMEM/F12, supplemented with 10% FCS and gentamicin (50 μg/mL). After several hours to ensure proper attachment, test samples (e.g., samples containg an ARP protein, positive or negative controls) were added to each well in volumes of 10-25 μL. NK cells were added in 100 μL of supplemented DMEM/F12 at designated densities: 0 NK cells/well; 5×10⁴ NK cells/well (1:10 target/effector ratio); 1.25×10⁵ NK cells/well (1:25 target/effector ratio); 2.5×10⁵ NK cells/well (1:50 target/effector ratio); and, 5×10⁵ NK cells/well (1:100 target/effector ratio). (Note: routinely, only the 1:50 and 1:100 ratios were used for screening purposes. IL2 was added as a media supplement with final concentration of 125 U/mL.). Co-cultures were incubated for 4 days at 37° C., 5% CO₂, and terminated and vitally stained using a MTT cell viability quantification assay (see Mossman, T. J. Immunol. Methods 65:55 (1983); and Sigma Chemical MTT (M5655) product application note). Culture media was carefully aspirated from each well and the remaining cells were washed twice and replaced with 100 μL DMEM/F12+10% FCS, containing MTF (50 μg/mL). The plates were further incubated for 4-5 hours at 37° C. Absorbance was measured with the aid of a plate reader (600 nm filter). Decreased absorbance indicated a decrease in the number of viable cells per well (i.e. cytotoxicity). Absorbance was measured again after the MTT was solubilized by replacement of the medium with 200 μL of 2-propanol containing 0.04M HCl. This gave a uniform color throughout the well and minimized discrepancies in absorbance readings due to uneven cell distribution. NK-inducing activity was calculated relative to negative (PBS/BSA) and positive (internal standard) controls.

### 13.2 Dendritic Cell (DC) Assay

To follow the activity in semi-purified preparations of BEX, an assay which follows IL12 release from freshly isolated dendritic cells (DCs) as an index of DC activation was developed.

Special Reagents

Mouse recombinant GM-CSF, IL4 and IFNγ and anti-mouse CD40 were obtained from R&D Systems (Minneapolis, Minn.). Collagenase D was obtained from Boehringer Mannheim (Indianapolis, Ind.). MiniMACS magnetic cell isolation system was obtained from Miltenyi Biotech (Auburn, Calif.).

Isolation and Culture of DCs

DCs were isolated from the spleens of male BALB/c mice 8-12 weeks of age (Harlan Bioproducts for Science, Indianapolis, Ind.), using the magnetic assisted cell separation (MACS) system purchased from Miltenyi Biotech. In summary: Spleens were aseptically removed from mice and placed into a petri dish containing 5 mL holding media (DMEM/F12 supplemented with 10% FCS and antibiotics). Each spleen was then injected with ˜0.5 mls collagenase D solution (1 mg/mL in 10 mM Hepes-NaOH, pH 7.4, 150 mM NaCl, 5 mM MgCl₂, 1.8 mM CaCl₂), placed into 5-10 mL collagenase D, cut into 5-6 pieces and incubated at 37° C. for 45-60 min. Collagenase-digested spleens were then processed with glass slides and passed through a 70 μm nylon mesh cell strainer. After the strainer was flushed with 40 mL culture media, cells were centrifuged (8 min @ 200×G), suspended in 1 mL PBS and subjected to red blood cell lysis by incubating the cell suspension for 5 min at room temperature in 10 mL lysis buffer (155 mM NH₄Cl, 10 mM KHCO₃, 0.1 mM EDTA), followed by dilution in PBS to 50 mL and centrifugation (8 min @ 200×g). The splenocytes were then suspended in 10 mL MACS buffer (PBS pH 7.2, supplemented with 2 mM EDTA and 0.5% BSA), passed through a 40 μm nylon mesh cell strainer which was flushed with 40 mL MACS buffer. A preliminary cell count was taken at this time, prior to centrifugation (8 min @ 200×G). The splenocytes were suspended at a density of 10⁸ cells per 400 μL MACS buffer. Paramagnetic MicroBeads coated with anti-CD11c antibody were mixed with the splenocyte suspension (100 μL per 10⁸ cells), incubated at +4° C. for 15 min, washed, suspended in 1.0 mL MACS buffer and passed through a MS+separation column placed within a strong magnetic field. CD11c⁺ cells were retained in the column whereas CD11c⁻ cells were washed off the column as the flow-through cell fraction. CD11c⁺ cells were then collected by removing the column out of the magnetic field and flushing the column twice with 1 mL of MACS buffer. CD11c⁺ cells were washed once with culture media and suspended at a density of 0.5×10⁶ cells per mL in cytokine supplemented culture media {DMEM/F12 supplemented with 10% FCS and gentamicin containing mouse GM-CSF (1 ng/mL), mouse IL-4 (1 ng/mL), mouse IFNγ (3 ng/mL) and anti mouse CD40 (0.5 mg/mL)}. CD11c⁺ cells were then added (100 μL/well) to 96-well tissue culture plates containing test samples (e.g., samples containing an ARP protein, positive or negative controls) diluted in 100 μL media per well and cultured overnight at 37° C. in 5% CO₂, 95% air.

Determination of Mouse IL12 (p70% Release from DCs

Mouse IL12 release from CD11c⁺ splenocytes was measured using ELISA. Briefly, CD11c+ cell culture supernatants were sampled following an overnight incubation (usually 15-18 hrs). Media samples (100 μL) were added to ELISA plates coated with anti mouse IL12 (p70) capture antibody (R&D #MAB419; 250 ng in 100 μL per well) and incubated either at 37° C. or room temperature for 2 hrs. The ELISA plates were washed extensively with wash buffer after which 100 μL per well of detection antibody/detection reagent (biotinylated anti-mouse IL12; R&D #BAF419 at 50 ng/mL and streptavidin-HRP) was added. The ELISA plates were again incubated for 2 hours, washed and exposed to TMP substrate solution (Pierce #34021) for 20 min. The substrate reaction was stopped by adding 100 μL/well 2M H₂SO₄. ELISA plates were read at 450 nm (corrected at 540 nm) and mIL12(p70) levels were calculated using an intra-ELISA standard curve (range: 2000-31.25 pg/mL).

### 13.3 IFN-γ Assay

The production and release of IFNγ by LGLs into the culture media was assessed as follows: LGLs, enriched with NK cells, were isolated and seeded into 96 well plates at densities of 2.5 or 5.0×10⁵ cells/well, in 200 uL of DMEM/F12 supplemented with 10% fetal calf serium, gentamycin (50 ug/mL), and IL2 (125 U/mL). Test samples (e.g., samples containg an ARP protein, positive or negative controls) were added and the cells were cultured overnight, following which the condition media (CM) was removed and centrifuged to remove any aspirated cells. Aliquots of the CM were then measured for IFNγ using an ELISA kit purchased from various sources (BD PharMingen Inc., Genzyme Corp., and R&D Systems Inc.). Briefly, in the ELISA assay, CM or serum samples (and IFNγ standard) are incubated in ELISA plate wells, previously coated with a capture antibody (hamster monoclonal anti-mouse IFNγ), for 1 hr at 37° C. After extensive washing the wells are exposed to a biotinylated second antibody (polyclonal anti-mouse IFNγ) for 1 hr at 37° C. After washing, the wells are then exposed to a detection reagent (streptavidin conjugated with horseradish peroxidase) for 15-20 min. at 37° C. Once again, after extensive washing, 100 μL TMB substrate is added to the wells and incubated for 5-7 min. at room temperature. The reaction is stopped by adding 100 μL 2M H₂SO₄. Absorbance at 450 nm is read using a plate reader and IFNγ concentrations are calculated from the standard curve.

### 13.4 Immuno-Stimulatory Activities of ARP

NK Activation

Early in this project it was discovered that cytotoxic lymphocytes isolated from mouse spleens were central mediators of the cytotoxic effects of mouse and bovine small intestinal extracts against S180 cells in vitro. Therefore, a novel in vitro assay to assess induced cytotoxic activity of NK-like cells was formulated and implemented using large granular lymphocytes (LGLs) isolated from mouse spleens using discontinuous Percoll™ gradients. The natural killer (NK) cells are routinely referred as the primarily mediators of the cytolytic effects of SIE in vitro and in vivo. It should be noted, however, that other lymphocytes contained in the LGLs population of spleenocyte may also be involved, such as lymphocyte activated killer cells (LAKs).

The LGLs were co-cultured with S180 tumor cells, at various effector to target (E:T) cell ratios, for 4 days. This protocol is not typically used in other reported experiments on NK activity. It is unique because tumor cells are dividing while the NK activation occurs. This creates a more complex environment that may mimic the in vivo conditions more closely. The culture medium contained a low concentration of IL2 (125 U/ml) plus any experimental (purification) preparation of intestinal extract to be tested. The “basal” activity induced by IL2 alone, at the highest NK:S180 ratio (100_NK), was normally low (10-15%), suggesting that S180 cells are relatively resistant to the cytolytic action of the LGLs cells induced by IL2. However, in the presence of partially purified bovine small intestinal extracts, the cytolytic activity of these LGLs was dramatically and synergistically increased, often resulting in nearly 100% cytotoxicity at effector: target ratios as low as 50:1. (It should be noted that the effector: target ratios are determined at the time of seeding the cells—Day 0. Since the S180 cells undergo 3-4 population doublings during the subsequent culture period and the LGLs do not proliferate under these conditions, the actual E:T ratios at the time of NK cell activation are probably much lower than stated here.)

The induced natural killer cell mediated cytotoxicity (NK-CMC) is not associated with any direct cytotoxic or growth inhibitory effects of the extracts on the S180 cells. The SIE-induced NK-CMC can be demonstrated over a tremendous dose range, with no observable effect on S180 cell growth or viability in the absence of NK cells (0_NK effects). Microscopic observations of NK/S180 co-cultures treated with SIE have described a clustering and clumping of NK cells in “activated” cultures. The clustering of NK cells appears to be especially focused around S180 cells. Time course experiments indicate that 2-3 days of exposure of NK cells to the SIEs are required for the cytolytic activity to be induced. The induction of SIE-induced NK-CMC is blocked by treatment of the LGLs/S180 co-cultures with anti-asialo-GMI antibody, which specifically blocks NK cell cytolytic effects. These results indicate that NK cells are primarily involved in mediating the in vitro cytotoxic responses of SIEs against S180 tumor cells.

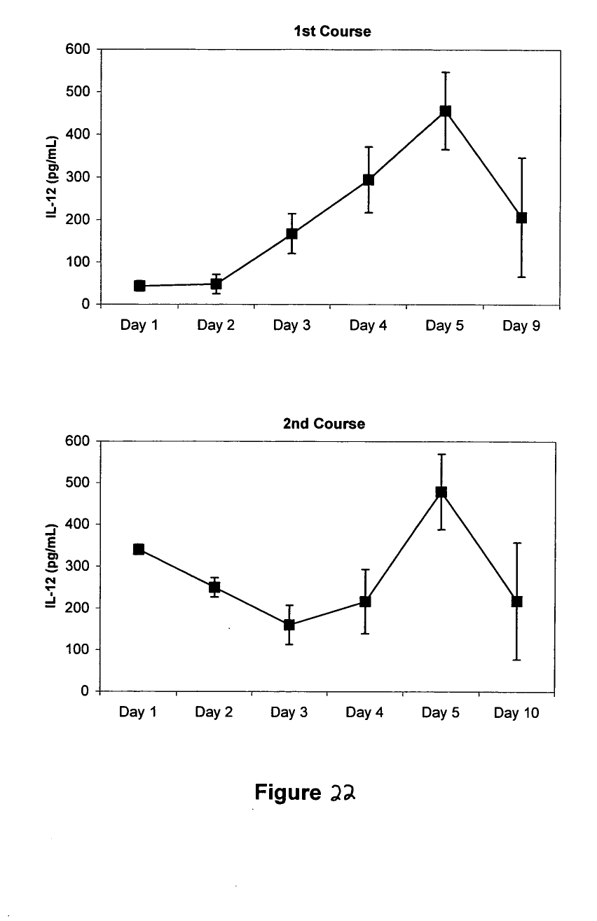

The induction of NK-CMC by SIEs in vitro is closely associated with an increase in IFNγ levels in the media of these cultures. When the concentration of IFNγ ([IFNγ]) is measured in the conditioned media of LGL cultures exposed to SIEs, the [IFNγ] is dramatically increased within 18 hours of treatment. Dose response relationships show a closely linked association between increases in [IFNγ] and induced NK-CMC (FIG. 22). Treatment of NK/S180 cell co-cultures with a neutralizing antibody against mouse IFNγ (anti-mouse IFNγ antibody), markedly reduces and/or blocks NK-CMC induced by SIEs. However, adding mouse recombinant IFNγ directly to NK/S180 co-cultures only results in slight increase in NK-CMC, far below the maximal NK-CMC induced by SIEs. These results suggest that increased [IFNγ] induced by SIEs is an early and required event in mediating SIE-induced NK-CMC but is not by itself sufficient to induce NK-CMC.

The rapid induction of IFNγ in LGL cultures by SIEs provided a more rapid method to test for activity in various purification stages. An IFN assay was devised and was used for several years to provide a one-day test of activity.

A large number of biological response modifiers have been reported to influence NK-CMC. We have screened a number of these factors in our NK-CMC assay in an attempt to duplicate SIE-induced activity in vitro. Table 16 lists the agents that have been tested.

TABLE 16
Summary of Effects of Various
Cytokines¹ on NK-CMC and DC Assays.

| | NK-CMC | DC/mIL12 |
|---|---|---|
| Cytokines | Activity | Release² |
| Interleukins | | |
| IL-1β | −³ | − |
| IL-2 | +⁴ | − |
| IL-3 | − | − |
| IL-4 | − | + |
| IL-5 | − | − |
| IL-6 | − | − |
| IL-7 | − | nt |
| IL-10 | − | inhibitory |
| IL-11 | − | nt |
| IL-12 | ++⁵ | na |
| IL-15 | − | nt |
| IL-18 | ++ | − |
| Interferons | | |
| IFNα | − | nt |
| IFNγ | +⁶ | − |
| Defensins, Anti- | | |
| microbial peptides | | |
| Corticostatin | − | nt |
| Magainin I | − | nt |
| Magainin II | − | nt |
| Cecropin A | − | nt |
| Cecropin B | − | nt |
| Cecropin P1 | − | nt |
| Defensin 1 | − | nt |
| Chemokines | | |
| MIP-1α | − | nt |
| JE/MCP-1 | − | nt |
| KC | − | nt |
| Misc. Cytokines, | | |
| Growth Factors | | |
| TNFα | +⁵ | − |
| TGFα | − | nt |
| TGFβ | − | − |
| FGFbasic | − | − |

¹Each agent was tested in the in vitro NK assay (LGL assay) over a wide range of concentrations both in the presence and absence of IL-2 (125 U/ml).
²Agents were tested for synergism and inhibition.
³Indicates little or no additive or synergistic effect, nt = not tested, na = not applicable.
⁴IL-2 significantly increases NK-CMC only at high concentrations.
⁵IL-12 synergistically increases NK-CMC at low concentrations of IL-2. However, these effects, unlike MEX or BEX, are restricted by NK:S180 cell ratios (see text). Weakly activates NK cells in the presence of IL-2 (maximal response < 50% cytotoxicity).
⁶Weakly activates NK cells in the presence of IL-2 (maximal response < 50% cytotoxicity).

Only IL-12, IL-18, TNFα, and IFNγ have demonstrated any capability to increase NK-CMC when tested under the same conditions as BEX (enriched ARP). However, TNFα and IFNγ were relatively weak enhancers when compared to BEX. Furthermore, as described below, the capacity of IL-12 to increase NK-CMC is linked to its production by dendritic cells in the LGL mixture. Finally, IL18, while a major component of the A fraction (DEAE flow-through fraction), is not present in the C fraction (DEAE eluted fraction). Therefore, no known enhancer of NK-CMC tested under the in vitro conditions of our LGL/S180 assay system stimulates NK cell mediated cytotoxicity in a fashion similar to extracts from the small intestine.

Dendritic Cell Activation

We have recently established that dendritic cells (DCs) play a central role in activities induced by ARP in vitro. This has been based on the observations that removal of DCs from the standard NK assay eliminates overall NK-CMC activity. Furthermore, BBX-01c induces mIL-12 (murine IL-12) release from enriched DC suspensions obtained with magnetic assisted cell separation (MACS). Anti-mIL-12 (neutralizing antibody) also markedly reduces BBX-induced NK-cell-mediated-cytotoxicity (NK-CMC).

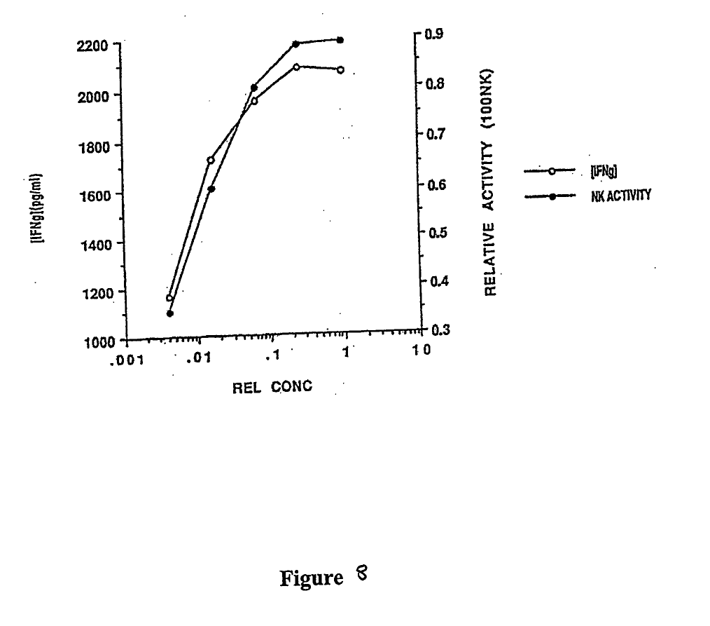

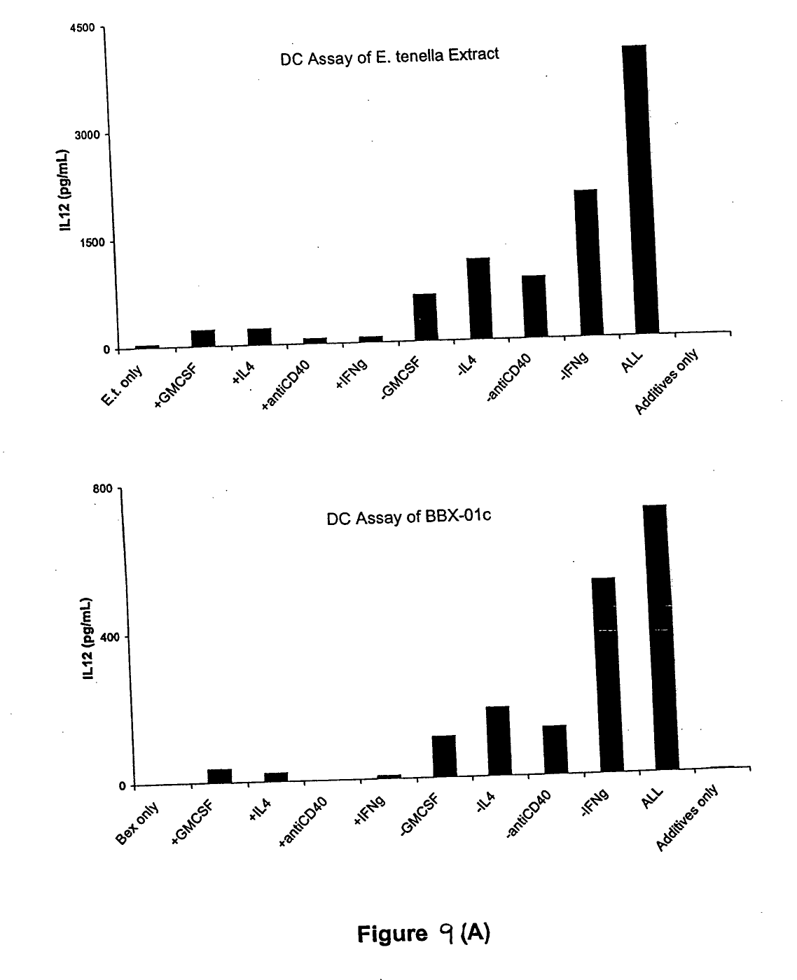

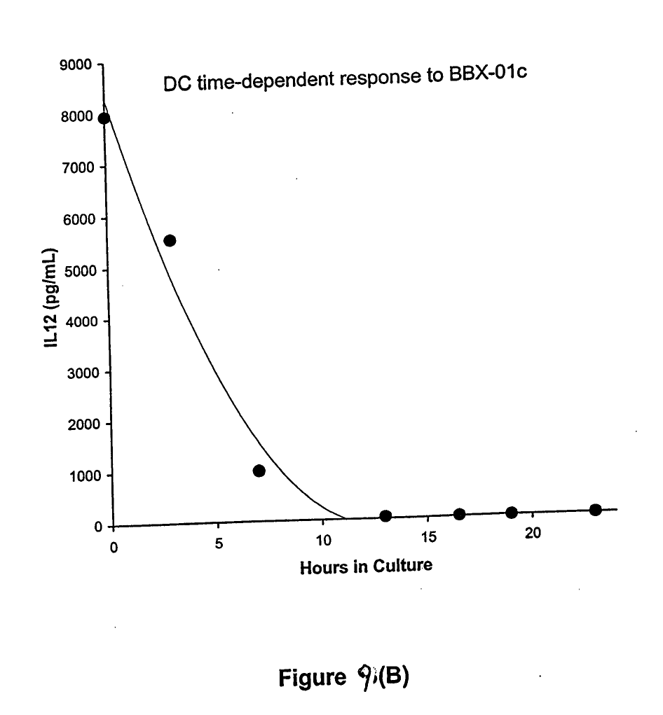

Attempts to exploit these observations into a useful assay for BBX-01c material were largely limited because BBX-01c induced only a modest amount of mIL-12 from spleen DCs in the absence of other activating factors. Exploration of other dendritic cell agonists and co-stimulatory molecules revealed powerful synergisms with the C component of BBX (FIG. 9(A)—lower panel). The four additives give maximal response in the presence of BBX-01c, but have no activity in its absence. Removal of one of the four additives results in varying reductions in IL-12 release. The anti-CD40 antibody, which substitutes for CD40 ligand, is the most important synergistic agent, followed by GM-CSF and then IL-4. The latter two cytokines are known to lead to maturation of dendritic cells, while CD40 ligand mimics the presence of CD4 T-cells and causes dendritic cells to release their activating cytokines. The E. tenella extract behaves in a virtually identical manner to the BBX sample (FIG. 9(A)—upper panel), which reinforces the similarity of the two active molecules, but more importantly, indicates that no other molecule of the small intestine tissue is part of the activity.

Optimization of the four additives has created a very robust assay for BBX-01 c and Eimeria activity. The DC/mL-12 release activity correlates extremely well with NK-CMC activity in samples from Superdex, HIC, various HPLC chromatographic separations as well as other fractionations. Furthermore, dose/response relationships indicate that the DC/mL-12 release assay is significantly more sensitive than the NK-CMC assay in detecting low levels of the active C component in BBX-01c. Other cytokines that were reported to be active in dendritic cells were also tested for synergism with C, these are reported in Table 16, supra.

Another important observation regarding the activity of BBX is the need to use freshly isolated spleen cells from which to purify dendritic cells. If DC-like cells are created in culture from peripheral blood monocytes by incubation with IL4 and GM-CSF, they are not responsive to BBX. Furthermore, if freshly isolated spleen cells are allowed to acclimate to culture conditions, they loose their responsiveness. This is shown in FIG. 9(B). It appears that a particular stage of dendritic cell maturation is required for activity. It appears that a particular stage of dendritic cell maturation is required for activity. The current immunotherapy protocols that expose dendritic cells to tumor antigens in vitro may not benefit from Eimeria antigen stimulation. Whereas those protocols that operate in vivo may be greatly enhanced.

### 13.5 Induction of Interleukin-12

One of the major effects of an ARP both in vitro and in vivo is the induction of interleukin-12 (IL12) release from dendritic cells. IL12 has been proposed for a variety of uses, e.g., in immune regulation. However, such uses have been limited by severe toxicity associated with administration of IL12. An ARP of the invention provides an alternative to systemic IL12 administration and provide the benefits of IL12 administration without the associated toxicity.

The preliminary assessment of the pharmacodynamics of an ARP molecule in vivo was tested in mice utilizing the extremely high sensitivity of the DC/mIL12 bioassay to detect ARP at very low concentrations. Mice were injected either ip or sc with a single dose of ARP EtU3 (352.5 ng active protein) diluted in 0.5 ml 0.1% BSA in PBS or a single dose of E. tenella extract (containing 36 ng of active ARP). Mice were bled via orbital sinus puncture technique using light ether anesthesia at the following time intervals: 1 hr, 3.5 hrs, 7.5 hrs, 12 hrs, 24 hrs, and 42 hrs. Blood was collected into heparinized micro-hematocrit capillary tubes and centrifuged for 10 min at 7000 rpm. Blood plasma was isolated and stored at +4° C. until the blood collection was completed.

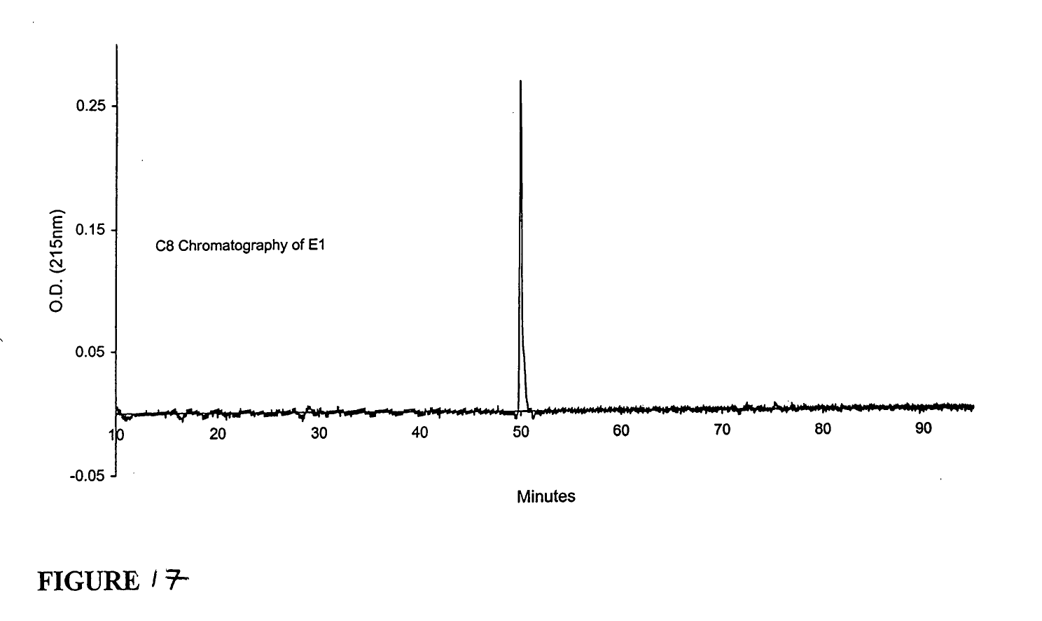

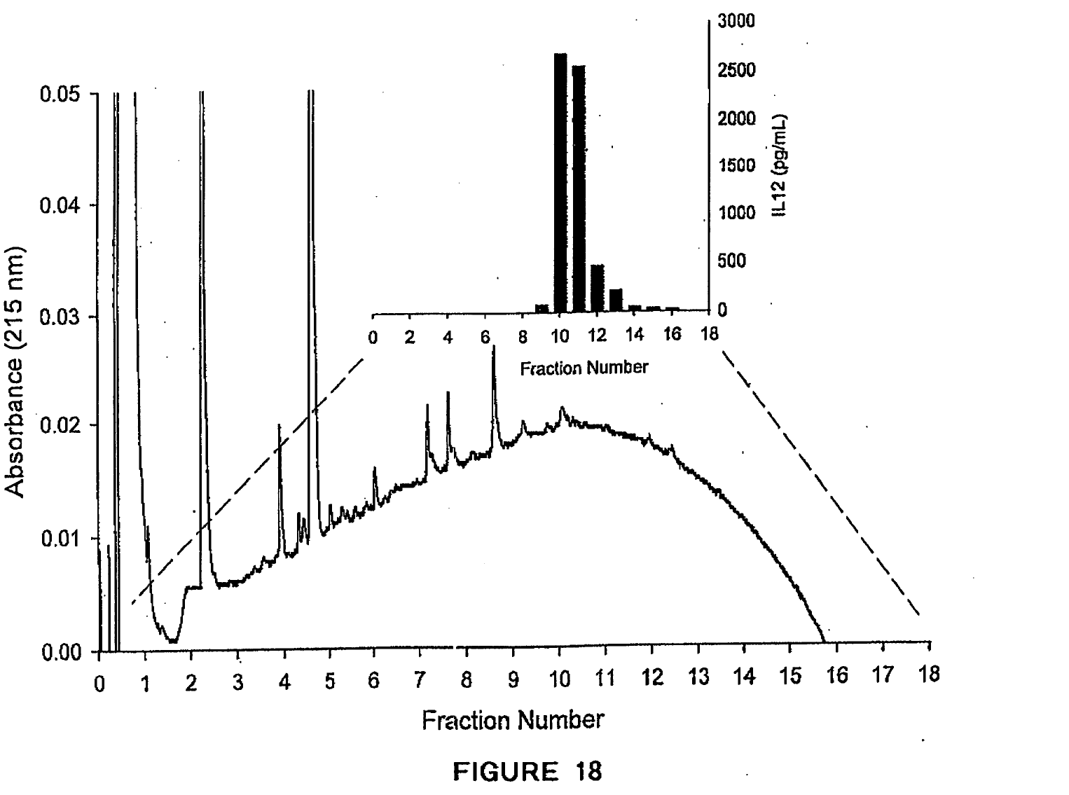

To assess the degree to which ARP can induce IL12 within the animal, aliquots of the plasma samples were diluted into the culture media and directly applied to the IL12 ELISA. The results, from three mice injected ip and one mouse injected sc for each treatment, are shown in FIG. 18. Plasma IL12 reached a peak at approximately 5 hours post injection and exhibited an exponential rise in concentration, with a rate constant 0.92 hr-1, and an exponential decay in concentration, with a rate constant of −0.15 hr-1.

These experiments have demonstrated proof of principle for the induction of IL12 from E. tenella-derived ARP drug treatment. The level of ARP injected in this experiment is significantly above the effective dose range in mice (˜0.05-5 ng/mouse/day), thus the rise and fall rates for induced in vivo IL12 must be considered tentative.

### 14. EXAMPLE 9

### Other Assays Used in the Purification of ARP LAL for Endotoxin

Endotoxin is routinely examined by the LAL gel clot assay (obtained from the Associates of Cape Cod Incorporated, 704 Main St., Falmouth Mass.). Samples were taken at various points during the purification process (Stage 1 and 2 processing) and were tested for endotoxin levels using the standard method described by the manufacturer. Early stages of intestine processing contained endotoxin above acceptable levels for human use, but these were reduced to safe levels by further purification. Processing on the Superdex™ 75 column provides a >10-fold reduction in endotoxin levels after each run. Therefore, re-processing the material through the column allowed reduction of endotoxin to any desired level, up to the native background levels in USP water. Target endotoxin levels have been set to yield material with a final concentration that meets the FDA guideline for parenteral drugs (<5 EU/kg). For the highest proposed dose of BBX-01 (100 mg/person), this yields a maximal target endotoxin level of approximately 70 kg/person×5 EU/kg÷100 mg/person, or 3.5 EU/mg for a 70 kg patient. Verification of the pyrogen safety of BBX-01 has been provided by independent testing at STS, Rush, N.Y. The rabbit pyrogen test on Superdex™ re-processed material, which had an endotoxin level of 0.4 (+0.2) EU/mg, yielded no pyretic effect.

Renaturation

Renaturation of collected fractions containing ARP, after reverse phase chromatography, SDS-gel electrophoresis (e.g., determining molecular weight), iso-electrofocusing (determining of pI), and some chemical modifications, used the method described for a different application (refolding of insoluble recombinant proteins) by Cabilly et al. Proc Natl Acad Sci USA. 81(11):3273-7 (1984), with several modifications. The fractions, collected from C-4, C-8, or C18 HPLC column chromatography, or extracted from slices of acrylamide gel, were put into a molecular porous dialysis tubing with MW cut-off of 6-8,000 kD (Spectra/Por, Houston, Tex.). BSA was added to each bag to a final concentration of 0.5 mg/mL. The bags were then dialyzed at 4° C. for 16 hr against 50 volumes of 8M urea/50 mM sodium glycinate, pH 10.8, 1 mM cysteine, and 0.1 mM cystine, this was followed by 24 hr dialysis against 100 vol of the same buffer containing 1 M urea, and finally, two-changes (8 hr, and 16 hr) against 100 vol of PBS with 2 mM EDTA, 1 mM cysteine, and 0.1 mM cystine. The fractions were filter sterilized by using Spin-X™ tube (Costar, Cambridge, Mass.) and diluted for in vitro assays.

SDS-PAGE Electrophoresis:

The Modular-Mini PROTEAN II™ Electrophoresis System (BioRad Cat# 165-2940) was used with 15% T, 0.8% C Tris/Glycine SDS Polyacrylamide gels. SDS-gel electrophoresis was performed according to Laemmli, U.K. (1970) Nature 227, 680-685. Gels were silver stained, photographed, and dried for storage (Gel Drying Kit from Promega, Fisher Cat# PR-V7120) or stored wet in sealed bags. For estimation of the molecular weight of ARP, gels were cut into slices, placed in a tube and extracted with buffer containing 0.1-0.5% SDS, followed by re-naturing. Extracts were tested for NK- and DC activity. SDS-PAGE standards (Bio-Rad) were used for calculation of molecular weight.

Chemical Modification Protocols

Chemical modifications of impure active bovine samples were used to determine the importance of various amino acids in an ARP. Reactions were performed according to “The Protein Protocols CD-ROM” (Walker, 1998, Protein Protocols on CD, Humana Press, Totowa, N.J.) designed for pure proteins. Modifications were added to allow the use of crude protein mixtures. For free COOH residue modification (in Asp, Glu, and C-terminus), the reagent EDC (1-ethyl-3-(3-dimethylaminopropyl)carbodiimide) was used. For His modification (ethoxyformylation), the reagent DEP (diethyl pyrocarbonate) was used. For Trp modification, Koshland's reagent (2-hydroxy-5-nitrobenzyl bromide) was used. For modification of guanidine groups (in Arg), the CHD (1,2-cyclohexanedione) reagent was used. For modification of free SH-groups (in Cys), two methods were utilized; a) Ellman's reagent, DTNB (5-5′-Dithiobis(2-nitrobenzoic acid)) and, b) NEM (N-ethylmaleimide). For modification of free NH₂ groups (amidination) (in Lys, and possible N-terminus), the amidination reagent MAH (methyl acetimidate hydrochloride) was used. For general oxidation/nitration reactions, TNM (tetranitromethane) was used. Treatment with, either NaBH₄, Na₂S₂O₄, or Na₂SO₃ was used for conducting reduction reactions (red-ox potentials for these reagents are 1.24, 2.96, and 0.92V). Hydroxylamine at basic pH was used for cleavage of the molecule at Asn-Gly site.

### 15. EXAMPLE 10

### Toxicology in Animals and Humans

Early Toxicity Studies

No toxicity was observed during the standard (ip.) treatment with small intestine extract or any of its purified fractions. No overt changes were noticed in the health of the animals, in their activity levels, or growth weights.

Acute toxicity of porcine small intestine extract was tested in 3 mice which were given up to 1.0 ml in a single dose. To test for induction of hypersensitivity or anaphylaxis, repeat doses of 0.5 and 0.9 ml porcine extract were given 7 and 8 days later, respectively. These did not induce any observable changes.

In high subcutaneous doses of BBX-01c, a very mild phenotypic change was noticed in the mice, but only during the 5 days of injection. There was no pathological effects observed in tissue samples from various organs.

Treated animals routinely showed splenomegaly. This has been observed for other immune stimulators, such as IL-12. Since there were no pathological changes to the spleen, the enlargement is considered a beneficial and desirable outcome of treatment with the intestinal extract

Safe Passage of BBX-01 in Dogs

Under the recommendation of the FDA, an initial safe passage study was performed on the original BBX-01 formulation on two male and two female beagles. This was contracted to MPI Research, Mattawan, Mich. The purpose of the study was to inject the animals with the initial dose that would be used in humans, without conversion to the lower body mass of the dog. This was considered by the FDA to provide some assurance that the first doses in humans could be administered safely and provide a base level from which dosage escalation could proceed. The summary of the report indicated that there was no adverse reaction to the drug.

Toxicity in Patients, BBX-01 (1^{st} Leg of Study)

The only toxicity observed in this leg of the phase 1 clinical trial was a mild, reversible erythema at the site of injection. This was not raised and did not itch. It occurred to large or smaller degree in all patients, but was not considered dangerous. No other side effects were observed in blood chemistries, cell counts, or in careful physical exams.

Toxicity in Patient, BBX-01c (2^{nd} Leg of Study)

The higher purity formulation, BBX-01c, did not exhibit the erythema of the previous formulation even with higher equivalent doses in half of the patients. Very high doses were not pursued, due to a lack of drug, but the one strong response in the germ cell ovarian cancer patient indicated that the therapeutic index for this material was likely to be high.

Preliminary Toxicity of E. tenella Extracts in Mice

A preparation from the oocysts of E. tenella, using the method described in Section 8, was injected at a single high dose into two mice. The mice tolerated the injection with no observable behavioral changes and no signs of any acute reactions have occurred.

Preliminary Toxicity of E. tenella Recombinant ARP, EtU3

Preliminary toxicity testing was conducted on a recombinant APR (EtU3). Three male BALB/c mice were each injected (ip) daily for 4 consecutive days with 4.3 μg of EtU3 in 1 ml buffer containing 0.1% BSA. A single non-injected mouse was retained as a control. Body weights were measured during and following the injections. Twenty-four days following the 4th injection the mice were given a 5th injection. Approximately 1 hour after the 5th injection, blood was sampled from each mouse for measurements of serum IL12 levels and for preliminary pharmacokinetic analysis. In addition, the following organs were removed and fixed in 10% buffered formalin for future histopathological analysis: spleen, liver, lung, kidney, adrenal, heart, and brain. No obvious indications of toxicity were observed during or following the treatment with EtU3. These include no significant changes in body weight, no “scruffiness”, no lethargy and no diarrhea.

Toxicity of rBBX-01 in Mice

The accute of toxicity of rBBX-01 (i.e. E1 formulated with 3% human serum albumin (HAS)) was evaluated in mice using an intraperitoneal (i.p.) route of administration. Three male mice were injected i.p. daily for 4 consecutive days with 4.27 μg/kg/day of rBBX-01. On day 27 each mouse received a 5^{th} dose. One hour after the 5^{th} dose, all animals were killed and necropsied. Selected organs and tissues were processed for histopathology.

There were no gross signs of toxicity in any of the treated mice. The body weights of the three treated mice showed an expected slight increase during the treatment period. Organs (liver, spleen, kidney, adrenal gland, heart, lungs and brain) were sampled, fixed and examined histopatholigically. No toxic effect was observed in any of the tissues sampled.

In a larger study, the acute and chronic toxicity of rBBX-01 was evaluated in mice under an intraperitoneal (i.p.) route of administration. Twenty-five male mice were injected i.p. with a single administration of rBBX-01 as follows: 5 mice were injected with vehicle control (3% HSA in PBS); 10 mice were injected with 400 ng rBBX-01; 5 mice were injected with 20 ng rBBX-01; 5 mice were injected with 1 mg rBBX-01. On day 4 post-injection 3 mice from the 400 ng treatment group were killed and necropsied. The remainder of the mice were necropsied on either day 17 or day 19. Selected organs and tissues were processed for histopathology. Body weights were recorded on days 4, 8, 10 and 13 of the study. Wet spleen weights were measured at necropsy on day 17 or 19.

There were no gross signs of toxicity in any of the treated mice. Body weights were not significantly different in any treatment group. There was a slight increase in wet spleen weight in mice treated with 400 ng rBBX-01 when compared with vehicle control treated animals. Organs and tissues were sampled, fixed and examined histopathologically. No significant toxic effect was observed in any of the tissues sampled.

Toxicity of rBBX-01 in Rats

A study was conducted to evaluate the potential toxicity of r-BBX-01 following five days of once daily subcutaneous administration in rats. The study consisted of three groups of male and female CD®[CrI: CD® (SD)IGS BR] rats (10/sex/group). Two groups received the test article, r-BBX-01, at dose levels of 7.5 and 300 ng/animal, and one group served as the control and received the vehicle, 3% human serum albumin (HSA) in phosphate buffered saline (PBS). The dose volume for all groups was 0.3 mL/animal. The first five animals/sex/group were necropsied on Day 6, while the last five animals/sex/group were necropsied on Day 15.

Observations for mortality and clinical signs, and body weight measurements were recorded throughout the study. Blood samples were collected on Days 2, 6, and 15 for the evaluation of clinical pathology parameters. At the respective interim and terminal necropsies (Days 6 and 15), complete necropsy examinations were performed, organ weights were measured, and selected tissues were collected, preserved for possible future processing and evaluation.

No treatment-related effects on survival, clinical signs, body weights, hematology/clinical chemistry evaluations, and macroscopic evaluations were noted during the study. No treatment-related organ weight changes occurred in either sex at the interim necropsy or in females at the terminal necropsy. However, terminal necropsy males exhibited a dose-related increase in prostate weights at 7.5 and 300 ng/animal (34% and 90% heavier than controls, respectively). In a subsequent study, rats receiving the same treatment showed a non-significant increase in prostate weight and microscopic evaluation of the prostate for all animals that were necropsied on Day 15 (Groups 1, 3, and 4; respective dose levels of 0, 7.5, and 300 ng/animal) determined that there were no treatment-related effects in this organ.

Based on the conditions and findings of this study, once daily—subcutaneous administration of r-BBX-01 to rats for five consecutive days was well tolerated at dose levels up to 300 ng/animal.

### 16. EXAMPLE 11

### Natural Abundance and Distribution of ARP

### 16.1 Animal Distribution

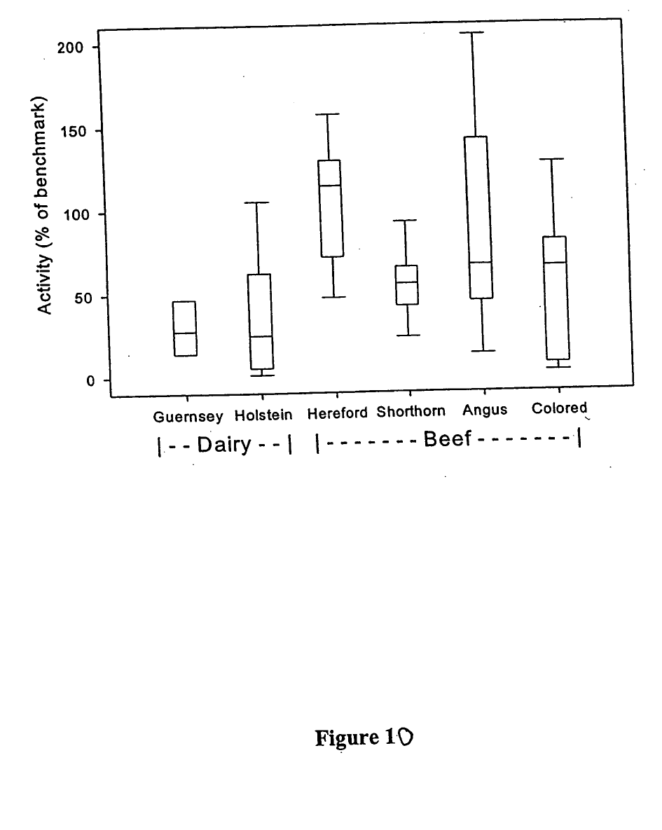

Cattle Breeds We obtained hundreds of intestinal segments from cattle and routinely observed the wide variation in activity between animals and pools of animal extracts. Some of this variation was attributed to different breeds, as shown in FIG. 10, but the intra-breed variation was also very high. With beef cattle routinely containing higher levels of activity, these were selected more often from the slaughterhouse. The higher yields allowed for more quickly reaching quantities sufficient for purification to homogeneity.

### 16.2 Tissue Distribution

Various Organs

A limited set of organs and tissues have been examined over the years to test the hypothesis that the observed activity is confined to the intestinal tract. Recent tissue samples from the spleen, liver, and lung of cattle have shown no activity in the DC and NK assays. However, due to the limited sample size and the sensentivity of the test, the absence of ARP in other organs is not conclusive.

Distribution of Activity Across the Intestinal Wall

70-90% of NK-activity, 80-100% of IFNγ-inducing activity and 80-100% of DC-activity could be extracted from the intestinal tissue with the use of 30 mM EDTA.

Small intestinal ileal sections were prepared and washed as described in Section 6. The washed pieces approximately 3 ft total length were cut into 2-3 cm pieces and incubated in PBS with 0.5-1 mM DTT 5-10 min to remove mucus. The solution was decanted and tissue pieces were stirred in 3-4×60 ml changes of PBS, 30 mM EDTA, 1 mM PMSF for 30-60 min at 40° C. The procedure removed cells from the mucosal layer containing most of the NK-, DC- and IFNγ-inducing activity as verified by analysis of the amount of activity in the residual tissue. Quantitative extraction of all activity types into the mucosal cell fraction is consistent with the known confinement of Eimeria infections to the intestinal mucosa.

### 17. EXAMPLE 12

### Domain Studies

The importance of the disulfide bridge has been verified in semi-pure samples of both bovine and E. tenella ARPs. Cleavage of bovine ARP at Asn-Gly site drastically reduced both NK-, and DC-activities. Since the bovine ARP has at least one such site in the middle of the molecule (FIG. 4), it shows that the resulting fragments of the ARP molecule contain no domains responsible for either NK-, or DC-activity.

A synthetic peptide (50-mer) was constructed from a combination of the variable region of the bovine molecule and the flanking regions of the 3-1 E protein (FIG. 4). This peptide was synthesized by Alpha Diagnostic International, Inc. and was received as a dry powder. It was tested in both the freshly reconstituted state and in a renatured form. Neither form exhibited any activity when tested over concentrations spanning 7 orders-of-magnitude starting at approximately 0.1 mg/mL. This synthetic peptide encompasses the domain between the disulfide bonds and its lack of activity indicates that either a different domain is important to dendritic cell binding or more of the molecule is needed for correct folding of this domain.

Experiments on recombinant ARP from E. tenella showed the that N-terminal end of the molecule can tolerate fusion of large non-related proteins and peptides without big changes in DC-activity. The truncated molecule with an ATG start codon in 6^{th} position (lacking GEADT) was at least as active as the full protein, but activity was lost when the start codon was moved to the 21st position (forming a truncated molecule lacking GEADTQAWDTSVREWLVDT). This suggests that the distal part of N-terminus is probably not involved in formation of any domains important for manifestation of at least DC-activity. Two C-terminal mutants of the E. tenella ARP (one with double exchange Tyr to His and Gly to Ser, the other one with exchange Gly to Ala) have been obtained and appeared to be equally active on DC-assay. Another mutant lacking three amino acids from C-terminus (SGF) retained significant activity while the mutants lacking 7, 10 or 14 amino acids from the C-terminus showed significantly lower (if any) DC-activity, it suggests that the C-terminal part of the molecule is part of some domain determining DC-activity. This part of the molecule contains no regions highly conserved in the known Apicomplexan ARPs. A common motif in the region is: T/S-A/I-L-A/N-F-A-E-(Y)-L, which, as judged by comparison of ESTs, is conserved within Eimeria, Neurospora, and the most often found ARP sequences from Toxoplasma.

### 18. EXAMPLE 13

### Antibodies to Variable and Constant Regions

Polyclonal antibodies to two 20-mer epitopes are being generated in rabbits by Alpha Diagnostics International, Inc. One epitope is from the non-conserved region of the bovine sequence and spans CRMFGASTDSGGDPYAELVQ. The other is from the E. acervulina sequence and spans CALYDEEKEQNKADALTTAL. In the first case, the cysteine is part of the molecule, while in the second case it was added at the beginning to allow conjugation to keyhole limpet haemocyanin, which is used to improve its antigenicity.

### 19. EXAMPLE 14

### Evidence for Hydrophobic Membrane Anchors

In some embodiments, ARP is an outer surface antigen of Eimeria parasites. We investigated the mechanism of its linkage to the cell membranes (GPI-linkage vs. integral membrane protein) by studying the possibility of ARP release by phospholipase C (PLC), the enzyme specific for GPI-linked proteins. The results presented in Example 4 suggest that ARP is GPI-linked to the outer membrane of E. tenella sporozoites.

Spontaneous release of ARP from the membranes was observed in these experiments, which is described in literature for other parasites as “shedding” of surface proteins (Zambrano-Villa et al., Trends Parasitol. 18(6):272-8 (2002)). The process was stimulated by elevated temperature, 10 mM EGTA, and was inhibited in the presence of 10 mM ZnCl₂, an inhibitor of endogenous protozoan PLC from Trypanosoma. These observations are consistent with the possibility of enzymatic nature of ARP release, probably due to endogenous PLC. However, non-enzymatic mechanisms are not excluded.

Many surface proteins of protozoa are anchored by GPI moieties (Ropert and Gazzinelli, Curr Opin Microbiol. 3(4):395-403 (2000); Almeida and Gazzinelli, J Leukoc Biol. 70(4):467-77 (2001)) and this moiety alone has previously demonstrated macrophage activation capacity (Magez et al., J. Immunol. 15; 160(4):1949-56 (1998)) and is known to bind to the toll-like receptor-2 (Campos, 2001, J. of Immunology 167:416-423), probably through its lipid moiety. Our experiments suggest that the release of ARP is regulated by protozoan metabolism through control of the GPI linkage and that the membrane linkage, disrupted by PLC is not required for activation of DC cells. Further evidence was generated by comparing the location of activity on C8 RP-HPLC separations of highly active E. tenella extracts with and without phospholipase treatments. In both cases, the activity remained unchanged and appeared in the same fractions. This distinguishes ARP from the DC activators that depend on lipid components, such as GPI itself, LPS, and bacterial lipoproteins (Brightbill et al., Science 285(5428):732-6 (1999); Aliprantis et al., Science 285(5428):736-9 (1999)).

### 20. EXAMPLE 15

### Identification of Proteins Homologous to the Eimeria 19 KD Sporozoite Antigen

Sequence homology searches were performed, using the E. acervulina ARP (SEQ ID NO:2) and the variable region of the Bovine Eimeria protein sequence of the invention in order to identify other proteins with similar sequence and/or structure to that found for the isolated peptide of the invention. The Basic Local Alignment Research Tool (BLAST)®, available through the website managed by the National Library or Medicine (www.ncbi.nlm.nih.gov/BLAST), was used to identify homologues of the bovine Eimeria protein (Table 17). Alternatively, databases containing Plasmodium falciparum genome sequences were used for comparisons between the Eimeria 19 kD sporozoite antigen and sequences of the Plasmodium falciparum genome, using the BLAST algorithm available at the following website: plasmodb.org/plasmodb/serylet/sv?page=blast (Table 17).

In brief, the Eimeria 19 kD sporozoite antigen peptide sequence was used in a BLAST query of 1) the database of Homo sapiens protein sequences with a total of 39,196 sequences, 2) the database of GenBank+EMBL+DDBJ+PDB (but no EST, STS, GSS, or phase 0, 1 or 2 HTGS) sequences with a total of 1,687,930, and 3) the database of GenBank+EMBL+DDBJ sequences from EST divisions with a total of 15,724,693 sequences. All of these databases were available for public searching through the website of the National Center for Biotechnology Information (NCBI) at the National Library of Medicine (NLM) and could be found at the aforementioned website.

Pairwise BLAST comparisons, within the database of Homo sapiens protein sequences were made using the “blastp” program option, which is suitable for protein-protein comparisons. No significant homology between the Eimeria 19 kD sporozoite antigen and the sequences on deposit in the Homo sapiens protein database were found.

Pairwise BLAST comparisons within the database of GenBank+EMBL+DDBJ+PDB (but no EST, STS, GSS, or phase 0, 1 or 2 HTGS) sequences were made using the “tblastn” program option, which compares the protein “Sequence 1” against the nucleotide “Sequence 2” which has been translated in all six reading frames. Significant sequence and structural homology was found between the Eimeria 19 kD sporozoite antigen and the known protein sequences on deposit in this database.

Pairwise BLAST comparisons within the database of GenBank+EMBL+DDBJ sequences from EST divisions were made using the “tblastn” program option, which compares the protein “Sequence 1” against the nucleotide “Sequence 2” which has been translated in all six reading frames. Significant homology was found between the Eimeria 19 kD sporozoite antigen and the known EST sequences on deposit in this database. Interestingly, only members of Apicomplexa resulted from this search.

The variable region sequence of the Bovine Eimeria protein of the invention was used in a BLAST query of the database of GenBank+EMBL+DDBJ+PDB (but no EST, STS, GSS, or phase 0, 1 or 2 HTGS sequences) sequences.

Pairwise BLAST comparisons within the database of GenBank+EMBL+DDBJ+PDB (but no EST, STS, GSS, or phase 0, 1 or 2 HTGS sequences) sequences were made using the “tblastn” program option, which compares the protein “Sequence 1” against the nucleotide “Sequence 2” which has been translated in all six reading frames. No reasonable homology was found between the variable region of the Bovine Eimeria protein of the invention and any known protein on deposit in the bovine database.

Identified ARPs have also been analyzed for conserved domains using Conserved Domain Database (CDD v. 1.60) and a Reverse Position-Specific BLAST® algorithm set to defaut parameters, which are available at the website ncbi.nlm.nih.gov/Structure/cdd/wrpsb.cgi. The analysis showed that the identified ARPs have a conserved PROF (profilin) domain, which is represented by profilin. Data showed that a preparation containing birch pollen (e.g., SEQ ID NO:13) was active in DC-assay. Based on the alignment on protein level, birch pollen profilin has 28% identity and 54% similarity to the E. tenella ARP; 31% identity and 57% similarity to the E. acervulina ARP.

TABLE 17
Representative BLAST ® Results

| | | | | | | Percentage |
|---|---|---|---|---|---|---|
| Input | | SEQ ID | | Bit | Percentage | of |
| Sequence | Accession # | NO. | Description | Score | of Identity | Positives¹ |
| EA 19 kD | 11561738 | 8 | Plasmodium (chr9.phat_327) | 116 | 39 | 56 |
| sporozoite | | | | | | |
| antigen | | | | | | |
| EA 19 kD | 11562004 | 9 | Plasmodium (chr9.gen_161) | 116 | 38 | 58 |
| sporozoite | | | | | | |
| antigen | | | | | | |
| EA 19 kD | 11562590 | 10 | Plasmodium (chr9.glm_372) | 100 | 40 | 58 |
| sporozoite | | | | | | |
| antigen | | | | | | |
| EA 19 kD | 11513601 | 11 | Latex profilin Hevb8 chain A from | 35 | 26 | 42 |
| sporozoite | | | Hevea Brasiliensis | | | |
| antigen | | | | | | |
| EA 19 kD | 2781152 | 12 | Profilin I from Arabidopsis Thaliana | 31 | | |
| sporozoite | | | | | | |
| antigen | | | | | | |
| EA 19 kD | 1942360 | 13 | Birch pollen profilin from Betula | 31 | | |
| sporozoite | | | Pendula | | | |
| antigen | | | | | | |
| EA 19 kD | 15224838 | 14 | Profilin I from Arabidopsis Thaliana | 31 | | |
| sporozoite | | | | | | |
| antigen | | | | | | |
| EA 19 kD | BG235691 | 21 | EtESTed11f08.yl Eimeria tenella S5- | 285 | 83 | 91 |
| sporozoite | | | 2 cDNA Neg Selected Eimeria tenella | | | |
| antigen | | | cDNA 5′ similar to TR: Q24777 | | | |
| | | | Q24777 19 KDA SPOROZOITE | | | |
| | | | ANTIGEN. [2] TR: Q03777;, MRNA | | | |
| | | | sequence | | | |
| EA 19 kD | CB070888 | 22 | TgESTzye83c06.yl TgME49 invivo | 169 | 51 | 72 |
| sporozoite | | | Bradyzoite cDNA size selected | | | |
| antigen | | | Toxoplasma gondii cDNA clone | | | |
| | | | TgESTzye83c06.yl 5′ similar to | | | |
| | | | TR: Q24777 Q24777 19 KDA | | | |
| | | | SPOROZOITE ANTIGEN. [2] | | | |
| | | | TR: Q03777;, MRNA sequence | | | |
| EA 19 kD | BM189297 | 23 | TgESTzyb37c04.yl TgRH Tachyzoite | 169 | 51 | 72 |
| sporozoite | | | Subtracted cDNA Library | | | |
| antigen | | | Toxoplasma gondii cDNA clone | | | |
| | | | TgESTzyb37c04.yl 5′ similar to | | | |
| | | | TR: Q24777 Q24777 19 KDA | | | |
| | | | SPOROZOITE ANTIGEN. [2] | | | |
| | | | TR: Q03777;, MRNA sequence | | | |
| EA 19 kD | AA167907 | 24 | CpEST.071 | 129 | 46 | 64 |
| sporozoite | | | uniZAPCpIOWAsporoLib1 | | | |
| antigen | | | Cryptosporidium parvum cDNA 5′ | | | |
| | | | similar to Eimeria acervulina 19 kDa | | | |
| | | | sporozoite antigen, MRNA sequence | | | |
| EA 19 kD | CA856473 | 25 | PfESToac08g05.yl Plasmodium | 122 | 38 | 57 |
| sporozoite | | | falciparum 3D7 gametocyte cDNA | | | |
| antigen | | | library Plasmodium falciparum 3D7 | | | |
| | | | cDNA 5′ similar to TR: Q24777 | | | |
| | | | Q24777 19 KDA SPOROZOITE | | | |
| | | | ANTIGEN. [2] TR: Q03777;, MRNA | | | |
| | | | sequence | | | |

¹Percentage of positives: the extent of similarity between two sequences, which can be based on percent sequence identity and/or conservation.

### 21. EXAMPLE 16

### Use of ARP as an Immunosuppressant by Initiation of Dendritic Cell Paralysis

While the primary activity of ARP is to induce IL-12 production in dendritic cells it has been noted that at high doses (˜1000 times stimulatory dose) ARP can suppress the production of IL-12. Based on this obeservation it maybe possible to use ARP as an immunosuppressant in the treatment of diseases that are caused by an overactive or inappropriate immune response. This hyposthesis was tested in the experimental autoimmune encephalomyelitis (EAE) model in SJL mice. EAE is induced in mice by immunization with spinal cord derived myelin basic protein which induces a disease in the mice which is very similar to multiple schlerosis (MS) in humans.

The SJL mice were immunized with the myelin basic protein on day 0 and on day 10 they received daily treatments of 0.1 μg, 1.0 μg or 10 μg ARP E1 given i.p. or with 5 mg Linomide given s.c. or with vehicle alone. Treatments were given daily for 5 days. Linomide is a drug that is known to inhibit EAE and is used as a positive control. EAE exhibits itself in mice as progressive paralysis beginning in the tail and progressing forward until death occurs. Severity of EAE is measured by means of the EAE score which starts at 0 for normal and increases to a score of 5 when death occurs. For ethical reasons mice are sacrificed when a score of 4 is reached. The experiment was terminated on day 15 and the mean EAE scores are shown in Table 18.

TABLE 18
Effect of ARP on EAE in SJL Mice

| Treatment | Day 15 EAE Score ± SD |
|---|---|
| Vehicle | 1.8 ± 1.9 |
| Linomide | 0.6 ± 1.3 |
| ARP E1 0.1 μg | 2.7 ± 1.3 |
| ARP E1 1.0 μg | 1.5 ± 1.6 |
| ARP E1 10.0 μg | 1.1 ± 1.5 |

Although the EAE scores in the ARP treated groups are not significantly different from the vehicle control, the trend in the scores is consistent with the proposed mechanism of action of ARP. At the lowest dose, where ARP is expected to act as an immunostimulant, it did appear to make the disease slightly worse. However, at the high dose where ARP's immunosupressant activity may expected the disease does appear to be supressed giving a score intermediate between the vehicle treated control and the Linomide positive control.

### 22. EXAMPLE 17

### Determination of the Number of CYS Residues in E1

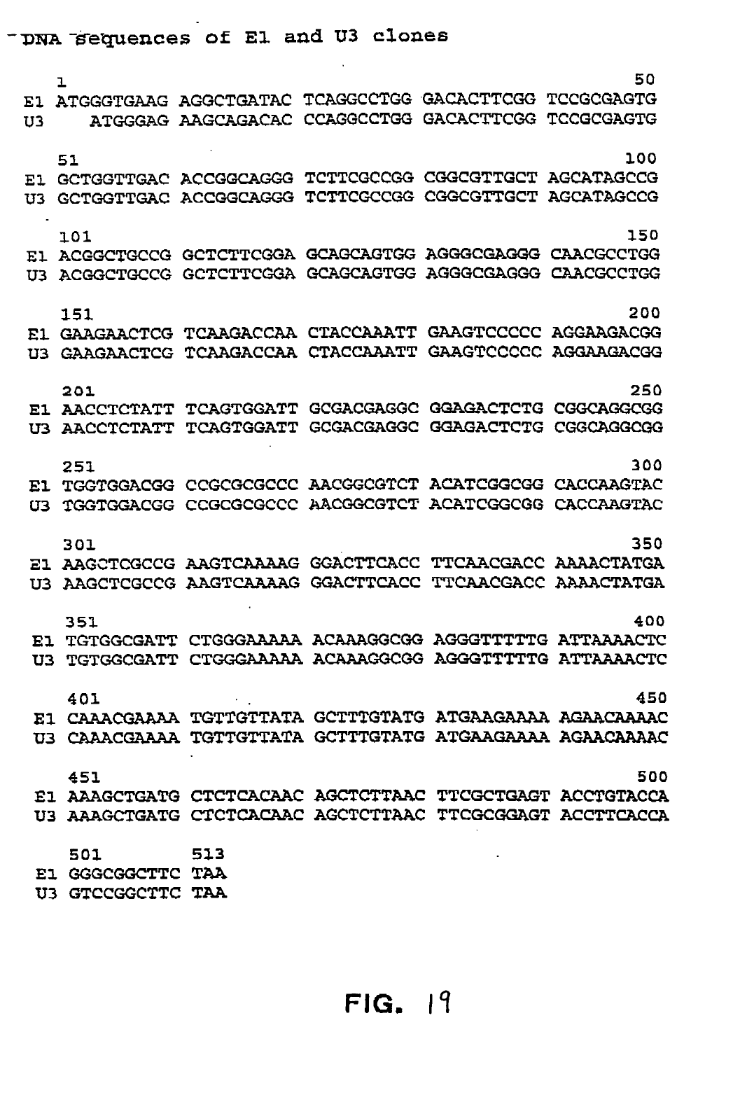

The amino acid sequence of E1 (ARP E1) as predicted by the nucleic acid sequence inserted into the production plasmid should contain two cysteines. Because of sulfur reactivity and the potential role of disulfide bond formation in protein stability, it is necessary to evaluate the sulfhydryl content of the recombinant protein as well as its redox state. The mass spectrometry data obtained on each batch yielded a consistent mass of 18,414 Da. This is the exact mass predicted for the amino acid sequence given for sequence E1 (FIG. 19) provided both sulfhydryls are reduced. A mass of 18,412 Da would be expected if a disulfide bond were present. While mass spectrometry is highly accurate, concluding that a mass of 18,414 Da is indicative of a reduced form is difficult in the absence of corroborating data. To obtain such data, the sulfhydryls of the protein were modified by Ellman's reagent and the mass re-determined.

Ellman's reagent is a dimer of two thio-nitro-benzoic acid residues linked through a disulfide bridge. This will react with the cysteine thiols in proteins to form mixed disulfides, yielding adducts of well defined mass. Depending on the local redox environment of the protein and the proximity of the thiols, these mixed disulfides can exchange, yielding a disulfide on the protein and the re-generation of Ellman's reagent. This reaction also yields a reduced Ellman monomer for each protein cysteine oxidized.

If the E1 protein contains two sulfhydryls, the first of two products from the Ellman's reaction should be the double adduct, with mass:

18,414.24+396.3 (Ellman)−2 (hydrogen lost)=18,808.54 Da. The second product should be the disulfide form of the protein with mass 18,412.24 Da.

The protocol for this reaction is given below, followed by the mass spectrometry data for the E1 drug substance. The only major components detected by mass spectrometry are the two expected products.

Results

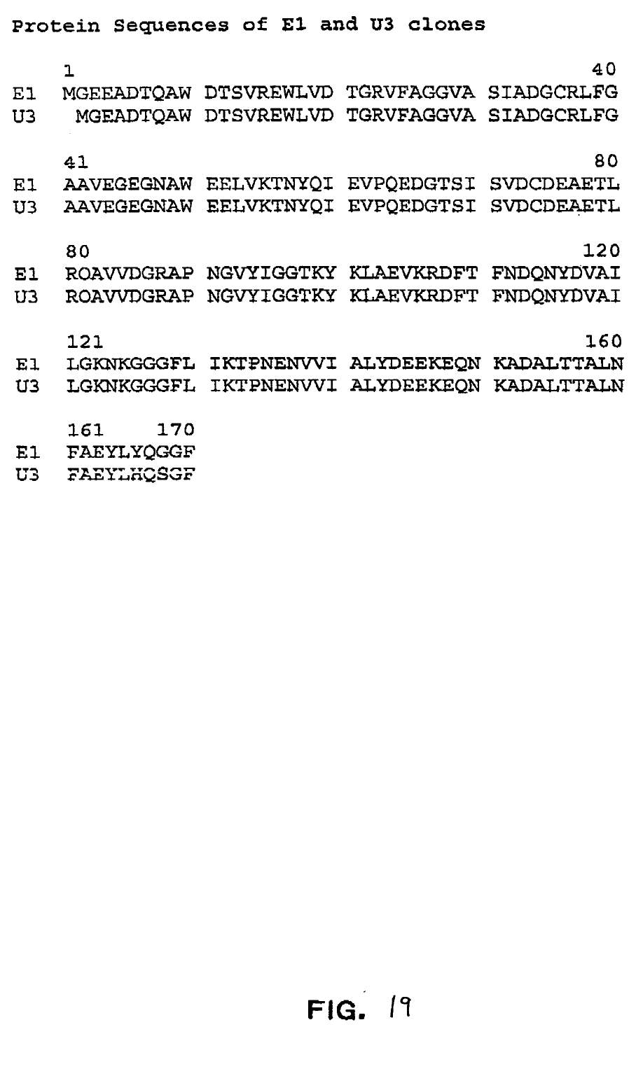

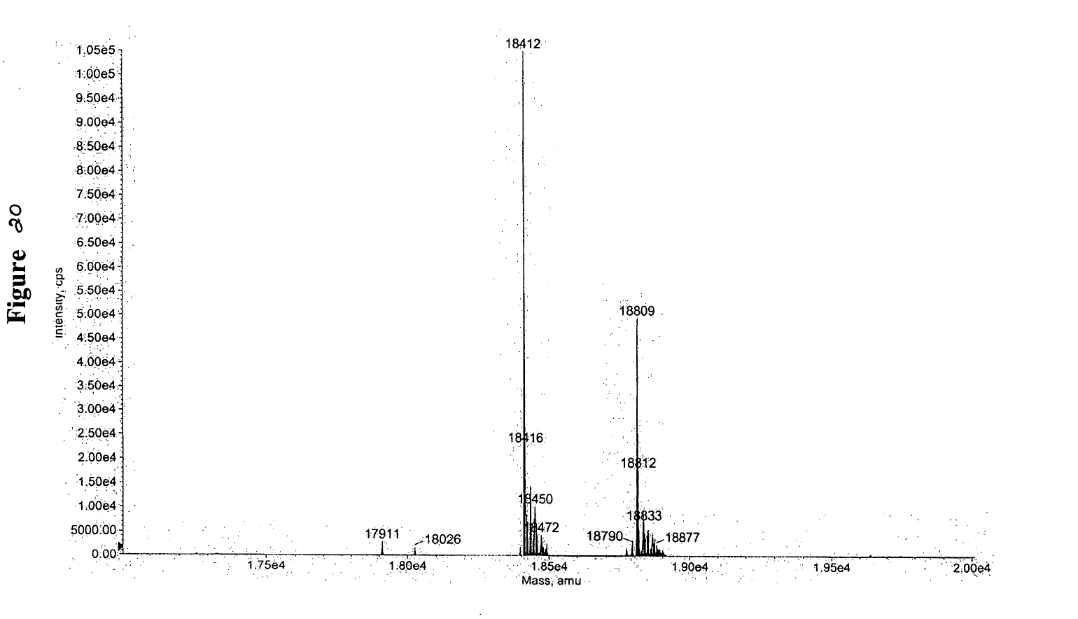

The deconvoluted Electrospray MS spectrum (FIG. 20) has the two masses expected for the reaction of Ellman's reagent with the E1 protein. No other masses of any significance are present.

The secondary result of this analysis is that the drug substance produced by each of the batches (molecular weight of 18,414 Da) is highly likely to exist in the reduced form.

Protocol

Chemicals:

1. DTNB (5,5′-Dithio-bis-(2-nitrobenzoic acid)=Ellman's reagent), Pierce, Cat.#22582
2. Cys (Cysteine), Sigma, Cat.# C6852
3. DMSO (Dimethyl sulfoxide), Sigma, Cat.# D8418
4. Gu-HCl (Guanidine chloride), Fisher, Cat.#BP 178-500
5. Tris (Trizma base), Sigma, Cat.#T1503
6. HCl (Hydrochloric acid), EMScience, Cat.#HX 0603-75
7. CH₃COO NH₄ (Ammonium acetate), Baker, Cat.#0596-01
8. H₂O, Baker, HPLC reagent, Cat.# 4218-03
   Stock Solutions:
1. Tris-HCl: 1M, pH 7.88
2. Ammonium acetate: 1M, pH 6.96
3. DTNB: 100 mM in DMSO
4. 10×PBS (used for Stage II)
5. Cys: 100 mM in PBS
   Working solutions:
1. Reaction buffer: 0.1M tris-HCl, pH 7.92-8.0
2. Dialysis buffer: 10 mM ammonium acetate pH 6.7
3. DTNB: 440 μM in 0.1M tris
4. Gu-HCl: 8M in 0.1M tris
5. Cys: 195.5 μM in PBS
6. PBS
7. Protein: E1-9, 1.8 mg/ml=195.5 μM in Cys
   Preparation of Working Solutions:

All working solutions except Gu-HCl and protein were prepared from stock solutions (# 1, 2, 3, 6) or by dissolving the solid reagent (#5) immediately before the experiment. Gu-HCl was prepared 1 week prior to the experiment and kept at room temperature. Protein was kept at −80° C. and was defrosted immediately before the experiment.

Immediately before the experiment all solutions were degassed in a dessicator on ice for 30 min, opened to air under nitrogen and brought to 22° C. (room temperature on that day) by 15 min incubation in 22° C. water bath for 15 min with occasional shaking.

Sample Treatment:

1. DTNB modification. 45 μl of test solutions (PBS, Cys, protein) were mixed with 225 μl of Gu-HCl and incubated at room temperature 15 min to denature the protein. 20 μl of DTNB solution was added on the plate reader and OD₄₁₄ was monitored for 1 hr with readings every 30 sec. The highest OD₄₁₄ obtained within 1 hr was used for calculating the concentration of the TNB²⁻ ion released with the use of ε₄₁₄=14,047 (Methods in Enzymology, 1983, 91, 49-60). PBS OD₄₁₄ reading was subtracted from Cys and protein readings. In the control protein sample DTNB was substituted by 0.1 M tris.

Results: Cys Sample: 73% DTNB-Modified.

Protein Sample: 34% DTNB-Modified.

2. Dialysis. After the reaction DTNB-modified and control protein samples were dialysed to remove Gu-HCl in separate beakers in 3×300 ml of 10 mM ammonium acetate for 3-10 hrs each.

3. Sample analysis. To determine the presence of modified protein both samples were analyzed by C8 RP-chromatography according to standard protocol. The number of modified Cys residues was estimated by determination of the molecular weight increase after DTNB modification (M-Scan, standard protocol). The minor component (probably, same as the minor component on C8 chromatogram (˜16% of total by peak height) showed 2 modified residues (Mol.wt. increase=397 Da). The major component was oxidized to 18412 Da.

### 23. EXAMPLE 18

### Disulfide Bond and ARP Activity

Directed point mutagenesis to change Cys residue into Ser residue was performed by using specific primers and PCR. Mutations (Cys35→Ser in one mutant and Cys73→Ser+Arg80→Trp in the other one) were confirmed by sequencing. Conditioned media and bacterial extracts were analyzed by DC-assay. Both modifications resulted in about 10-fold decrease of the activity compared to non-modified ARP. Since Arg80 is in a non-conserved region usually occupied by a hydrophobic amino acid, change it to hydrophobic Trp is likely of low importance. This implies that both Cys residues in the ARP molecule are important for DC activity, possibly due to formation of a disulfide bond.

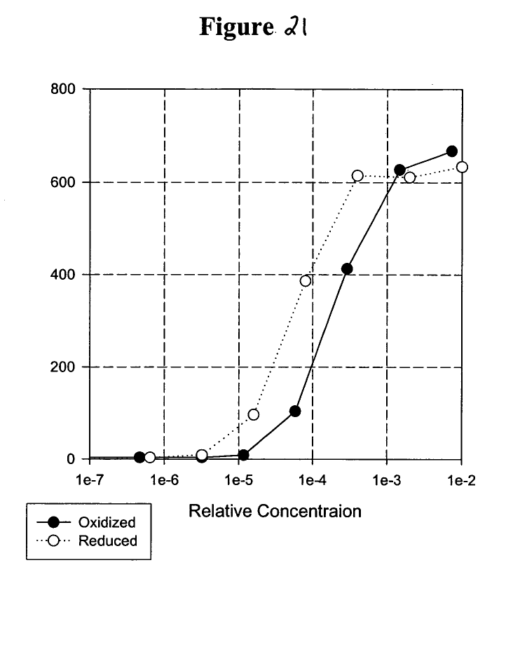

Both the oxidized and the reduced forms of ARP are active in the assay system. The reduced form is 2-5×more active that the oxidized form as shown in FIG. 21. However, since the DC assay is performed in conditions that allow the formation of a disulfide bond, it is not possible to say which form is directly interacting with DC cells.

### 22. REFERENCES CITED AND EQUIVALENTS

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method of treating or preventing an infectious disease in a subject wherein said infectious disease is caused by infection with a human immunodeficiency virus (HIV), comprising administering to the subject in whom such treatment or prevention is desired, a pharmaceutically effective amount of a pharmaceutical composition which provides a protein to said subject, wherein said protein has at least 25% identity to any one of the proteins selected from the group consisting of SEQ ID NO:1, SEQ ID NO:2, SEQ ID NO:28 or SEQ ID NO:30 as determined by a BLAST 2.0 algorithm set to default parameters, and wherein said protein possesses immunostimulatory activity.

2. The method according to claim 1, wherein said pharmaceutical composition provides said protein to a subject as a protein, a nucleic acid encoding said protein or a cell transfected with a nucleic acid encoding said protein.

3. The method according to claim 1, wherein said pharmaceutical composition provides said protein to a subject as:
(a) an isolated protein comprising SEQ ID NO:1;
(b) an isolated protein comprising SEQ ID NO:2;
(c) an isolated protein comprising SEQ ID NO:28;
(d) an isolated protein comprising SEQ ID NO:30;
(e) an isolated protein comprising SEQ ID NO: 3, 4, 5, 6 and 7;
(f) an isolated nucleic acid comprising a nucleotide sequence encoding any of (a)-(e), above; or
(f) a cell transformed with a nucleic acid comprising a nucleotide sequence encoding any of (a)-(e) above, said nucleotide sequence being operably linked to a promoter.

4. The method according to claim 3, wherein the pharmaceutical composition comprises an isolated protein comprising SEQ ID NO:1.

5. The method according to claim 3, wherein the pharmaceutical composition comprises an isolated protein comprising SEQ ID NO:2.

6. The method according to claim 3, wherein the pharmaceutical composition comprises an isolated protein comprising SEQ ID NO:28.

7. The method according to claim 3, wherein the pharmaceutical composition comprises an isolated protein comprising SEQ ID NO:30.

8. The method according to claim 3, wherein the pharmaceutical composition comprises an isolated protein comprising SEQ ID NO: 3, 4, 5, 6 and 7.

9. The method according to claim 1, wherein said pharmaceutical composition provides said protein to a subject as:
(a) an isolated protein that is the product of a process comprising the steps of:
(1) producing a cell extract of Eimeria infected tissue or cells;
(2) centrifuging the cell extract to produce a supernatant;
(3) fractionating the supernatant by ammonium sulfate to precipitate proteins to a pellet, and resuspending the pellet into a solution;
(4) applying the solution to a HIC column;
(5) eluting material bound to the HIC column to produce eluted material;
(6) dialyzing the eluted material to produce a sample for loading on a DEAE column;
(7) applying the sample to a DEAE column;
(8) eluting material bound to the DEAE column with buffer to produce an eluted fraction and concentrating the eluted fraction;
(9) applying the concentrated eluted fraction to a size exclusion column;
(10) collecting and pooling fractions eluted from the size exclusion column;
(11) applying the pooled fractions to a HIC column to remove calcium binding proteins;
(12) applying eluted fractions from the HIC column to a HPLC HIC column and collecting active fractions, wherein said active fractions have the ability to stimulate interleukin-12 release from dendritic cells, said dendritic cells having been isolated from a mammal within 10 hours of assaying said ability;
(13) dialyzing the active fractions; and
(14) applying the dialyzed active fractions to a HPLC DEAE column and collecting one or more active fractions, wherein said active fractions have the ability to stimulate interleukin-12 release from dendritic cells, said dendritic cells having been isolated from a mammal within 10 hours of assaying said ability;
(b) a recombinantly expressed protein;
(c) an isolated nucleic acid comprising a nucleotide sequence encoding (a) above; or
(d) a cell transformed with a nucleic acid comprising a nucleotide sequence encoding (a) above, said nucleotide sequence being operably linked to a promoter.

10. The method according to claim 9, wherein said isolated protein is the product of a process further comprising the steps of:
(15) applying the active fractions collected in step (14) to a reverse phase HPLC column; and
(16) eluting bound protein from the reverse phase HPLC column, to produce eluted protein, wherein said protein has the activity to stimulate interleukin-12 release from dendritic cells, said dendritic cells having been isolated from a mammal within 10 hours of assaying said activity.

11. The method according to claim 10, wherein said isolated protein is the product of a process further comprising a step of renaturing the eluted protein of step (16).

12. The method according to claim 9, wherein said cell extract is produced from Eimeria infected tissue, and wherein said tissue is bovine small intestine tissue.

13. The method according to claim 1 wherein said pharmaceutical composition is an isolated protein that is the product of a process comprising the steps of:
(a) producing a homogenate of a purified Apicomplexan organism or a cell infected with an Apicomplexan organism;
(b) centrifuging the homogenate to obtain a pellet;
(c) treating the pellet with a solution comprising a phospholipase;
(d) centrifuging the solution to obtain a supernatant;
(e) applying the supernatant to a reverse phase HPLC column; and
(f) eluting bound protein from the reverse phase HPLC column, to produce eluted protein, wherein said protein has the activity to stimulate interleukin-12 release from dendritic cells, said dendritic cells having been isolated from a mammal within 10 hours of assaying said activity.

14. The method according to claim 13, wherein said pharmaceutical composition is an isolated protein that is the product of a process further comprising a step of renaturing the eluted protein of step (f).

15. The method according to claim 9 wherein said pharmaceutical composition is an isolated protein that is expressed recombinantly.

16. The method according to claim 3, wherein said pharmaceutical composition provides said protein to a subject as a nucleic acid comprising a nucleotide sequence encoding the protein of any of (a)-(e).

17. The method according to claim 3, wherein said pharmaceutical composition provides said protein to a subject as a nucleic acid comprising a nucleotide sequence encoding the protein of any of (a)-(e), said nucleotide sequence being linked to a promoter operable in said subject.

18. The method according to claim 3, wherein said pharmaceutical composition provides said protein to a subject as a nucleic acid comprising a nucleotide sequence encoding the protein of any of (a)-(e), said nucleotide sequence being operably linked to a promoter.

19. The method according to claim 1, wherein said pharmaceutical composition provides said protein to a subject as:
(a) an isolated protein comprising SEQ ID NO: 3, 4, 6 or 7;
(b) an isolated protein comprising a variant of any one of SEQ ID NO:1, SEQ ID NO:2, SEQ ID NO:28 or SEQ ID NO:30, wherein said variant has only conservative amino acid substitutions relative to SEQ ID NO:1, SEQ ID NO:2, SEQ ID NO:28 or SEQ ID NO:30, respectively;
(c) an isolated protein comprising a variant of SEQ ID NO: 3, 4, 6 or 7, wherein said variant has only conservative amino acid substitutions relative to SEQ ID NO: 3, 4, 6 or 7, respectively;
(d) an isolated protein comprising a PROF (profilin) domain;
(e) an isolated Apicomplexan protein comprising an amino acid sequence encoded by a nucleic acid consisting of a nucleotide sequence hybridizable to SEQ ID NO:15, 16, 17, 18, 19, 20 or 27, or a complement of any of the foregoing SEQ ID NOs, under conditions of low stringency;
(f) an isolated Apicomplexan protein comprising an amino acid sequence encoded by a nucleic acid consisting of a nucleotide sequence hybridizable to SEQ ID NO:15, 16, 17, 18, 19, 20 or 27, or a complement of any of the foregoing SEQ ID NOs, under conditions of high stringency;
(g) an isolated nucleic acid comprising a nucleotide sequence encoding any of (a)-(f), above;
(h) a cell transformed with a nucleic acid comprising a nucleotide sequence encoding any of (a)-(f) above, said nucleotide sequence being operably linked to a promoter; and
wherein said pharmaceutical composition has the ability to stimulate interleukin-12 release from dendritic cells, said dendritic cells having been isolated from a mammal within 10 hours of assaying said ability.

20. The method according to claim 19, wherein said pharmaceutical composition provides said protein to a subject as an isolated protein comprising SEQ ID NO: 3, 4, 6 or 7.

21. The method according to any one of claims 8-13 or 18, wherein said pharmaceutical composition provides said protein to a subject as an isolated protein having a molecular weight of 18 to 25 kD as assayed by sodium dodecyl sulfate-polyacrylamide gel electrophoresis, and an isoelectric point (pI) between 4.0 and 4.7.

22. The method according to claim 19, wherein said pharmaceutical composition provides said protein to a subject as an isolated protein comprising a variant of any one of SEQ ID NO:1, SEQ ID NO:2, SEQ ID NO:28 or SEQ ID NO:30, wherein said variant has only conservative amino acid substitutions relative to SEQ ID NO:1, SEQ ID NO:2, SEQ ID NO:28 or SEQ ID NO:30, respectively.

23. The method according to claim 19, wherein said pharmaceutical composition provides said protein to a subject as an isolated protein comprising a variant of SEQ ID NO: 3, 4, 6 or 7, wherein said variant has only conservative amino acid substitutions relative to SEQ ID NO: 3, 4, 6 or 7, respectively.

24. The method according to claim 19, wherein said pharmaceutical composition provides said protein to a subject as an isolated protein comprising a PROF (profilin) domain.

25. The method according to claim 19, wherein said pharmaceutical composition provides said protein to a subject as an isolated Apicomplexan protein encoded by a nucleic acid consisting of a nucleotide sequence hybridizable to SEQ ID NO:15, 16, 17, 18, 19, 20 or 27, or a complement of any of the foregoing SEQ ID NOs, under conditions of low stringency.

26. The method according to claim 19, wherein said pharmaceutical composition provides said protein to a subject as an isolated Apicomplexan protein encoded by a nucleic acid consisting of a nucleotide sequence hybridizable to SEQ ID NO:15, 16, 17, 18, 19, 20 or 27, or a complement of any of the foregoing SEQ ID NOs, under conditions of high stringency.

27. The method according to claim 19, wherein the Eimeria nucleic acid comprises a nucleotide sequence encoding the protein of any of (a)-(f).

28. The method according to claim 19, wherein said pharmaceutical composition provides said protein to a subject as a nucleotide sequence encoding the protein of any of (a)-(f), said nucleotide sequence being linked to a promoter operable in said subject.

29. The method according to claim 19, wherein the transformed cell comprises a cell transformed with a nucleic acid comprising a nucleotide sequence encoding the protein of any of (a)-(f), said nucleotide sequence being operably linked to a promoter.

30. The method according to claim 1, wherein said pharmaceutical composition provides said protein to a subject as:
(a) an isolated protein comprising SEQ ID NO:1;
(b) an isolated protein comprising SEQ ID NO:2;
(c) an isolated protein comprising SEQ ID NO:28;
(d) an isolated protein comprising SEQ ID NO:30;
(e) an isolated protein comprising SEQ ID NO: 3, 4, 5, 6 and 7;
(f) an isolated nucleic acid comprising a nucleotide sequence encoding any of (a)-(e), above; or
(g) a cell transformed with a nucleic acid comprising a nucleotide sequence encoding any of (a)-(e) above, said nucleotide sequence being operably linked to a promoter.

31. The method according to claim 30, wherein the pharmaceutical composition comprises an isolated protein comprising SEQ ID NO:1.

32. The method according to claim 30, wherein the pharmaceutical composition comprises an isolated protein comprising SEQ ID NO:2.

33. The method according to claim 30, wherein the pharmaceutical composition comprises an isolated protein comprising SEQ ID NO:28.

34. The method according to claim 30, wherein the pharmaceutical composition comprises an isolated protein comprising SEQ ID NO:30.

35. The method according to claim 30, wherein the pharmaceutical composition comprises an isolated protein comprising SEQ ID NO: 3, 4, 5, 6 and 7.

36. The method according to claim 30, wherein said pharmaceutical composition provides said protein to a subject as a nucleic acid comprising a nucleotide sequence encoding the protein of any of (a)-(e).

37. The method according to claim 30, wherein said pharmaceutical composition provides said protein to a subject as a nucleic acid comprising a nucleotide sequence encoding the protein of any of (a)-(e), said nucleotide sequence being linked to a promoter operable in said subject.

38. The method according to claim 30, wherein said pharmaceutical composition provides said protein to a subject as a nucleic acid comprising a nucleotide sequence encoding the protein of any of (a)-(e), said nucleotide sequence being operably linked to a promoter.

39. The method according to any one of claims 35 or 38, wherein said pharmaceutical composition provides said protein to a subject as an isolated protein having a molecular weight of 18 to 25 kD as assayed by sodium dodecyl sulfate-polyacrylamide gel electrophoresis, and an isoelectric point (pI) between 4.0 and 4.7.

40. The method according to claim 19, wherein said pharmaceutical composition provides said protein to a subject as a nucleotide sequence encoding the protein of any of (a)-(f).

41. The method according to claim 19, wherein the transformed cell comprises a cell transformed with a nucleic acid comprising a nucleotide sequence encoding the protein of any of (a)-(e), said nucleotide sequence being operably linked to a promoter.

42. A method of treating or preventing disease in a subject wherein said infectious disease is caused by infection with human immunodeficiency virus (HIV), comprising administering to the subject in whom such treatment or prevention is desired, a therapeutically or prophylactically effective amount of a purified Apicomplexan organism or a protein-containing membrane fraction of a purified Apicomplexan organism.

43. The method according to claim 42, which comprises administering a purified inactivated Apicomplexan organism.

44. The method according to claim 42, which comprises administering sporulated oocysts of an Apicomplexan organism, and wherein the subject is not a natural host of the Apicomplexan organism.

45. The method according to claim 44, wherein said Apicomplexan organism is an Eimeria species.

46. The method according to claim 42, wherein said Apicomplexan organism or protein-containing membrane fraction is administered orally.

47. The method according to any one of claims 3, 9, 13, 19, or 30, wherein said pharmaceutical composition is administered with a metal-chelator.

48. The method according to claim 3, 9, 13, 19 or 30, wherein the administration of said pharmaceutical composition stimulates an immune response.

49. The method according to claim 42, wherein administration of said therapeutically or prophylactically effective amount of a purified Apicomplexan organism or a protein-containing membrane fraction of a purified Apicomplexan organism stimulates an immune response.

50. The method according to claim 3, 9, 13, 19, or 30, wherein the administration of said pharmaceutical composition stimulates an elevation of a systemic level of interleukin-12 (IL-12) in the subject.

51. The method according to claim 42, wherein the administration of said therapeutically or prophylactically effective amount of a purified Apicomplexan organism or a protein-containing membrane fraction of a purified Apicomplexan organism stimulates an elevation of a systemic level of interleukin-12 (IL-12) in the subject.

52. The method according to claim 3, 9, 13, 19, or 30, wherein said pharmaceutical composition comprises a protein that is encoded by the genome of an organism.

53. The method according to claim 52, wherein the organism is an Apicomplexan organism.

54. The method according to claim 53, wherein the Apicomplexan organism is an Eimeria species.

55. The method according to claim 3, 9, 13, 19, or 30, wherein said pharmaceutical composition comprises a molecule that is purified.

56. The method according to claim 3, 9, 13, 19, or 30, wherein said pharmaceutical composition comprises said isolated protein, said isolated protein comprising a disulfide bond.

57. The method according to claim 56, wherein the disulfide bond is reduced.

58. The method according to claim 56, wherein the disulfide bond is oxidized.

59. The method according to claim 3, 9, 13, 19, or 30, wherein said pharmaceutical composition comprises a glycosylated protein.

60. The method according to claim 3, 9, 13, 19, or 30, wherein said pharmaceutical composition comprises an unglycosylated protein.

61. The method according to claim 3, 9, 13, 19, or 30, wherein said pharmaceutical composition comprises a glycosylphosphatidylinositol (GPI)-linked protein.

62. The method according to claim 3, 9, 13, 19, or 30, wherein said pharmaceutical composition comprises a protein that is not glycosylphosphatidylinositol (GPI)-linked.

63. The method according to claim 3, 9, 13, 19, or 30, wherein said pharmaceutical composition comprises a native protein.

64. The method according to claim 3, 9, 13, 19, or 30, wherein said pharmaceutical composition comprises a protein that has been recombinantly expressed.

65. The method according to claim 3, 9, 13, 19, or 30, wherein said treating or preventing is with said pharmaceutical composition comprising an Eimeria protein.

66. The method according to claim 3, 9, 13, 19, or 30, wherein the subject is a human.

67. The method according to claim 3, 9, 13, 19, or 30, wherein the subject is a non-human.
